(19) United States
(12) Patent Application Publication
(10) Pub. No.: US 2014/0086899 A1
(43) Pub. Date: Mar. 27, 2014

(54) DIAGNOSTIC AND PROGNOSTIC ASSAY FOR A CONDITION OR EVENT OF THE VASCULAR SYSTEM

(71) Applicants: David William Howells, North Balwyn (AU); William John Wilson, Hornsby Heights (AU)

(72) Inventors: David William Howells, North Balwyn (AU); William John Wilson, Hornsby Heights (AU)

(73) Assignee: COMMONWEALTH SCIENTIFIC AND INDUSTRIAL RESEARCH ORGANISATION, CAMPBELL (AU)

(21) Appl. No.: 13/981,390

(22) PCT Filed: Jan. 27, 2012
(86) PCT No.: PCT/AU2012/000071

§ 371 Date: Dec. 13, 2013

(30) Foreign Application Priority Data
Jan. 28, 2011 (AU) 2011900274

(51) Int. Cl. G01N 33/68

(52) U.S. Cl. CPC G01N 33/6893
USPC 424/94.64; 436/501; 435/7.4; 435/7.92; 435/6.12; 506/9

(57) ABSTRACT

The present disclosure relates generally to the field of diagnostic and prognostic assays for a condition or event of a vascular system such as the cerebrovascular system. An assay is taught herein for monitoring progression of a condition or event of a vascular system such as the cerebrovascular system as well as determining the state or stage of the condition or event. The assay of the present disclosure is also useful in the stratification of a subject based on the stage or development of the condition or event.

### FILING DATA

This application is associated with and claims priority from Australian Provisional Patent Application No. 2011900274, filed on 28 Jan. 2011, entitled “Diagnostic and prognostic assay”, the entire contents of which, are incorporated herein by reference.

### FIELD

The present disclosure relates generally to the field of diagnostic and prognostic assays for a condition or event of a vascular system such as the cerebrovascular system. An assay is taught herein for monitoring progression of a condition or event of a vascular system such as the cerebrovascular system as well as determining the state or stage of the condition or event. The assay of the present disclosure is also useful in the stratification of a subject based on the stage or development of the condition or event.

### BACKGROUND

Bibliographic details of the publications referred to by author in this specification are collected alphabetically at the end of the description.

Reference to any prior art in this specification is not, and should not be taken as, an acknowledgment or any form of suggestion that this prior art forms part of the common general knowledge in any country.

Adverse conditions and events of vascular systems represent a major course of morbidity and mortality in many populations. This is particularly the case for the cerebrovascular system. One such condition of a vascular system such as the cerebrovascular system is stroke which is the third most common cause of death amongst populations within most Western countries (Strong et al. (2007) Lancet Neurol 6:182-187,). It also represents a significant cause of disability ranging from minor to extremely disabling. Approximately 85% of strokes are of ischemic origin due to vessel occlusion and approximately 15% of strokes are due to vessel rupture and intracranial hemorrhage (ICH). To put the significance of stroke into perspective, the World Heath Organisation estimates that globally 20 million people suffer a stroke each year of which approximately 5.1 million die. In addition, over 200 million people suffer “silent strokes” which nevertheless contribute to some form of vascular dementia.

Approximately 20% of patients die within the first month of having a stroke with 37% dying in the first year. Approximately 24% to 50% of survivors exhibit some form of dementia or cognitive decline within a year of the stroke, requiring a certain amount of dependency on others.

Quite apart from the personal effects on the quality of life on the survivors or on members of their families, the high incidence of stroke places an enormous burden on already struggling private and public health care systems.

The treatment of stroke is generally divided into four categories: intravenous thrombolysis, oral aspirin, management in a dedicated stroke unit (i.e. patient care) and hemicraniectomy, when malignant brain oedema requires surgical decompression (Donnan et al., (2008) Lancet 371:1612-1523).

The most specific and efficacious of these treatments is thrombolysis with tissue plasminogen activator (tPA) if given within 4.5 hours of ischemic stroke onset (Hacke et al. (2008) N. Engl. J. Med 359:1317-1329). Results of tPA treatment range from a benefit of one patient per 3.6 patients treated within 90 minutes of stroke to one patient benefited per 19.3 patients 270-360 minutes after stroke. However, generally, only about 1% of stroke victims receive thrombolytic treatment due to the need to establish whether the stroke is of ischemic or hemorrhagic in nature, the absence of imaging resources in many hospitals or point of care facilities and the presence of high blood pressure which is contraindicated for patients receiving thrombolytic therapy (Hacke et al. (2004) Lancet 363:768-774; Lees et al. (2010) Lancet 375:1695-1703).

It is apparent that restoration of blood flow promptly after onset of ischemic stroke has the potential to assist a much larger group than currently are treated using the “clock” time period of from 0 to 4.5 hours from onset.

There is a need, therefore, to be able to stratify a patient to establish a time line from a biological and temporal perspective in the evolution of stroke and other thromboembolic and non-thromboembolic conditions and events.

### SUMMARY

Throughout this specification, unless the context requires otherwise, the word “comprise”, or variations such as “comprises” or “comprising”, will be understood to imply the inclusion of a stated element or integer or method step or group of elements or integers or method steps but not the exclusion of any other element or integer or method step or group of elements or integers or method steps.

As used in the subject specification, the singular forms “a”, “an” and “the” include plural aspects unless the context clearly dictates otherwise. Thus, for example, reference to “a biomarker” includes a single biomarker, as well as 2 or more biomarkers; reference to “an event” includes a single event or two or more events; reference to “the disclosure” includes single and multiple aspects taught by the disclosure. All aspects taught herein are encompassed by the term “invention”. All aspects of the invention are enabled within the width of the claims.

The use of numerical values in the various ranges specified in this specification, unless expressly indicated otherwise, are stated as approximations as though the minimum and maximum values within the stated ranges were both preceded by the word “about”. In this manner, slight variations above and below the stated ranges can be used to achieve substantially the same results as values within the ranges. Also, the disclosure of these ranges is intended as a continuous range including every value between the minimum and maximum values. In addition, the present disclosure extends to ratios of the levels of 2 or more biomarkers providing a numerical value associated with progression of a condition or event of a vascular system such as the cerebrovascular system.

A method is taught herein for monitoring progression of a condition or event of a vascular system such as the cerebrovascular system in a subject. The method comprises determining the levels or ratios of levels of 2 or more biomarkers and comparing these levels or ratios to a control. The control comprises pre-determined levels or ratios from subjects with a known status with respect to the condition or event. The biomarkers are selected on the basis that their levels dynamically alter during evolution of the condition or event in a relatively statistically reproducible manner within a pre-determined period. Hence, the biomarker is selected on the basis that its level increases or decreases over a time period. Consequently, a physiological time period is thereby established wherein the levels or ratios of levels of the biomarkers at least correlate to the levels or ratios of levels observed in a pre-determined “clock” time period following onset of the condition or event. The biomarkers are used to define a physiological time period following onset of the condition or event rather than clock time defining this period. It is proposed that the physiological time period is wider than the corresponding clock time period following onset of the acute condition or event. This has the consequence of re-defining various therapeutic windows. This has relevance to both acute conditions and events as well as chronic conditions and events.

The acute condition or event of a vascular system such as the cerebrovascular system may be ischemic or hemorrhagic in nature or origin. Examples of ischemic conditions and events include ischemic stroke and ischemic myocardial infarction. Examples of a hemorrhagic condition or event is hemorrhagic stroke. The biomarkers are also proposed be useful in distinguishing between conditions or events such as differentiating between ischemic stroke and hemorrhagic stroke. A chronic condition or event includes vascular dementia which may develop over time.

In an embodiment, the condition or event is ischemic stroke including its various manifestations such as pre-stroke, transient ischemic attack and acute ischemic stroke as well as other related thromboembolic events. Generally, such conditions and events have the potential to lead to some form of adverse neurological condition.

Useful biomarkers contemplated herein are those listed in Table 1 by Affymetrix gene identifier (ID) number. A particular set of biomarkers is provided in Table 2. The genes are listed by p-value of the F-statistic in decreasing order of significance across all time points. In relation to ischemic stroke, the levels or ratios of levels of 2 or more of the biomarkers correlate to levels within a pre-determined time period after onset. In an embodiment, the pre-determined time period is the therapeutic window for thrombolytic intervention, generally from about 0 hours to 4.5 hours or a sub-fraction thereof after onset. In accordance with the present disclosure, although on a “clock” basis, 4.5 hours may represent the cutoff point for thrombolytic treatment, the “physiological clock” is proposed herein to extend the 4.5 hours cutoff point to greater than 4.5 hours such as between 4.5 hours and 10 hours. Hence, more patients can receive thrombolytic treatment. In another embodiment, the condition may be chronic requiring ongoing treatment over months or years. In yet a further embodiment, the chronic condition may reach a point requiring medical intervention. It is proposed herein that the biomarker expression profile is used to define when a chronic condition or potential chronic condition enters a therapeutic window.

Of the biomarkers listed in Table 1, all biomarkers may be measured or from about 2 to about 20 biomarkers may be measured or from about 2 to about 15 such as 12 biomarkers may be measured. By “measured” means determining levels, concentrations or velocities of a biomarker. The value may be the actual level or a ratio of levels. The term “biomarker” extends to both a gene and a gene product such as a protein or mRNA transcript. In a most particular embodiment, the 2 or more biomarkers are selected from Table 2.

In an embodiment, the subject is a human. Hence, the assay of the present disclosure permits stratification of a subject on the basis of extent of progression of the condition or event based on physiological markers rather than on “clock time”. In an embodiment, the stratification enables identification of a therapeutic window for thrombolytic intervention. This window is defined as the levels or ratios of levels of biomarkers which equate to the levels or ratios at from about 0 hours to about 4.5 hours (or other pre-determined time period) after onset of the condition or event. It is proposed herein that the “physiological clock time” is longer than the 4.5 hour clock time recommended for thrombolytic treatment. For chronic conditions or events, the profile of biomarker expression may define when therapy is required. Onset of the condition may have been years earlier. Vascular dementia is one example.

A method is enabled herein for treating a subject who has or is suspected of having suffered a condition or event of a vascular system such as the cerebrovascular system. The method comprises determining levels or ratios of biomarkers which equate to the levels or ratios at a pre-determined time period. In the case of ischemic stroke, the pre-determined time period is 0 to 4.5 hours. This is the therapeutic window for thrombolytic intervention in the case of an ischemic condition or event. If the subject has biomarker levels or ratios instructive to this therapeutic window, then a thrombolytic agent is administered. In addition or alternatively, the treatment may involve oral aspirin administration, patient care and/or hemicraniectomy. In an example, the condition or event is ischemic stroke or ischemic myocardial infarction. The biomarkers include 2 or more selected from the list in Table 1 and such as 2 or more biomarkers selected from the list in Table 2. In another embodiment, the therapeutic window is defined when biomarker expression reaches a particular profile. This may be a time or age-based period or it may depend on the severity of symptoms. Hence, treatment is initiated when the expression profile of the biomarkers equates to levels corresponding to a set of symptoms.

The present disclosure further teaches the use of 2 or more biomarkers, the levels of which dynamically alter during evolution of an acute condition or event of a vascular system such as the cerebrovascular system in a subject, in the manufacture of a diagnostic assay to determine the extent of progression of the condition or event. A biomarker which remains stable over a time period may also be selected as a control or to use in a ratio determination with a biomarker with increases or decreases over time. Furthermore, the present disclosure contemplates the use of a multivariate statistical model based on determining the levels or ratios of levels of 2 or more biomarkers to establish an expression profile of the biomarkers.

Kits and web-based assays are also enabled herein. A web-based assay is useful for point of care facilities for the rapid reporting of diagnostic information.

Tables 1 and 2 list biomarkers useful in the practice of the present assay by Affymetrix gene identifier (ID) number (Affymetrix gene identifier). Information on the genes can be obtained from the Affymetrix website [NetAffx (Trademark)] which can be accessed at http://www.affymetrix.com/analysis/index.affx. Where available, an ENSEMBL identifier is also provided which can be accessed through the ENSEMBL rat genome website at http://www.ensembl.org/Rattus_norvegicus. Where an ENSEMBL number is not available, the term “NA” is used. Although genes are listed in Tables 1 and 2, the term “biomarker” extends to both genes and gene products. A gene product may be a protein or mRNA transcript. The genes are listed by increasing p-value of the F-statistic.

TABLE 1
Biomarkers

| affymetrix gene ID | p-value for F-statistic | ENSEMBL annotations |
|---|---|---|
| 10722208 | 1.21E−26 | ENSRNOG00000014227 |
| 10810144 | 1.03E−24 | ENSRNOG00000023272 |
| 10940615 | 5.06E−24 | NA |
| 10736312 | 6.12E−24 | ENSRNOG00000011023 |
| 10826956 | 2.33E−23 | NA |
| 10763768 | 5.25E−23 | ENSRNOG00000004441 |
| 10748273 | 5.43E−23 | ENSRNOG00000011917 |
| 10922816 | 1.22E−22 | ENSRNOG00000014378 |
| 10783880 | 1.29E−22 | ENSRNOG00000020136 |
| 10707142 | 1.51E−22 | ENSRNOG00000013103 |
| 10826703 | 4.36E−22 | ENSRNOG00000010875 |
| 10909411 | 4.42E−22 | ENSRNOG00000006663 |
| 10828832 | 4.58E−22 | ENSRNOG00000001869 |
| 10705065 | 5.11E−22 | ENSRNOG00000020125 |
| 10721261 | 7.52E−22 | ENSRNOG00000037331 |
| 10864433 | 8.59E−22 | ENSRNOG00000010898 |
| 10732941 | 8.98E−22 | ENSRNOG00000028845 |
| 10728883 | 9.47E−22 | ENSRNOG00000020945 |
| 10893918 | 2.16E−21 | ENSRNOG00000013987 |
| 10892472 | 2.56E−21 | ENSRNOG00000037529 |
| 10823363 | 2.80E−21 | ENSRNOG00000029756 |
| 10826969 | 3.91E−21 | NA |
| 10769765 | 4.07E−21 | ENSRNOG00000003136 |
| 10735331 | 4.83E−21 | ENSRNOG00000004078 |
| 10824305 | 7.07E−21 | ENSRNOG00000019737 |
| 10922492 | 8.25E−21 | ENSRNOG00000015382 |
| 10826943 | 8.70E−21 | NA |
| 10810923 | 9.55E−21 | ENSRNOG00000014964 |
| 10836588 | 1.62E−20 | ENSRNOG00000006773 |
| 10880404 | 1.69E−20 | ENSRNOG00000013231 |
| 10812292 | 1.88E−20 | NA |
| 10849279 | 1.89E−20 | ENSRNOG00000018204 |
| 10933912 | 2.07E−20 | ENSRNOG00000038299 |
| 10751437 | 2.22E−20 | ENSRNOG00000038831 |
| 10766428 | 2.32E−20 | NA |
| 10730131 | 2.34E−20 | ENSRNOG00000013967 |
| 10703764 | 2.40E−20 | ENSRNOG00000038774 |
| 10775519 | 2.50E−20 | ENSRNOG00000002159 |
| 10926252 | 2.70E−20 | NA |
| 10808959 | 2.90E−20 | ENSRNOG00000010744 |
| 10894369 | 3.97E−20 | ENSRNOG00000039204 |
| 10924230 | 4.54E−20 | NA |
| 10725684 | 4.62E−20 | ENSRNOG00000018311 |
| 10792028 | 4.74E−20 | ENSRNOG00000011474 |
| 10868627 | 4.75E−20 | ENSRNOG00000014838 |
| 10749372 | 5.06E−20 | ENSRNOG00000002946 |
| 10808377 | 5.07E−20 | ENSRNOG00000016752 |
| 10832602 | 5.51E−20 | ENSRNOG00000001304 |
| 10927195 | 6.59E−20 | ENSRNOG00000011771 |
| 10800546 | 9.52E−20 | ENSRNOG00000015113 |
| 10831940 | 1.22E−19 | ENSRNOG00000022523 |
| 10828827 | 1.33E−19 | ENSRNOG00000000521 |
| 10751434 | 1.44E−19 | ENSRNOG00000028918 |
| 10933431 | 1.46E−19 | ENSRNOG00000003705 |
| 10747506 | 1.48E−19 | ENSRNOG00000019742 |
| 10862082 | 1.58E−19 | ENSRNOG00000010957 |
| 10716939 | 1.72E−19 | ENSRNOG00000011411 |
| 10817074 | 1.79E−19 | ENSRNOG00000021798 |
| 10738071 | 2.04E−19 | ENSRNOG00000009643 |
| 10703757 | 2.41E−19 | ENSRNOG00000038775 |
| 10755013 | 2.74E−19 | ENSRNOG00000001928 |
| 10703776 | 2.80E−19 | ENSRNOG00000014812 |
| 10740843 | 3.29E−19 | ENSRNOG00000029621 |
| 10865948 | 3.61E−19 | ENSRNOG00000007310 |
| 10762051 | 3.88E−19 | ENSRNOG00000001270 |
| 10775398 | 3.95E−19 | ENSRNOG00000002186 |
| 10759999 | 4.05E−19 | ENSRNOG00000000907 |
| 10925158 | 4.34E−19 | NA |
| 10845708 | 4.40E−19 | ENSRNOG00000006227 |
| 10750524 | 4.50E−19 | ENSRNOG00000001963 |
| 10860597 | 4.73E−19 | ENSRNOG00000008602 |
| 10840448 | 4.93E−19 | ENSRNOG00000010131 |
| 10715301 | 5.42E−19 | ENSRNOG00000014551 |
| 10940647 | 5.49E−19 | NA |
| 10773961 | 5.66E−19 | ENSRNOG00000010298 |
| 10794116 | 6.53E−19 | ENSRNOG00000026235 |
| 10841693 | 7.26E−19 | ENSRNOG00000014532 |
| 10931017 | 8.58E−19 | NA |
| 10750277 | 8.65E−19 | ENSRNOG00000002010 |
| 10774171 | 9.28E−19 | ENSRNOG00000004972 |
| 10883903 | 9.50E−19 | ENSRNOG00000023272 |
| 10814331 | 9.52E−19 | ENSRNOG00000011441 |
| 10778028 | 9.63E−19 | ENSRNOG00000004280 |
| 10910619 | 9.88E−19 | ENSRNOG00000012868 |
| 10851560 | 9.96E−19 | ENSRNOG00000028779 |
| 10926277 | 1.09E−18 | ENSRNOG00000022859 |
| 10873355 | 1.11E−18 | ENSRNOG00000017401 |
| 10754592 | 1.45E−18 | ENSRNOG00000001793 |
| 10906373 | 1.47E−18 | ENSRNOG00000026128 |
| 10901409 | 1.49E−18 | ENSRNOG00000008885 |
| 10762254 | 1.53E−18 | ENSRNOG00000033220 |
| 10846286 | 1.58E−18 | ENSRNOG00000001548 |
| 10901413 | 1.59E−18 | ENSRNOG00000009088 |
| 10755544 | 1.61E−18 | ENSRNOG00000001907 |
| 10931034 | 1.72E−18 | NA |
| 10932037 | 1.80E−18 | ENSRNOG00000009746 |
| 10783933 | 1.81E−18 | ENSRNOG00000020401 |
| 10839320 | 1.93E−18 | ENSRNOG00000000172 |
| 10812354 | 1.98E−18 | ENSRNOG00000032789 |
| 10779006 | 2.16E−18 | ENSRNOG00000013112 |
| 10897428 | 2.21E−18 | ENSRNOG00000000187 |
| 10923470 | 2.75E−18 | ENSRNOG00000011771 |
| 10750282 | 2.91E−18 | ENSRNOG00000027076 |
| 10809204 | 2.98E−18 | ENSRNOG00000014896 |
| 10753959 | 3.24E−18 | ENSRNOG00000030246 |
| 10828778 | 3.29E−18 | ENSRNOG00000000515 |
| 10851581 | 3.39E−18 | ENSRNOG00000030539 |
| 10751871 | 3.46E−18 | ENSRNOG00000001931 |
| 10891352 | 3.50E−18 | ENSRNOG00000012210 |
| 10940686 | 3.80E−18 | NA |
| 10773148 | 3.83E−18 | ENSRNOG00000005261 |
| 10796440 | 4.45E−18 | ENSRNOG00000017328 |
| 10863430 | 4.57E−18 | ENSRNOG00000006116 |
| 10756178 | 4.60E−18 | ENSRNOG00000001005 |
| 10845814 | 4.60E−18 | ENSRNOG00000005727 |
| 10794866 | 4.68E−18 | ENSRNOG00000016581 |
| 10718351 | 5.16E−18 | ENSRNOG00000011174 |
| 10726550 | 5.37E−18 | ENSRNOG00000017897 |
| 10911315 | 5.54E−18 | ENSRNOG00000011886 |
| 10709860 | 6.31E−18 | ENSRNOG00000009910 |
| 10940619 | 6.40E−18 | NA |
| 10940620 | 7.11E−18 | NA |
| 10764722 | 7.37E−18 | ENSRNOG00000028016 |
| 10924245 | 7.72E−18 | ENSRNOG00000014269 |
| 10916016 | 8.48E−18 | ENSRNOG00000009850 |
| 10907924 | 9.10E−18 | ENSRNOG00000040208 |
| 10826691 | 9.73E−18 | ENSRNOG00000024492 |
| 10774383 | 9.87E−18 | ENSRNOG00000007125 |
| 10871957 | 1.04E−17 | ENSRNOG00000008759 |
| 10912218 | 1.06E−17 | ENSRNOG00000008048 |
| 10820223 | 1.12E−17 | ENSRNOG00000033134 |
| 10703508 | 1.13E−17 | ENSRNOG00000030444 |
| 10722992 | 1.19E−17 | ENSRNOG00000014610 |
| 10900735 | 1.26E−17 | ENSRNOG00000033564 |
| 10825443 | 1.28E−17 | ENSRNOG00000037190 |
| 10754363 | 1.32E−17 | ENSRNOG00000032728 |
| 10898315 | 1.33E−17 | ENSRNOG00000014137 |
| 10922882 | 1.38E−17 | ENSRNOG00000015062 |
| 10801821 | 1.45E−17 | ENSRNOG00000013774 |
| 10940628 | 1.51E−17 | NA |
| 10852636 | 1.52E−17 | NA |
| 10733849 | 1.53E−17 | ENSRNOG00000002820 |
| 10920860 | 1.54E−17 | ENSRNOG00000013634 |
| 10788627 | 1.83E−17 | ENSRNOG00000010392 |
| 10766998 | 1.83E−17 | ENSRNOG00000030444 |
| 10782668 | 2.16E−17 | ENSRNOG00000008024 |
| 10746293 | 2.29E−17 | ENSRNOG00000002948 |
| 10821383 | 2.38E−17 | ENSRNOG00000010971 |
| 10721268 | 2.42E−17 | ENSRNOG00000022640 |
| 10852200 | 2.55E−17 | ENSRNOG00000004529 |
| 10858581 | 2.65E−17 | ENSRNOG00000027655 |
| 10854737 | 2.79E−17 | NA |
| 10703751 | 3.79E−17 | ENSRNOG00000038776 |
| 10905689 | 3.79E−17 | ENSRNOG00000007664 |
| 10880960 | 3.85E−17 | ENSRNOG00000006855 |
| 10772770 | 4.20E−17 | NA |
| 10712171 | 4.25E−17 | ENSRNOG00000004273 |
| 10739913 | 4.91E−17 | NA |
| 10858390 | 4.94E−17 | ENSRNOG00000013547 |
| 10711260 | 5.39E−17 | ENSRNOG00000022099 |
| 10834502 | 5.60E−17 | ENSRNOG00000009239 |
| 10866850 | 5.70E−17 | ENSRNOG00000009338 |
| 10717620 | 5.70E−17 | ENSRNOG00000018960 |
| 10826954 | 5.79E−17 | NA |
| 10767716 | 6.11E−17 | ENSRNOG00000028250 |
| 10940529 | 6.41E−17 | NA |
| 10795646 | 6.48E−17 | ENSRNOG00000018972 |
| 10858648 | 6.98E−17 | ENSRNOG00000011718 |
| 10739353 | 7.74E−17 | ENSRNOG00000004720 |
| 10910641 | 8.30E−17 | ENSRNOG00000013013 |
| 10709880 | 8.69E−17 | ENSRNOG00000018262 |
| 10922428 | 9.01E−17 | ENSRNOG00000013415 |
| 10723866 | 9.07E−17 | ENSRNOG00000016573 |
| 10894616 | 9.88E−17 | ENSRNOG00000039003 |
| 10771012 | 1.04E−16 | ENSRNOG00000023533 |
| 10747330 | 1.04E−16 | ENSRNOG00000015380 |
| 10901962 | 1.10E−16 | ENSRNOG00000007970 |
| 10722317 | 1.12E−16 | ENSRNOG00000012690 |
| 10893258 | 1.15E−16 | ENSRNOG00000007650 |
| 10703666 | 1.21E−16 | ENSRNOG00000011236 |
| 10718934 | 1.22E−16 | ENSRNOG00000027855 |
| 10733982 | 1.22E−16 | ENSRNOG00000003170 |
| 10785590 | 1.24E−16 | ENSRNOG00000009145 |
| 10772768 | 1.24E−16 | ENSRNOG00000002161 |
| 10751431 | 1.30E−16 | ENSRNOG00000033929 |
| 10798420 | 1.37E−16 | ENSRNOG00000003094 |
| 10831077 | 1.39E−16 | ENSRNOG00000000827 |
| 10903971 | 1.41E−16 | ENSRNOG00000029292 |
| 10776888 | 1.43E−16 | ENSRNOG00000026504 |
| 10865381 | 1.49E−16 | ENSRNOG00000010203 |
| 10849125 | 1.62E−16 | ENSRNOG00000022868 |
| 10814480 | 1.68E−16 | ENSRNOG00000011119 |
| 10765195 | 1.72E−16 | ENSRNOG00000002794 |
| 10826967 | 1.75E−16 | NA |
| 10816144 | 1.80E−16 | ENSRNOG00000009465 |
| 10756161 | 1.83E−16 | ENSRNOG00000040128 |
| 10751091 | 1.84E−16 | ENSRNOG00000002141 |
| 10864840 | 1.95E−16 | ENSRNOG00000011614 |
| 10870046 | 2.00E−16 | ENSRNOG00000007551 |
| 10858599 | 2.02E−16 | ENSRNOG00000010181 |
| 10818326 | 2.02E−16 | ENSRNOG00000031814 |
| 10779002 | 2.07E−16 | ENSRNOG00000033011 |
| 10869487 | 2.18E−16 | ENSRNOG00000008712 |
| 10938219 | 2.19E−16 | ENSRNOG00000034116 |
| 10704665 | 2.28E−16 | ENSRNOG00000013395 |
| 10826682 | 2.30E−16 | ENSRNOG00000022636 |
| 10769735 | 2.30E−16 | ENSRNOG00000024632 |
| 10929319 | 2.41E−16 | ENSRNOG00000018972 |
| 10764079 | 2.77E−16 | ENSRNOG00000003336 |
| 10754995 | 2.82E−16 | ENSRNOG00000001930 |
| 10744687 | 3.01E−16 | ENSRNOG00000014870 |
| 10828344 | 3.05E−16 | ENSRNOG00000032844 |
| 10794225 | 3.16E−16 | ENSRNOG00000011668 |
| 10801884 | 3.31E−16 | ENSRNOG00000015971 |
| 10820282 | 3.64E−16 | ENSRNOG00000029212 |
| 10875680 | 3.74E−16 | ENSRNOG00000006298 |
| 10860327 | 3.75E−16 | ENSRNOG00000006526 |
| 10759604 | 4.05E−16 | ENSRNOG00000028259 |
| 10863726 | 4.55E−16 | ENSRNOG00000017720 |
| 10752262 | 4.64E−16 | ENSRNOG00000030506 |
| 10736486 | 5.10E−16 | ENSRNOG00000003873 |
| 10829116 | 5.12E−16 | ENSRNOG00000001169 |
| 10837621 | 5.18E−16 | ENSRNOG00000026128 |
| 10826958 | 5.36E−16 | ENSRNOG00000032247 |
| 10700478 | 5.45E−16 | NA |
| 10731922 | 5.60E−16 | ENSRNOG00000008134 |
| 10730636 | 5.60E−16 | ENSRNOG00000020325 |
| 10817370 | 5.75E−16 | ENSRNOG00000021131 |
| 10864512 | 5.86E−16 | ENSRNOG00000005954 |
| 10796149 | 5.96E−16 | ENSRNOG00000018911 |
| 10940618 | 5.97E−16 | ENSRNOG00000042912 |
| 10799461 | 6.10E−16 | ENSRNOG00000029235 |
| 10720790 | 6.45E−16 | ENSRNOG00000021021 |
| 10788427 | 6.55E−16 | ENSRNOG00000012779 |
| 10758351 | 6.72E−16 | ENSRNOG00000026653 |
| 10872935 | 6.73E−16 | ENSRNOG00000009260 |
| 10903977 | 6.76E−16 | ENSRNOG00000028190 |
| 10901910 | 7.02E−16 | ENSRNOG00000005266 |
| 10701934 | 7.15E−16 | ENSRNOG00000012259 |
| 10857950 | 7.46E−16 | ENSRNOG00000007486 |
| 10818594 | 7.68E−16 | ENSRNOG00000015029 |
| 10716896 | 7.74E−16 | ENSRNOG00000015473 |
| 10909761 | 7.78E−16 | ENSRNOG00000018778 |
| 10909590 | 7.88E−16 | ENSRNOG00000016085 |
| 10782511 | 8.04E−16 | ENSRNOG00000005883 |
| 10878674 | 8.32E−16 | ENSRNOG00000033593 |
| 10834719 | 8.56E−16 | ENSRNOG00000009446 |
| 10746968 | 9.04E−16 | ENSRNOG00000036862 |
| 10929211 | 9.26E−16 | ENSRNOG00000013213 |
| 10714273 | 9.45E−16 | ENSRNOG00000025644 |
| 10829993 | 1.01E−15 | ENSRNOG00000000569 |
| 10854832 | 1.06E−15 | ENSRNOG00000026177 |
| 10842239 | 1.07E−15 | ENSRNOG00000017539 |
| 10864404 | 1.12E−15 | ENSRNOG00000009184 |
| 10840890 | 1.14E−15 | ENSRNOG00000021750 |
| 10936365 | 1.15E−15 | ENSRNOG00000013170 |
| 10816278 | 1.16E−15 | ENSRNOG00000034230 |
| 10733056 | 1.19E−15 | ENSRNOG00000002470 |
| 10754983 | 1.23E−15 | ENSRNOG00000013170 |
| 10744545 | 1.24E−15 | ENSRNOG00000037409 |
| 10813361 | 1.30E−15 | ENSRNOG00000028930 |
| 10768742 | 1.36E−15 | ENSRNOG00000026952 |
| 10788374 | 1.45E−15 | ENSRNOG00000010748 |
| 10763758 | 1.52E−15 | ENSRNOG00000004405 |
| 10895589 | 1.56E−15 | ENSRNOG00000034134 |
| 10706346 | 1.62E−15 | ENSRNOG00000023411 |
| 10924803 | 1.64E−15 | ENSRNOG00000017256 |
| 10914799 | 1.67E−15 | ENSRNOG00000005731 |
| 10716303 | 1.68E−15 | ENSRNOG00000017225 |
| 10889965 | 1.77E−15 | ENSRNOG00000006828 |
| 10842707 | 1.84E−15 | ENSRNOG00000024702 |
| 10848450 | 1.84E−15 | ENSRNOG00000008229 |
| 10770778 | 1.86E−15 | ENSRNOG00000005565 |
| 10819644 | 1.93E−15 | ENSRNOG00000014613 |
| 10725953 | 1.98E−15 | ENSRNOG00000036711 |
| 10824695 | 2.04E−15 | ENSRNOG00000011483 |
| 10888610 | 2.17E−15 | ENSRNOG00000039902 |
| 10794242 | 2.31E−15 | ENSRNOG00000013679 |
| 10905335 | 2.39E−15 | ENSRNOG00000008401 |
| 10802013 | 2.40E−15 | ENSRNOG00000018735 |
| 10854847 | 2.59E−15 | ENSRNOG00000026177 |
| 10927562 | 2.81E−15 | ENSRNOG00000018830 |
| 10728240 | 2.85E−15 | ENSRNOG00000021125 |
| 10886036 | 2.86E−15 | ENSRNOG00000008224 |
| 10716746 | 3.04E−15 | NA |
| 10705034 | 3.30E−15 | ENSRNOG00000020090 |
| 10750460 | 3.36E−15 | ENSRNOG00000001647 |
| 10766726 | 3.39E−15 | ENSRNOG00000003915 |
| 10811956 | 3.41E−15 | ENSRNOG00000019791 |
| 10864848 | 3.51E−15 | ENSRNOG00000012972 |
| 10878617 | 3.55E−15 | ENSRNOG00000010859 |
| 10807140 | 3.68E−15 | ENSRNOG00000014668 |
| 10709135 | 3.84E−15 | ENSRNOG00000019319 |
| 10922826 | 3.90E−15 | ENSRNOG00000014504 |
| 10708521 | 3.94E−15 | ENSRNOG00000016496 |
| 10801308 | 4.25E−15 | ENSRNOG00000013920 |
| 10818920 | 4.33E−15 | ENSRNOG00000011589 |
| 10735182 | 4.36E−15 | ENSRNOG00000018715 |
| 10766760 | 4.36E−15 | ENSRNOG00000004402 |
| 10759717 | 4.78E−15 | ENSRNOG00000000973 |
| 10796445 | 4.80E−15 | ENSRNOG00000018087 |
| 10807452 | 4.93E−15 | ENSRNOG00000018618 |
| 10826672 | 4.98E−15 | ENSRNOG00000022636 |
| 10700829 | 5.19E−15 | NA |
| 10794084 | 5.39E−15 | ENSRNOG00000016539 |
| 10802603 | 5.40E−15 | ENSRNOG00000014614 |
| 10791935 | 5.46E−15 | ENSRNOG00000022686 |
| 10849275 | 5.46E−15 | ENSRNOG00000028668 |
| 10770497 | 6.28E−15 | ENSRNOG00000002309 |
| 10737663 | 6.67E−15 | ENSRNOG00000006108 |
| 10732939 | 6.78E−15 | ENSRNOG00000038963 |
| 10772657 | 7.07E−15 | ENSRNOG00000002408 |
| 10804017 | 7.08E−15 | ENSRNOG00000019688 |
| 10823970 | 7.38E−15 | ENSRNOG00000009822 |
| 10892143 | 7.63E−15 | ENSRNOG00000011716 |
| 10736163 | 7.86E−15 | ENSRNOG00000009681 |
| 10921163 | 7.86E−15 | ENSRNOG00000006715 |
| 10904593 | 8.11E−15 | ENSRNOG00000037375 |
| 10836446 | 8.26E−15 | ENSRNOG00000007359 |
| 10909948 | 8.35E−15 | ENSRNOG00000011500 |
| 10838605 | 8.64E−15 | ENSRNOG00000006027 |
| 10714903 | 9.16E−15 | ENSRNOG00000022839 |
| 10731047 | 9.82E−15 | ENSRNOG00000013687 |
| 10702829 | 9.94E−15 | ENSRNOG00000017114 |
| 10769797 | 1.10E−14 | ENSRNOG00000003138 |
| 10914385 | 1.13E−14 | ENSRNOG00000019262 |
| 10722743 | 1.16E−14 | ENSRNOG00000009932 |
| 10932475 | 1.18E−14 | ENSRNOG00000006791 |
| 10782704 | 1.20E−14 | ENSRNOG00000009470 |
| 10852244 | 1.21E−14 | ENSRNOG00000024915 |
| 10907793 | 1.24E−14 | NA |
| 10864713 | 1.25E−14 | NA |
| 10726687 | 1.34E−14 | ENSRNOG00000036645 |
| 10912088 | 1.34E−14 | ENSRNOG00000011071 |
| 10815857 | 1.35E−14 | ENSRNOG00000009276 |
| 10770858 | 1.35E−14 | ENSRNOG00000034164 |
| 10909603 | 1.39E−14 | ENSRNOG00000026702 |
| 10866417 | 1.40E−14 | ENSRNOG00000000024 |
| 10737047 | 1.45E−14 | ENSRNOG00000002916 |
| 10854239 | 1.46E−14 | ENSRNOG00000028742 |
| 10885740 | 1.47E−14 | ENSRNOG00000007646 |
| 10735436 | 1.47E−14 | ENSRNOG00000037371 |
| 10789591 | 1.51E−14 | ENSRNOG00000023991 |
| 10907913 | 1.54E−14 | ENSRNOG00000009907 |
| 10926683 | 1.55E−14 | ENSRNOG00000025691 |
| 10764781 | 1.60E−14 | ENSRNOG00000027017 |
| 10726679 | 1.60E−14 | ENSRNOG00000014936 |
| 10736135 | 1.65E−14 | ENSRNOG00000009673 |
| 10867045 | 1.69E−14 | ENSRNOG00000031045 |
| 10822583 | 1.80E−14 | ENSRNOG00000024089 |
| 10768754 | 1.80E−14 | ENSRNOG00000026952 |
| 10834213 | 1.84E−14 | ENSRNOG00000013102 |
| 10718942 | 1.91E−14 | ENSRNOG00000018600 |
| 10792863 | 2.01E−14 | ENSRNOG00000017154 |
| 10767723 | 2.05E−14 | ENSRNOG00000003031 |
| 10911811 | 2.14E−14 | ENSRNOG00000044467 |
| 10717331 | 2.30E−14 | ENSRNOG00000011815 |
| 10834022 | 2.54E−14 | NA |
| 10930428 | 2.57E−14 | ENSRNOG00000014837 |
| 10718602 | 2.59E−14 | ENSRNOG00000035545 |
| 10826607 | 2.71E−14 | ENSRNOG00000009345 |
| 10896793 | 2.79E−14 | ENSRNOG00000004100 |
| 10721388 | 2.91E−14 | ENSRNOG00000019660 |
| 10734342 | 2.92E−14 | ENSRNOG00000003214 |
| 10924151 | 3.12E−14 | ENSRNOG00000015511 |
| 10839974 | 3.16E−14 | ENSRNOG00000021240 |
| 10940595 | 3.18E−14 | NA |
| 10914042 | 3.20E−14 | ENSRNOG00000037121 |
| 10701830 | 3.22E−14 | ENSRNOG00000011015 |
| 10782493 | 3.48E−14 | ENSRNOG00000005883 |
| 10710541 | 3.67E−14 | ENSRNOG00000025940 |
| 10725618 | 3.70E−14 | ENSRNOG00000016722 |
| 10919494 | 3.72E−14 | ENSRNOG00000014060 |
| 10716785 | 3.73E−14 | ENSRNOG00000014258 |
| 10764069 | 3.91E−14 | ENSRNOG00000003312 |
| 10710063 | 4.02E−14 | ENSRNOG00000025393 |
| 10719777 | 4.06E−14 | ENSRNOG00000020381 |
| 10748118 | 4.17E−14 | ENSRNOG00000003905 |
| 10762247 | 4.23E−14 | ENSRNOG00000001372 |
| 10815317 | 4.40E−14 | ENSRNOG00000013397 |
| 10786174 | 4.51E−14 | ENSRNOG00000010984 |
| 10768979 | 4.63E−14 | ENSRNOG00000004111 |
| 10831557 | 4.70E−14 | ENSRNOG00000033215 |
| 10891039 | 4.70E−14 | ENSRNOG00000009653 |
| 10812216 | 4.88E−14 | ENSRNOG00000012183 |
| 10753425 | 4.92E−14 | ENSRNOG00000001959 |
| 10701358 | 5.03E−14 | NA |
| 10781829 | 5.12E−14 | ENSRNOG00000008785 |
| 10809824 | 5.27E−14 | ENSRNOG00000016841 |
| 10782509 | 5.74E−14 | ENSRNOG00000005883 |
| 10940554 | 5.83E−14 | NA |
| 10797811 | 5.96E−14 | ENSRNOG00000018092 |
| 10857546 | 5.98E−14 | ENSRNOG00000007104 |
| 10902492 | 6.10E−14 | ENSRNOG00000005362 |
| 10714276 | 6.30E−14 | ENSRNOG00000013053 |
| 10916804 | 6.86E−14 | ENSRNOG00000027457 |
| 10852136 | 7.00E−14 | ENSRNOG00000006314 |
| 10818633 | 7.06E−14 | ENSRNOG00000016351 |
| 10863639 | 7.38E−14 | ENSRNOG00000037871 |
| 10706308 | 7.84E−14 | ENSRNOG00000037339 |
| 10926930 | 7.95E−14 | ENSRNOG00000012543 |
| 10855075 | 8.00E−14 | ENSRNOG00000026817 |
| 10726682 | 8.05E−14 | ENSRNOG00000015078 |
| 10926405 | 8.13E−14 | ENSRNOG00000018741 |
| 10715258 | 8.15E−14 | ENSRNOG00000014574 |
| 10896380 | 8.27E−14 | ENSRNOG00000005123 |
| 10714900 | 8.95E−14 | ENSRNOG00000036604 |
| 10719364 | 8.95E−14 | ENSRNOG00000013767 |
| 10802619 | 8.99E−14 | NA |
| 10855079 | 9.04E−14 | ENSRNOG00000017881 |
| 10779839 | 9.44E−14 | ENSRNOG00000025597 |
| 10931222 | 9.95E−14 | NA |
| 10727067 | 1.01E−13 | ENSRNOG00000020615 |
| 10719847 | 1.02E−13 | ENSRNOG00000020578 |
| 10816901 | 1.03E−13 | ENSRNOG00000017070 |
| 10880408 | 1.04E−13 | ENSRNOG00000033313 |
| 10866091 | 1.06E−13 | ENSRNOG00000025346 |
| 10832808 | 1.09E−13 | ENSRNOG00000000648 |
| 10848393 | 1.09E−13 | ENSRNOG00000005404 |
| 10798108 | 1.09E−13 | ENSRNOG00000017787 |
| 10862131 | 1.14E−13 | ENSRNOG00000026306 |
| 10928855 | 1.14E−13 | ENSRNOG00000014182 |
| 10862031 | 1.14E−13 | ENSRNOG00000008725 |
| 10712317 | 1.15E−13 | ENSRNOG00000018736 |
| 10920390 | 1.19E−13 | ENSRNOG00000020733 |
| 10820098 | 1.21E−13 | ENSRNOG00000040283 |
| 10877630 | 1.24E−13 | ENSRNOG00000005932 |
| 10771535 | 1.27E−13 | ENSRNOG00000000081 |
| 10718600 | 1.29E−13 | ENSRNOG00000035460 |
| 10886756 | 1.34E−13 | ENSRNOG00000014476 |
| 10753279 | 1.34E−13 | ENSRNOG00000001652 |
| 10829313 | 1.37E−13 | ENSRNOG00000001216 |
| 10927041 | 1.44E−13 | ENSRNOG00000014142 |
| 10827231 | 1.45E−13 | ENSRNOG00000014350 |
| 10899055 | 1.47E−13 | ENSRNOG00000015544 |
| 10926091 | 1.47E−13 | ENSRNOG00000029305 |
| 10786640 | 1.48E−13 | ENSRNOG00000018943 |
| 10907763 | 1.48E−13 | NA |
| 10753784 | 1.49E−13 | ENSRNOG00000001958 |
| 10890886 | 1.51E−13 | ENSRNOG00000030024 |
| 10749797 | 1.53E−13 | ENSRNOG00000036662 |
| 10921166 | 1.55E−13 | ENSRNOG00000006730 |
| 10918569 | 1.56E−13 | ENSRNOG00000015949 |
| 10890441 | 1.62E−13 | ENSRNOG00000006388 |
| 10794328 | 1.62E−13 | ENSRNOG00000016225 |
| 10869483 | 1.67E−13 | ENSRNOG00000008163 |
| 10940661 | 1.68E−13 | ENSRNOG00000042848 |
| 10830253 | 1.70E−13 | ENSRNOG00000000824 |
| 10812775 | 1.73E−13 | ENSRNOG00000010266 |
| 10729269 | 1.76E−13 | ENSRNOG00000017469 |
| 10703662 | 1.77E−13 | NA |
| 10743357 | 1.90E−13 | ENSRNOG00000012258 |
| 10919010 | 1.90E−13 | ENSRNOG00000031032 |
| 10919637 | 1.92E−13 | ENSRNOG00000030625 |
| 10701846 | 1.98E−13 | ENSRNOG00000012193 |
| 10868923 | 2.00E−13 | ENSRNOG00000007036 |
| 10705731 | 2.04E−13 | ENSRNOG00000023931 |
| 10926967 | 2.17E−13 | ENSRNOG00000033402 |
| 10724042 | 2.22E−13 | ENSRNOG00000019283 |
| 10765339 | 2.23E−13 | ENSRNOG00000003298 |
| 10727084 | 2.24E−13 | ENSRNOG00000020651 |
| 10831060 | 2.25E−13 | ENSRNOG00000000826 |
| 10882273 | 2.28E−13 | ENSRNOG00000020205 |
| 10750876 | 2.29E−13 | ENSRNOG00000013167 |
| 10809912 | 2.32E−13 | ENSRNOG00000023910 |
| 10917116 | 2.33E−13 | ENSRNOG00000029980 |
| 10788720 | 2.34E−13 | ENSRNOG00000012688 |
| 10794800 | 2.41E−13 | ENSRNOG00000017093 |
| 10940594 | 2.47E−13 | NA |
| 10924769 | 2.60E−13 | ENSRNOG00000015619 |
| 10787960 | 2.65E−13 | ENSRNOG00000033313 |
| 10729209 | 2.76E−13 | NA |
| 10816204 | 2.80E−13 | ENSRNOG00000023453 |
| 10764661 | 2.85E−13 | ENSRNOG00000002403 |
| 10934635 | 3.35E−13 | ENSRNOG00000037839 |
| 10752754 | 3.36E−13 | ENSRNOG00000001585 |
| 10862867 | 3.45E−13 | ENSRNOG00000005615 |
| 10862283 | 3.46E−13 | ENSRNOG00000031457 |
| 10932016 | 3.74E−13 | ENSRNOG00000043182 |
| 10746905 | 4.00E−13 | ENSRNOG00000007200 |
| 10822830 | 4.20E−13 | ENSRNOG00000014414 |
| 10827796 | 4.21E−13 | ENSRNOG00000000795 |
| 10704996 | 4.23E−13 | ENSRNOG00000019982 |
| 10828714 | 4.31E−13 | ENSRNOG00000000503 |
| 10917385 | 4.33E−13 | ENSRNOG00000029773 |
| 10816153 | 4.34E−13 | ENSRNOG00000010866 |
| 10908896 | 4.54E−13 | ENSRNOG00000008941 |
| 10802621 | 4.59E−13 | ENSRNOG00000024104 |
| 10859337 | 4.63E−13 | ENSRNOG00000005947 |
| 10819052 | 4.90E−13 | ENSRNOG00000010121 |
| 10932164 | 4.96E−13 | ENSRNOG00000035581 |
| 10866413 | 4.97E−13 | ENSRNOG00000008439 |
| 10740442 | 5.12E−13 | ENSRNOG00000002659 |
| 10720797 | 5.26E−13 | ENSRNOG00000024000 |
| 10776778 | 5.48E−13 | ENSRNOG00000027152 |
| 10774163 | 5.52E−13 | ENSRNOG00000037632 |
| 10924177 | 5.60E−13 | ENSRNOG00000016105 |
| 10876838 | 5.75E−13 | ENSRNOG00000016299 |
| 10784412 | 5.76E−13 | ENSRNOG00000038799 |
| 10863777 | 6.16E−13 | ENSRNOG00000008678 |
| 10761965 | 6.18E−13 | ENSRNOG00000001336 |
| 10865349 | 6.18E−13 | ENSRNOG00000008376 |
| 10864942 | 6.23E−13 | ENSRNOG00000006576 |
| 10763935 | 6.32E−13 | ENSRNOG00000029938 |
| 10750211 | 6.38E−13 | ENSRNOG00000002032 |
| 10887239 | 6.45E−13 | ENSRNOG00000021904 |
| 10934639 | 6.63E−13 | ENSRNOG00000032970 |
| 10828351 | 6.83E−13 | ENSRNOG00000000451 |
| 10750672 | 6.92E−13 | ENSRNOG00000001653 |
| 10732966 | 7.10E−13 | ENSRNOG00000042980 |
| 10701924 | 7.12E−13 | ENSRNOG00000012074 |
| 10781960 | 7.40E−13 | NA |
| 10776026 | 7.58E−13 | ENSRNOG00000003296 |
| 10830624 | 7.63E−13 | ENSRNOG00000000321 |
| 10729460 | 7.63E−13 | NA |
| 10807688 | 8.21E−13 | ENSRNOG00000014452 |
| 10749484 | 8.57E−13 | ENSRNOG00000003148 |
| 10855062 | 8.75E−13 | ENSRNOG00000017354 |
| 10711268 | 8.85E−13 | ENSRNOG00000019728 |
| 10775628 | 9.14E−13 | ENSRNOG00000002045 |
| 10904597 | 9.16E−13 | ENSRNOG00000037374 |
| 10853819 | 9.20E−13 | ENSRNOG00000007079 |
| 10752744 | 9.53E−13 | ENSRNOG00000030930 |
| 10907122 | 9.62E−13 | ENSRNOG00000021503 |
| 10935255 | 1.00E−12 | ENSRNOG00000032878 |
| 10742348 | 1.00E−12 | ENSRNOG00000031138 |
| 10804935 | 1.01E−12 | ENSRNOG00000017421 |
| 10718598 | 1.05E−12 | ENSRNOG00000035495 |
| 10723092 | 1.06E−12 | ENSRNOG00000011683 |
| 10879483 | 1.06E−12 | ENSRNOG00000027921 |
| 10749757 | 1.13E−12 | ENSRNOG00000036674 |
| 10707386 | 1.14E−12 | ENSRNOG00000015415 |
| 10856265 | 1.15E−12 | ENSRNOG00000007129 |
| 10724575 | 1.16E−12 | NA |
| 10928275 | 1.17E−12 | ENSRNOG00000025079 |
| 10914011 | 1.20E−12 | ENSRNOG00000010951 |
| 10859392 | 1.21E−12 | ENSRNOG00000007743 |
| 10862262 | 1.24E−12 | ENSRNOG00000017525 |
| 10869165 | 1.24E−12 | ENSRNOG00000019021 |
| 10808085 | 1.25E−12 | ENSRNOG00000019028 |
| 10766953 | 1.30E−12 | ENSRNOG00000002791 |
| 10920556 | 1.31E−12 | ENSRNOG00000037167 |
| 10763149 | 1.36E−12 | NA |
| 10729793 | 1.39E−12 | ENSRNOG00000035954 |
| 10724993 | 1.42E−12 | ENSRNOG00000012258 |
| 10811751 | 1.48E−12 | ENSRNOG00000028904 |
| 10796476 | 1.51E−12 | ENSRNOG00000018251 |
| 10804486 | 1.65E−12 | ENSRNOG00000017035 |
| 10776774 | 1.68E−12 | ENSRNOG00000002540 |
| 10940690 | 1.68E−12 | NA |
| 10718311 | 1.75E−12 | ENSRNOG00000021493 |
| 10931717 | 1.76E−12 | NA |
| 10889339 | 1.76E−12 | ENSRNOG00000007503 |
| 10908452 | 1.76E−12 | ENSRNOG00000033256 |
| 10742494 | 1.79E−12 | ENSRNOG00000003475 |
| 10768412 | 1.84E−12 | ENSRNOG00000002385 |
| 10828229 | 1.90E−12 | ENSRNOG00000030729 |
| 10849700 | 1.93E−12 | ENSRNOG00000015445 |
| 10841489 | 2.01E−12 | ENSRNOG00000019948 |
| 10868017 | 2.03E−12 | NA |
| 10917366 | 2.05E−12 | ENSRNOG00000012500 |
| 10938384 | 2.07E−12 | ENSRNOG00000012513 |
| 10845767 | 2.07E−12 | ENSRNOG00000027016 |
| 10719593 | 2.11E−12 | NA |
| 10825153 | 2.17E−12 | ENSRNOG00000021199 |
| 10751498 | 2.22E−12 | ENSRNOG00000032327 |
| 10858967 | 2.26E−12 | ENSRNOG00000031312 |
| 10801973 | 2.48E−12 | ENSRNOG00000038960 |
| 10766170 | 2.50E−12 | ENSRNOG00000003084 |
| 10920770 | 2.50E−12 | ENSRNOG00000000219 |
| 10870490 | 2.54E−12 | ENSRNOG00000005802 |
| 10881293 | 2.60E−12 | ENSRNOG00000014961 |
| 10817071 | 2.64E−12 | ENSRNOG00000011557 |
| 10740408 | 2.66E−12 | NA |
| 10822330 | 2.67E−12 | ENSRNOG00000011319 |
| 10734673 | 2.68E−12 | ENSRNOG00000003492 |
| 10714020 | 2.81E−12 | ENSRNOG00000020991 |
| 10751086 | 2.84E−12 | ENSRNOG00000039088 |
| 10922113 | 2.86E−12 | NA |
| 10936827 | 2.88E−12 | ENSRNOG00000003858 |
| 10837794 | 3.00E−12 | ENSRNOG00000012172 |
| 10820145 | 3.13E−12 | ENSRNOG00000023651 |
| 10857984 | 3.23E−12 | ENSRNOG00000008839 |
| 10803991 | 3.23E−12 | ENSRNOG00000017819 |
| 10871103 | 3.31E−12 | ENSRNOG00000010091 |
| 10801606 | 3.39E−12 | ENSRNOG00000024671 |
| 10903979 | 3.39E−12 | ENSRNOG00000004744 |
| 10798199 | 3.48E−12 | ENSRNOG00000027125 |
| 10866819 | 3.62E−12 | ENSRNOG00000015514 |
| 10897419 | 3.66E−12 | ENSRNOG00000006940 |
| 10832831 | 3.75E−12 | ENSRNOG00000000648 |
| 10774596 | 3.81E−12 | ENSRNOG00000007049 |
| 10714353 | 3.86E−12 | ENSRNOG00000012782 |
| 10850257 | 3.88E−12 | ENSRNOG00000030601 |
| 10932139 | 3.97E−12 | ENSRNOG00000004150 |
| 10833180 | 4.14E−12 | ENSRNOG00000000394 |
| 10777242 | 4.19E−12 | ENSRNOG00000003064 |
| 10737740 | 4.23E−12 | ENSRNOG00000023881 |
| 10829703 | 4.41E−12 | ENSRNOG00000000613 |
| 10866019 | 4.45E−12 | ENSRNOG00000033879 |
| 10772729 | 4.46E−12 | ENSRNOG00000002855 |
| 10814561 | 4.49E−12 | ENSRNOG00000028156 |
| 10879134 | 4.49E−12 | ENSRNOG00000019977 |
| 10762740 | 4.51E−12 | ENSRNOG00000001187 |
| 10769016 | 4.64E−12 | ENSRNOG00000004736 |
| 10889772 | 4.72E−12 | ENSRNOG00000004198 |
| 10855077 | 4.74E−12 | ENSRNOG00000026811 |
| 10858702 | 4.75E−12 | ENSRNOG00000015125 |
| 10810631 | 4.77E−12 | ENSRNOG00000016890 |
| 10774605 | 4.88E−12 | ENSRNOG00000037522 |
| 10901436 | 4.92E−12 | ENSRNOG00000029645 |
| 10832829 | 4.95E−12 | ENSRNOG00000000648 |
| 10804086 | 4.99E−12 | ENSRNOG00000027025 |
| 10713222 | 5.08E−12 | ENSRNOG00000021028 |
| 10901289 | 5.31E−12 | NA |
| 10845470 | 5.50E−12 | ENSRNOG00000025148 |
| 10780919 | 5.53E−12 | ENSRNOG00000014653 |
| 10831574 | 5.62E−12 | ENSRNOG00000000454 |
| 10890860 | 5.72E−12 | ENSRNOG00000004309 |
| 10798027 | 5.91E−12 | ENSRNOG00000000137 |
| 10708587 | 6.08E−12 | NA |
| 10836019 | 6.14E−12 | ENSRNOG00000029993 |
| 10795574 | 6.48E−12 | ENSRNOG00000017791 |
| 10715546 | 6.48E−12 | ENSRNOG00000013279 |
| 10940575 | 6.75E−12 | NA |
| 10760920 | 7.01E−12 | ENSRNOG00000043372 |
| 10864838 | 7.14E−12 | NA |
| 10832680 | 7.15E−12 | ENSRNOG00000000609 |
| 10929536 | 7.16E−12 | ENSRNOG00000017072 |
| 10826680 | 7.19E−12 | ENSRNOG00000022636 |
| 10847903 | 7.34E−12 | ENSRNOG00000011358 |
| 10700735 | 7.34E−12 | NA |
| 10763375 | 7.35E−12 | ENSRNOG00000002417 |
| 10893067 | 7.59E−12 | ENSRNOG00000031081 |
| 10777770 | 7.59E−12 | ENSRNOG00000017259 |
| 10799516 | 7.88E−12 | ENSRNOG00000017644 |
| 10866167 | 8.08E−12 | ENSRNOG00000034136 |
| 10728766 | 8.18E−12 | ENSRNOG00000020872 |
| 10818983 | 8.21E−12 | ENSRNOG00000010941 |
| 10858784 | 8.24E−12 | ENSRNOG00000018309 |
| 10917518 | 8.28E−12 | ENSRNOG00000009170 |
| 10702250 | 8.65E−12 | ENSRNOG00000013994 |
| 10909470 | 8.83E−12 | ENSRNOG00000009799 |
| 10744460 | 8.83E−12 | ENSRNOG00000026647 |
| 10797572 | 8.88E−12 | ENSRNOG00000011119 |
| 10754426 | 9.58E−12 | ENSRNOG00000023463 |
| 10788899 | 9.67E−12 | ENSRNOG00000017231 |
| 10825495 | 9.81E−12 | ENSRNOG00000033051 |
| 10797929 | 9.89E−12 | ENSRNOG00000023778 |
| 10864711 | 9.99E−12 | ENSRNOG00000030444 |
| 10869249 | 1.05E−11 | ENSRNOG00000025040 |
| 10860820 | 1.06E−11 | ENSRNOG00000009711 |
| 10765497 | 1.07E−11 | ENSRNOG00000024382 |
| 10751896 | 1.09E−11 | ENSRNOG00000001926 |
| 10736784 | 1.11E−11 | NA |
| 10904653 | 1.13E−11 | ENSRNOG00000007939 |
| 10768697 | 1.15E−11 | ENSRNOG00000002735 |
| 10925185 | 1.17E−11 | ENSRNOG00000042182 |
| 10746819 | 1.20E−11 | ENSRNOG00000036880 |
| 10845508 | 1.21E−11 | ENSRNOG00000006623 |
| 10918620 | 1.21E−11 | ENSRNOG00000017472 |
| 10776835 | 1.22E−11 | ENSRNOG00000002932 |
| 10925753 | 1.23E−11 | ENSRNOG00000032398 |
| 10763889 | 1.24E−11 | ENSRNOG00000000034 |
| 10810867 | 1.24E−11 | ENSRNOG00000012772 |
| 10883609 | 1.26E−11 | ENSRNOG00000004908 |
| 10732207 | 1.26E−11 | ENSRNOG00000039510 |
| 10919389 | 1.27E−11 | ENSRNOG00000011909 |
| 10740381 | 1.29E−11 | ENSRNOG00000002294 |
| 10787162 | 1.30E−11 | ENSRNOG00000013502 |
| 10803001 | 1.31E−11 | NA |
| 10772342 | 1.32E−11 | ENSRNOG00000002248 |
| 10784227 | 1.33E−11 | ENSRNOG00000011580 |
| 10762747 | 1.33E−11 | ENSRNOG00000028814 |
| 10771492 | 1.34E−11 | ENSRNOG00000002345 |
| 10757862 | 1.37E−11 | ENSRNOG00000001479 |
| 10908521 | 1.37E−11 | ENSRNOG00000009946 |
| 10865585 | 1.38E−11 | ENSRNOG00000016294 |
| 10800814 | 1.39E−11 | ENSRNOG00000020478 |
| 10797597 | 1.43E−11 | ENSRNOG00000014616 |
| 10900974 | 1.46E−11 | ENSRNOG00000006559 |
| 10859880 | 1.46E−11 | ENSRNOG00000006859 |
| 10823783 | 1.47E−11 | ENSRNOG00000027833 |
| 10770396 | 1.47E−11 | ENSRNOG00000003103 |
| 10898192 | 1.50E−11 | ENSRNOG00000010549 |
| 10909918 | 1.50E−11 | ENSRNOG00000010922 |
| 10879555 | 1.51E−11 | ENSRNOG00000016047 |
| 10855512 | 1.51E−11 | ENSRNOG00000021579 |
| 10751931 | 1.57E−11 | ENSRNOG00000001843 |
| 10882709 | 1.59E−11 | ENSRNOG00000015603 |
| 10914571 | 1.63E−11 | ENSRNOG00000004837 |
| 10716335 | 1.63E−11 | ENSRNOG00000017406 |
| 10888662 | 1.64E−11 | ENSRNOG00000026316 |
| 10905530 | 1.68E−11 | ENSRNOG00000016896 |
| 10862281 | 1.69E−11 | ENSRNOG00000028934 |
| 10842315 | 1.70E−11 | ENSRNOG00000005616 |
| 10884772 | 1.73E−11 | ENSRNOG00000005101 |
| 10730659 | 1.80E−11 | NA |
| 10729780 | 1.80E−11 | ENSRNOG00000019077 |
| 10837366 | 1.81E−11 | ENSRNOG00000008347 |
| 10872561 | 1.84E−11 | ENSRNOG00000007021 |
| 10787491 | 1.90E−11 | ENSRNOG00000019387 |
| 10806806 | 1.94E−11 | ENSRNOG00000004489 |
| 10847308 | 1.97E−11 | ENSRNOG00000012568 |
| 10747024 | 1.98E−11 | ENSRNOG00000010665 |
| 10897054 | 1.98E−11 | ENSRNOG00000007091 |
| 10752244 | 2.00E−11 | NA |
| 10745424 | 2.02E−11 | ENSRNOG00000014285 |
| 10781467 | 2.05E−11 | ENSRNOG00000010063 |
| 10756877 | 2.11E−11 | ENSRNOG00000024277 |
| 10769782 | 2.16E−11 | ENSRNOG00000031708 |
| 10883636 | 2.16E−11 | ENSRNOG00000005836 |
| 10770685 | 2.26E−11 | ENSRNOG00000003767 |
| 10801650 | 2.29E−11 | ENSRNOG00000026136 |
| 10781496 | 2.36E−11 | ENSRNOG00000010319 |
| 10743966 | 2.37E−11 | ENSRNOG00000037613 |
| 10781722 | 2.40E−11 | ENSRNOG00000013280 |
| 10774345 | 2.41E−11 | ENSRNOG00000004888 |
| 10815636 | 2.41E−11 | ENSRNOG00000014076 |
| 10717891 | 2.58E−11 | ENSRNOG00000018524 |
| 10933755 | 2.64E−11 | ENSRNOG00000003686 |
| 10882317 | 2.64E−11 | ENSRNOG00000021802 |
| 10753861 | 2.69E−11 | ENSRNOG00000001943 |
| 10817686 | 2.81E−11 | ENSRNOG00000018076 |
| 10940677 | 2.82E−11 | NA |
| 10796564 | 2.83E−11 | ENSRNOG00000000142 |
| 10842475 | 2.84E−11 | ENSRNOG00000010574 |
| 10743914 | 2.88E−11 | ENSRNOG00000009722 |
| 10892950 | 2.90E−11 | ENSRNOG00000002632 |
| 10744568 | 2.93E−11 | ENSRNOG00000006949 |
| 10887939 | 3.02E−11 | ENSRNOG00000032136 |
| 10764358 | 3.08E−11 | ENSRNOG00000009099 |
| 10784049 | 3.08E−11 | ENSRNOG00000031868 |
| 10729074 | 3.10E−11 | ENSRNOG00000013239 |
| 10756154 | 3.14E−11 | ENSRNOG00000028803 |
| 10731891 | 3.14E−11 | ENSRNOG00000024210 |
| 10882933 | 3.19E−11 | ENSRNOG00000007081 |
| 10851263 | 3.19E−11 | ENSRNOG00000006418 |
| 10927678 | 3.30E−11 | ENSRNOG00000014317 |
| 10930411 | 3.33E−11 | ENSRNOG00000015906 |
| 10901823 | 3.34E−11 | ENSRNOG00000008797 |
| 10871521 | 3.42E−11 | ENSRNOG00000007284 |
| 10830216 | 3.44E−11 | ENSRNOG00000000811 |
| 10808362 | 3.46E−11 | ENSRNOG00000016509 |
| 10705712 | 3.47E−11 | ENSRNOG00000033744 |
| 10710051 | 3.49E−11 | ENSRNOG00000013176 |
| 10888608 | 3.54E−11 | ENSRNOG00000039902 |
| 10856320 | 3.56E−11 | ENSRNOG00000009545 |
| 10831090 | 3.68E−11 | ENSRNOG00000029682 |
| 10835355 | 3.72E−11 | ENSRNOG00000008837 |
| 10826846 | 3.72E−11 | ENSRNOG00000011284 |
| 10940630 | 3.75E−11 | NA |
| 10924286 | 3.90E−11 | ENSRNOG00000014956 |
| 10933733 | 3.93E−11 | ENSRNOG00000004402 |
| 10898676 | 3.99E−11 | ENSRNOG00000004048 |
| 10716667 | 4.11E−11 | ENSRNOG00000016381 |
| 10700744 | 4.19E−11 | NA |
| 10830398 | 4.29E−11 | ENSRNOG00000000595 |
| 10940494 | 4.30E−11 | NA |
| 10846185 | 4.31E−11 | ENSRNOG00000018485 |
| 10733790 | 4.48E−11 | ENSRNOG00000027008 |
| 10750193 | 4.52E−11 | ENSRNOG00000028638 |
| 10796615 | 4.67E−11 | ENSRNOG00000016585 |
| 10881671 | 4.69E−11 | ENSRNOG00000030317 |
| 10889649 | 4.73E−11 | ENSRNOG00000004174 |
| 10889064 | 4.77E−11 | ENSRNOG00000010667 |
| 10803681 | 4.78E−11 | ENSRNOG00000020468 |
| 10760877 | 4.85E−11 | ENSRNOG00000039214 |
| 10934637 | 4.87E−11 | ENSRNOG00000037838 |
| 10706705 | 4.91E−11 | ENSRNOG00000037247 |
| 10768814 | 5.00E−11 | ENSRNOG00000042188 |
| 10820548 | 5.14E−11 | ENSRNOG00000029304 |
| 10915648 | 5.18E−11 | ENSRNOG00000011582 |
| 10934071 | 5.23E−11 | ENSRNOG00000035505 |
| 10838497 | 5.24E−11 | ENSRNOG00000005814 |
| 10769897 | 5.32E−11 | ENSRNOG00000025069 |
| 10861140 | 5.38E−11 | ENSRNOG00000006066 |
| 10700681 | 5.39E−11 | ENSRNOG00000018091 |
| 10924672 | 5.40E−11 | ENSRNOG00000014718 |
| 10896042 | 5.56E−11 | ENSRNOG00000006870 |
| 10923595 | 5.60E−11 | ENSRNOG00000012331 |
| 10756129 | 5.77E−11 | ENSRNOG00000029881 |
| 10752007 | 5.79E−11 | ENSRNOG00000001796 |
| 10879446 | 5.88E−11 | ENSRNOG00000037432 |
| 10909407 | 5.97E−11 | ENSRNOG00000006604 |
| 10706586 | 6.01E−11 | ENSRNOG00000019854 |
| 10820586 | 6.16E−11 | ENSRNOG00000025411 |
| 10900721 | 6.24E−11 | ENSRNOG00000026435 |
| 10904948 | 6.26E−11 | ENSRNOG00000028711 |
| 10796198 | 6.27E−11 | ENSRNOG00000019223 |
| 10894549 | 6.31E−11 | ENSRNOG00000008016 |
| 10866252 | 6.39E−11 | ENSRNOG00000031515 |
| 10880107 | 6.42E−11 | NA |
| 10776239 | 6.49E−11 | ENSRNOG00000002014 |
| 10745670 | 6.77E−11 | ENSRNOG00000030021 |
| 10755088 | 6.77E−11 | ENSRNOG00000028895 |
| 10922857 | 6.83E−11 | ENSRNOG00000014835 |
| 10788483 | 6.86E−11 | ENSRNOG00000011349 |
| 10720215 | 6.88E−11 | ENSRNOG00000019673 |
| 10884996 | 6.94E−11 | ENSRNOG00000026855 |
| 10823591 | 7.03E−11 | ENSRNOG00000010515 |
| 10784454 | 7.09E−11 | ENSRNOG00000021314 |
| 10723850 | 7.14E−11 | ENSRNOG00000016206 |
| 10743964 | 7.23E−11 | ENSRNOG00000037615 |
| 10899387 | 7.25E−11 | ENSRNOG00000007607 |
| 10837138 | 7.28E−11 | ENSRNOG00000004861 |
| 10791358 | 7.55E−11 | ENSRNOG00000014541 |
| 10771287 | 7.67E−11 | ENSRNOG00000002099 |
| 10825464 | 7.67E−11 | ENSRNOG00000015821 |
| 10758156 | 7.72E−11 | ENSRNOG00000001004 |
| 10765994 | 7.73E−11 | ENSRNOG00000021113 |
| 10889560 | 7.84E−11 | ENSRNOG00000007884 |
| 10788824 | 7.92E−11 | ENSRNOG00000015501 |
| 10778327 | 8.25E−11 | ENSRNOG00000007113 |
| 10886629 | 8.27E−11 | ENSRNOG00000015457 |
| 10798175 | 8.32E−11 | ENSRNOG00000018108 |
| 10787197 | 8.37E−11 | ENSRNOG00000015751 |
| 10905560 | 8.56E−11 | ENSRNOG00000018803 |
| 10901115 | 8.59E−11 | ENSRNOG00000007076 |
| 10917617 | 8.59E−11 | ENSRNOG00000015149 |
| 10781426 | 8.62E−11 | ENSRNOG00000015054 |
| 10916946 | 8.72E−11 | ENSRNOG00000015945 |
| 10892643 | 8.77E−11 | ENSRNOG00000030235 |
| 10854645 | 8.91E−11 | ENSRNOG00000007918 |
| 10800783 | 8.96E−11 | ENSRNOG00000027355 |
| 10894167 | 9.13E−11 | ENSRNOG00000029478 |
| 10900592 | 9.20E−11 | ENSRNOG00000015434 |
| 10896836 | 9.28E−11 | ENSRNOG00000025528 |
| 10856274 | 9.35E−11 | ENSRNOG00000007178 |
| 10927612 | 9.50E−11 | ENSRNOG00000013583 |
| 10895140 | 9.53E−11 | NA |
| 10767373 | 9.58E−11 | ENSRNOG00000003866 |
| 10868000 | 9.99E−11 | ENSRNOG00000006170 |
| 10831551 | 1.02E−10 | ENSRNOG00000030431 |
| 10866012 | 1.02E−10 | ENSRNOG00000008259 |
| 10753075 | 1.09E−10 | ENSRNOG00000002051 |
| 10823057 | 1.09E−10 | ENSRNOG00000010210 |
| 10734975 | 1.10E−10 | ENSRNOG00000010756 |
| 10725245 | 1.12E−10 | ENSRNOG00000017012 |
| 10739958 | 1.12E−10 | ENSRNOG00000029658 |
| 10847562 | 1.13E−10 | ENSRNOG00000008758 |
| 10801734 | 1.13E−10 | ENSRNOG00000017832 |
| 10877130 | 1.13E−10 | ENSRNOG00000015072 |
| 10799087 | 1.14E−10 | ENSRNOG00000021686 |
| 10777832 | 1.15E−10 | ENSRNOG00000018144 |
| 10896966 | 1.17E−10 | ENSRNOG00000010794 |
| 10907689 | 1.17E−10 | ENSRNOG00000036832 |
| 10852600 | 1.19E−10 | ENSRNOG00000016547 |
| 10763547 | 1.20E−10 | ENSRNOG00000025543 |
| 10741851 | 1.20E−10 | ENSRNOG00000003508 |
| 10751586 | 1.21E−10 | ENSRNOG00000001797 |
| 10844916 | 1.22E−10 | ENSRNOG00000025278 |
| 10867799 | 1.23E−10 | ENSRNOG00000025145 |
| 10895581 | 1.24E−10 | ENSRNOG00000004813 |
| 10857473 | 1.24E−10 | ENSRNOG00000024647 |
| 10795679 | 1.26E−10 | ENSRNOG00000002480 |
| 10705539 | 1.26E−10 | ENSRNOG00000019838 |
| 10919283 | 1.27E−10 | ENSRNOG00000014543 |
| 10822386 | 1.29E−10 | ENSRNOG00000012095 |
| 10735455 | 1.31E−10 | ENSRNOG00000015236 |
| 10798952 | 1.32E−10 | ENSRNOG00000018123 |
| 10818708 | 1.32E−10 | ENSRNOG00000011800 |
| 10938654 | 1.34E−10 | ENSRNOG00000003954 |
| 10784004 | 1.35E−10 | ENSRNOG00000033089 |
| 10717011 | 1.37E−10 | ENSRNOG00000040137 |
| 10796111 | 1.37E−10 | ENSRNOG00000018549 |
| 10821250 | 1.38E−10 | ENSRNOG00000026989 |
| 10939570 | 1.39E−10 | ENSRNOG00000004581 |
| 10738130 | 1.39E−10 | ENSRNOG00000009972 |
| 10819852 | 1.40E−10 | ENSRNOG00000036823 |
| 10802274 | 1.42E−10 | ENSRNOG00000017181 |
| 10880095 | 1.42E−10 | ENSRNOG00000012989 |
| 10910204 | 1.42E−10 | ENSRNOG00000016413 |
| 10851407 | 1.46E−10 | ENSRNOG00000016744 |
| 10856232 | 1.49E−10 | ENSRNOG00000006069 |
| 10791144 | 1.50E−10 | ENSRNOG00000027257 |
| 10828021 | 1.50E−10 | ENSRNOG00000000837 |
| 10731444 | 1.50E−10 | ENSRNOG00000032439 |
| 10907198 | 1.51E−10 | ENSRNOG00000019380 |
| 10892509 | 1.52E−10 | ENSRNOG00000030194 |
| 10895600 | 1.59E−10 | ENSRNOG00000007286 |
| 10865527 | 1.64E−10 | ENSRNOG00000015290 |
| 10701880 | 1.64E−10 | ENSRNOG00000012476 |
| 10779673 | 1.64E−10 | ENSRNOG00000010645 |
| 10743593 | 1.67E−10 | ENSRNOG00000027286 |
| 10725326 | 1.74E−10 | ENSRNOG00000014946 |
| 10810378 | 1.74E−10 | ENSRNOG00000018382 |
| 10821851 | 1.77E−10 | ENSRNOG00000017413 |
| 10840673 | 1.78E−10 | ENSRNOG00000006767 |
| 10786115 | 1.79E−10 | ENSRNOG00000031445 |
| 10780094 | 1.79E−10 | NA |
| 10924853 | 1.82E−10 | ENSRNOG00000017359 |
| 10843959 | 1.83E−10 | ENSRNOG00000007422 |
| 10716080 | 1.85E−10 | ENSRNOG00000014061 |
| 10888125 | 1.86E−10 | ENSRNOG00000034127 |
| 10799558 | 1.87E−10 | ENSRNOG00000018044 |
| 10940673 | 1.90E−10 | NA |
| 10739915 | 1.97E−10 | ENSRNOG00000002949 |
| 10773754 | 2.00E−10 | ENSRNOG00000024945 |
| 10798021 | 2.01E−10 | ENSRNOG00000015290 |
| 10729913 | 2.08E−10 | ENSRNOG00000016117 |
| 10858315 | 2.09E−10 | ENSRNOG00000011153 |
| 10856053 | 2.11E−10 | ENSRNOG00000005890 |
| 10781616 | 2.13E−10 | ENSRNOG00000009471 |
| 10767465 | 2.13E−10 | ENSRNOG00000004470 |
| 10845647 | 2.14E−10 | ENSRNOG00000030763 |
| 10780433 | 2.14E−10 | ENSRNOG00000019478 |
| 10804384 | 2.16E−10 | ENSRNOG00000003578 |
| 10798635 | 2.21E−10 | ENSRNOG00000012418 |
| 10846158 | 2.22E−10 | ENSRNOG00000019047 |
| 10910071 | 2.23E−10 | ENSRNOG00000010277 |
| 10940660 | 2.23E−10 | ENSRNOG00000043069 |
| 10871173 | 2.24E−10 | ENSRNOG00000012020 |
| 10704956 | 2.26E−10 | ENSRNOG00000037931 |
| 10815763 | 2.29E−10 | ENSRNOG00000011238 |
| 10714763 | 2.35E−10 | ENSRNOG00000011308 |
| 10939018 | 2.35E−10 | ENSRNOG00000002291 |
| 10763284 | 2.36E−10 | ENSRNOG00000002882 |
| 10781733 | 2.39E−10 | ENSRNOG00000011657 |
| 10706650 | 2.41E−10 | ENSRNOG00000020318 |
| 10775283 | 2.41E−10 | ENSRNOG00000022032 |
| 10892109 | 2.42E−10 | ENSRNOG00000005776 |
| 10733750 | 2.44E−10 | ENSRNOG00000031544 |
| 10929321 | 2.45E−10 | ENSRNOG00000016165 |
| 10812162 | 2.45E−10 | ENSRNOG00000009997 |
| 10856089 | 2.52E−10 | ENSRNOG00000038379 |
| 10897465 | 2.55E−10 | ENSRNOG00000007679 |
| 10793748 | 2.56E−10 | NA |
| 10757808 | 2.56E−10 | NA |
| 10852799 | 2.59E−10 | ENSRNOG00000009085 |
| 10765186 | 2.63E−10 | ENSRNOG00000002776 |
| 10728912 | 2.66E−10 | ENSRNOG00000020980 |
| 10707832 | 2.67E−10 | ENSRNOG00000012698 |
| 10862818 | 2.67E−10 | ENSRNOG00000023657 |
| 10884868 | 2.70E−10 | ENSRNOG00000023098 |
| 10840183 | 2.70E−10 | ENSRNOG00000033119 |
| 10778763 | 2.75E−10 | ENSRNOG00000006205 |
| 10828026 | 2.76E−10 | ENSRNOG00000000855 |
| 10768560 | 2.79E−10 | ENSRNOG00000002454 |
| 10809392 | 2.80E−10 | ENSRNOG00000025764 |
| 10924667 | 2.81E−10 | ENSRNOG00000025970 |
| 10718591 | 2.82E−10 | ENSRNOG00000016588 |
| 10723196 | 2.89E−10 | ENSRNOG00000011975 |
| 10830230 | 2.89E−10 | ENSRNOG00000000815 |
| 10930259 | 2.90E−10 | ENSRNOG00000042189 |
| 10778708 | 2.99E−10 | ENSRNOG00000009267 |
| 10827292 | 3.04E−10 | ENSRNOG00000016262 |
| 10903400 | 3.06E−10 | ENSRNOG00000006816 |
| 10712657 | 3.10E−10 | ENSRNOG00000014254 |
| 10826335 | 3.11E−10 | ENSRNOG00000016214 |
| 10916187 | 3.13E−10 | ENSRNOG00000032180 |
| 10908482 | 3.15E−10 | ENSRNOG00000009271 |
| 10828884 | 3.16E−10 | ENSRNOG00000000529 |
| 10776978 | 3.19E−10 | ENSRNOG00000012367 |
| 10700252 | 3.24E−10 | NA |
| 10887657 | 3.25E−10 | ENSRNOG00000030194 |
| 10779832 | 3.29E−10 | ENSRNOG00000025625 |
| 10940569 | 3.49E−10 | NA |
| 10842704 | 3.55E−10 | ENSRNOG00000026887 |
| 10847745 | 3.55E−10 | ENSRNOG00000005136 |
| 10801980 | 3.62E−10 | ENSRNOG00000038955 |
| 10827349 | 3.62E−10 | ENSRNOG00000022218 |
| 10705485 | 3.64E−10 | ENSRNOG00000019229 |
| 10824530 | 3.69E−10 | ENSRNOG00000020811 |
| 10784307 | 3.72E−10 | ENSRNOG00000012644 |
| 10715962 | 3.74E−10 | ENSRNOG00000011339 |
| 10781927 | 3.78E−10 | ENSRNOG00000024125 |
| 10889263 | 3.79E−10 | ENSRNOG00000004110 |
| 10886345 | 3.80E−10 | NA |
| 10748870 | 3.81E−10 | ENSRNOG00000021424 |
| 10925127 | 3.81E−10 | ENSRNOG00000023238 |
| 10849290 | 3.83E−10 | ENSRNOG00000000168 |
| 10750505 | 3.83E−10 | ENSRNOG00000001628 |
| 10773973 | 3.84E−10 | ENSRNOG00000011940 |
| 10825472 | 3.86E−10 | ENSRNOG00000030019 |
| 10903246 | 3.90E−10 | ENSRNOG00000011278 |
| 10906608 | 3.90E−10 | ENSRNOG00000006305 |
| 10759846 | 3.91E−10 | ENSRNOG00000030410 |
| 10921951 | 3.93E−10 | ENSRNOG00000011987 |
| 10902506 | 3.96E−10 | ENSRNOG00000021317 |
| 10894221 | 4.00E−10 | ENSRNOG00000004572 |
| 10919453 | 4.04E−10 | ENSRNOG00000016384 |
| 10913592 | 4.10E−10 | ENSRNOG00000020804 |
| 10754785 | 4.11E−10 | ENSRNOG00000001766 |
| 10936660 | 4.17E−10 | ENSRNOG00000025358 |
| 10887622 | 4.25E−10 | ENSRNOG00000027990 |
| 10785897 | 4.29E−10 | ENSRNOG00000026229 |
| 10844331 | 4.33E−10 | ENSRNOG00000013973 |
| 10834109 | 4.35E−10 | ENSRNOG00000005871 |
| 10870733 | 4.38E−10 | ENSRNOG00000012778 |
| 10831640 | 4.41E−10 | ENSRNOG00000026669 |
| 10796418 | 4.41E−10 | ENSRNOG00000016403 |
| 10771669 | 4.43E−10 | ENSRNOG00000022229 |
| 10700792 | 4.50E−10 | ENSRNOG00000013741 |
| 10909321 | 4.52E−10 | ENSRNOG00000006312 |
| 10783203 | 4.52E−10 | ENSRNOG00000032133 |
| 10787841 | 4.54E−10 | ENSRNOG00000032297 |
| 10906169 | 4.71E−10 | ENSRNOG00000007133 |
| 10725235 | 4.79E−10 | ENSRNOG00000017136 |
| 10833588 | 4.80E−10 | ENSRNOG00000000579 |
| 10783964 | 4.81E−10 | ENSRNOG00000020465 |
| 10910158 | 4.86E−10 | ENSRNOG00000022702 |
| 10747494 | 4.88E−10 | ENSRNOG00000019075 |
| 10839307 | 4.90E−10 | ENSRNOG00000029643 |
| 10891910 | 4.95E−10 | ENSRNOG00000026605 |
| 10892478 | 4.99E−10 | ENSRNOG00000037528 |
| 10889360 | 5.04E−10 | ENSRNOG00000008104 |
| 10916955 | 5.12E−10 | ENSRNOG00000016069 |
| 10708565 | 5.15E−10 | ENSRNOG00000018322 |
| 10762044 | 5.26E−10 | ENSRNOG00000021440 |
| 10739988 | 5.28E−10 | ENSRNOG00000029658 |
| 10923031 | 5.33E−10 | ENSRNOG00000022762 |
| 10822099 | 5.38E−10 | ENSRNOG00000012017 |
| 10738576 | 5.39E−10 | ENSRNOG00000021031 |
| 10773198 | 5.39E−10 | ENSRNOG00000028534 |
| 10707912 | 5.46E−10 | ENSRNOG00000022822 |
| 10710795 | 5.46E−10 | ENSRNOG00000015773 |
| 10763073 | 5.50E−10 | ENSRNOG00000037441 |
| 10724389 | 5.61E−10 | ENSRNOG00000036804 |
| 10923700 | 5.61E−10 | ENSRNOG00000022066 |
| 10766304 | 5.69E−10 | ENSRNOG00000003087 |
| 10832705 | 5.71E−10 | ENSRNOG00000024019 |
| 10904812 | 5.77E−10 | ENSRNOG00000023781 |
| 10789774 | 5.78E−10 | ENSRNOG00000013264 |
| 10925324 | 5.82E−10 | ENSRNOG00000019892 |
| 10894942 | 5.98E−10 | ENSRNOG00000005637 |
| 10850390 | 6.00E−10 | ENSRNOG00000006077 |
| 10917870 | 6.03E−10 | ENSRNOG00000008400 |
| 10791579 | 6.04E−10 | ENSRNOG00000000108 |
| 10862410 | 6.10E−10 | ENSRNOG00000033338 |
| 10754088 | 6.24E−10 | ENSRNOG00000043100 |
| 10912456 | 6.26E−10 | ENSRNOG00000030463 |
| 10876295 | 6.32E−10 | ENSRNOG00000017485 |
| 10797019 | 6.37E−10 | ENSRNOG00000029294 |
| 10733243 | 6.39E−10 | ENSRNOG00000003399 |
| 10863269 | 6.43E−10 | ENSRNOG00000014617 |
| 10707455 | 6.48E−10 | ENSRNOG00000013718 |
| 10750144 | 6.49E−10 | ENSRNOG00000021569 |
| 10883858 | 6.54E−10 | ENSRNOG00000007379 |
| 10921168 | 6.64E−10 | NA |
| 10833005 | 6.66E−10 | ENSRNOG00000000568 |
| 10755148 | 6.70E−10 | ENSRNOG00000001806 |
| 10885417 | 6.75E−10 | ENSRNOG00000006570 |
| 10889607 | 6.76E−10 | ENSRNOG00000009385 |
| 10932534 | 6.81E−10 | ENSRNOG00000039496 |
| 10793002 | 6.83E−10 | ENSRNOG00000016483 |
| 10769547 | 6.83E−10 | ENSRNOG00000003283 |
| 10802995 | 7.02E−10 | ENSRNOG00000016258 |
| 10749874 | 7.17E−10 | ENSRNOG00000031531 |
| 10940659 | 7.20E−10 | ENSRNOG00000043391 |
| 10813007 | 7.23E−10 | ENSRNOG00000013963 |
| 10873203 | 7.29E−10 | ENSRNOG00000014241 |
| 10767268 | 7.30E−10 | ENSRNOG00000003769 |
| 10916000 | 7.33E−10 | ENSRNOG00000008904 |
| 10723928 | 7.45E−10 | ENSRNOG00000018164 |
| 10717170 | 7.45E−10 | ENSRNOG00000012346 |
| 10728936 | 7.57E−10 | ENSRNOG00000020997 |
| 10746547 | 7.61E−10 | ENSRNOG00000005575 |
| 10731428 | 7.66E−10 | ENSRNOG00000002335 |
| 10803377 | 7.67E−10 | ENSRNOG00000017031 |
| 10775484 | 7.68E−10 | ENSRNOG00000002095 |
| 10796347 | 7.72E−10 | ENSRNOG00000017981 |
| 10875375 | 7.74E−10 | ENSRNOG00000005669 |
| 10745931 | 7.75E−10 | ENSRNOG00000003967 |
| 10738556 | 7.84E−10 | ENSRNOG00000020916 |
| 10912640 | 7.90E−10 | ENSRNOG00000009789 |
| 10749172 | 7.98E−10 | ENSRNOG00000008755 |
| 10755094 | 8.02E−10 | ENSRNOG00000001823 |
| 10871444 | 8.03E−10 | ENSRNOG00000019484 |
| 10769788 | 8.17E−10 | ENSRNOG00000003138 |
| 10707237 | 8.18E−10 | ENSRNOG00000014829 |
| 10925267 | 8.24E−10 | ENSRNOG00000019476 |
| 10911593 | 8.49E−10 | ENSRNOG00000022968 |
| 10733531 | 8.67E−10 | ENSRNOG00000007515 |
| 10822864 | 8.70E−10 | ENSRNOG00000015816 |
| 10792163 | 8.72E−10 | ENSRNOG00000012582 |
| 10887647 | 8.77E−10 | ENSRNOG00000030194 |
| 10773496 | 8.83E−10 | ENSRNOG00000013805 |
| 10808223 | 8.93E−10 | ENSRNOG00000012027 |
| 10716562 | 8.96E−10 | ENSRNOG00000023690 |
| 10702746 | 8.99E−10 | ENSRNOG00000016919 |
| 10937499 | 9.06E−10 | ENSRNOG00000003180 |
| 10866788 | 9.10E−10 | ENSRNOG00000014382 |
| 10898862 | 9.24E−10 | ENSRNOG00000005965 |
| 10870987 | 9.26E−10 | ENSRNOG00000008025 |
| 10920745 | 9.34E−10 | ENSRNOG00000013265 |
| 10862586 | 9.41E−10 | ENSRNOG00000008063 |
| 10832833 | 9.45E−10 | ENSRNOG00000000648 |
| 10819512 | 9.46E−10 | ENSRNOG00000028768 |
| 10874298 | 9.64E−10 | ENSRNOG00000010580 |
| 10928668 | 9.82E−10 | ENSRNOG00000027430 |
| 10736330 | 9.83E−10 | NA |
| 10837170 | 1.00E−09 | NA |
| 10718504 | 1.02E−09 | ENSRNOG00000030642 |
| 10765248 | 1.04E−09 | ENSRNOG00000002839 |
| 10887774 | 1.04E−09 | ENSRNOG00000005271 |
| 10766521 | 1.06E−09 | ENSRNOG00000002393 |
| 10853471 | 1.06E−09 | ENSRNOG00000007234 |
| 10755186 | 1.07E−09 | ENSRNOG00000001773 |
| 10862473 | 1.08E−09 | ENSRNOG00000010011 |
| 10731514 | 1.08E−09 | ENSRNOG00000002635 |
| 10777571 | 1.08E−09 | ENSRNOG00000011073 |
| 10871494 | 1.08E−09 | ENSRNOG00000007087 |
| 10774375 | 1.08E−09 | ENSRNOG00000006329 |
| 10712841 | 1.09E−09 | ENSRNOG00000021724 |
| 10914618 | 1.09E−09 | ENSRNOG00000006774 |
| 10932795 | 1.11E−09 | ENSRNOG00000019180 |
| 10832478 | 1.13E−09 | ENSRNOG00000028394 |
| 10797631 | 1.14E−09 | ENSRNOG00000014818 |
| 10883357 | 1.16E−09 | ENSRNOG00000026649 |
| 10915555 | 1.18E−09 | ENSRNOG00000020901 |
| 10874655 | 1.20E−09 | ENSRNOG00000016936 |
| 10887306 | 1.21E−09 | ENSRNOG00000010165 |
| 10796858 | 1.23E−09 | ENSRNOG00000017254 |
| 10762115 | 1.24E−09 | ENSRNOG00000001350 |
| 10701325 | 1.26E−09 | NA |
| 10736802 | 1.27E−09 | NA |
| 10831957 | 1.28E−09 | ENSRNOG00000000511 |
| 10937805 | 1.32E−09 | ENSRNOG00000005157 |
| 10724073 | 1.32E−09 | ENSRNOG00000019687 |
| 10936248 | 1.33E−09 | ENSRNOG00000007059 |
| 10887270 | 1.33E−09 | ENSRNOG00000008145 |
| 10754367 | 1.34E−09 | ENSRNOG00000033933 |
| 10787784 | 1.38E−09 | ENSRNOG00000029400 |
| 10875503 | 1.41E−09 | ENSRNOG00000014664 |
| 10734475 | 1.41E−09 | ENSRNOG00000003367 |
| 10927842 | 1.43E−09 | ENSRNOG00000014079 |
| 10852115 | 1.45E−09 | ENSRNOG00000009530 |
| 10812407 | 1.46E−09 | ENSRNOG00000029210 |
| 10815308 | 1.50E−09 | ENSRNOG00000013296 |
| 10791233 | 1.50E−09 | ENSRNOG00000011891 |
| 10843793 | 1.52E−09 | ENSRNOG00000019322 |
| 10779028 | 1.53E−09 | NA |
| 10779811 | 1.53E−09 | ENSRNOG00000009663 |
| 10838192 | 1.54E−09 | ENSRNOG00000009401 |
| 10724987 | 1.54E−09 | ENSRNOG00000014318 |
| 10707418 | 1.55E−09 | ENSRNOG00000011945 |
| 10702897 | 1.55E−09 | ENSRNOG00000018321 |
| 10825696 | 1.58E−09 | ENSRNOG00000014385 |
| 10730519 | 1.63E−09 | ENSRNOG00000018468 |
| 10733067 | 1.64E−09 | ENSRNOG00000002499 |
| 10777492 | 1.68E−09 | ENSRNOG00000008474 |
| 10802434 | 1.70E−09 | ENSRNOG00000017729 |
| 10824680 | 1.70E−09 | ENSRNOG00000013356 |
| 10770795 | 1.71E−09 | ENSRNOG00000005861 |
| 10847957 | 1.71E−09 | ENSRNOG00000022710 |
| 10714136 | 1.71E−09 | ENSRNOG00000012611 |
| 10872665 | 1.71E−09 | ENSRNOG00000012965 |
| 10860951 | 1.72E−09 | ENSRNOG00000007546 |
| 10855393 | 1.73E−09 | ENSRNOG00000038740 |
| 10818108 | 1.74E−09 | ENSRNOG00000012630 |
| 10921274 | 1.79E−09 | ENSRNOG00000012942 |
| 10833394 | 1.79E−09 | ENSRNOG00000000800 |
| 10903459 | 1.80E−09 | ENSRNOG00000006118 |
| 10771426 | 1.81E−09 | ENSRNOG00000002251 |
| 10864550 | 1.87E−09 | ENSRNOG00000037397 |
| 10767132 | 1.87E−09 | ENSRNOG00000002625 |
| 10735827 | 1.93E−09 | ENSRNOG00000002980 |
| 10815425 | 1.94E−09 | ENSRNOG00000013485 |
| 10857667 | 1.95E−09 | ENSRNOG00000006941 |
| 10724637 | 1.96E−09 | ENSRNOG00000010186 |
| 10748061 | 1.96E−09 | ENSRNOG00000028521 |
| 10744283 | 2.01E−09 | ENSRNOG00000015674 |
| 10830561 | 2.01E−09 | ENSRNOG00000000302 |
| 10842384 | 2.02E−09 | ENSRNOG00000007665 |
| 10740000 | 2.06E−09 | ENSRNOG00000029658 |
| 10860815 | 2.07E−09 | ENSRNOG00000039800 |
| 10785263 | 2.08E−09 | ENSRNOG00000009412 |
| 10744995 | 2.09E−09 | ENSRNOG00000006674 |
| 10727405 | 2.13E−09 | ENSRNOG00000017512 |
| 10790096 | 2.20E−09 | ENSRNOG00000018823 |
| 10868289 | 2.21E−09 | ENSRNOG00000007029 |
| 10733001 | 2.22E−09 | ENSRNOG00000021425 |
| 10939260 | 2.22E−09 | ENSRNOG00000026961 |
| 10734882 | 2.22E−09 | ENSRNOG00000007387 |
| 10913186 | 2.25E−09 | ENSRNOG00000031965 |
| 10759616 | 2.25E−09 | ENSRNOG00000000994 |
| 10772194 | 2.28E−09 | ENSRNOG00000024689 |
| 10738237 | 2.30E−09 | ENSRNOG00000036814 |
| 10895397 | 2.33E−09 | ENSRNOG00000003890 |
| 10825665 | 2.33E−09 | ENSRNOG00000012946 |
| 10877069 | 2.34E−09 | ENSRNOG00000013656 |
| 10742352 | 2.34E−09 | ENSRNOG00000032396 |
| 10811663 | 2.42E−09 | ENSRNOG00000014461 |
| 10746051 | 2.43E−09 | ENSRNOG00000006796 |
| 10911287 | 2.43E−09 | ENSRNOG00000010362 |
| 10771475 | 2.48E−09 | ENSRNOG00000002292 |
| 10913311 | 2.49E−09 | ENSRNOG00000020450 |
| 10905439 | 2.50E−09 | ENSRNOG00000013076 |
| 10855377 | 2.51E−09 | ENSRNOG00000020169 |
| 10808393 | 2.51E−09 | ENSRNOG00000017431 |
| 10785286 | 2.52E−09 | ENSRNOG00000038291 |
| 10935739 | 2.56E−09 | ENSRNOG00000023333 |
| 10861736 | 2.56E−09 | ENSRNOG00000009513 |
| 10759287 | 2.65E−09 | ENSRNOG00000037483 |
| 10740018 | 2.66E−09 | ENSRNOG00000029658 |
| 10717303 | 2.67E−09 | ENSRNOG00000039865 |
| 10866374 | 2.72E−09 | ENSRNOG00000006338 |
| 10798142 | 2.73E−09 | ENSRNOG00000016420 |
| 10807583 | 2.78E−09 | ENSRNOG00000020333 |
| 10724580 | 2.79E−09 | ENSRNOG00000019521 |
| 10871133 | 2.83E−09 | ENSRNOG00000010381 |
| 10796965 | 2.84E−09 | ENSRNOG00000019175 |
| 10912161 | 2.85E−09 | ENSRNOG00000014064 |
| 10759769 | 2.87E−09 | ENSRNOG00000001098 |
| 10782271 | 2.90E−09 | ENSRNOG00000010516 |
| 10728960 | 2.91E−09 | ENSRNOG00000021013 |
| 10718001 | 2.91E−09 | ENSRNOG00000017731 |
| 10710154 | 2.97E−09 | ENSRNOG00000018459 |
| 10799291 | 3.00E−09 | ENSRNOG00000016885 |
| 10885560 | 3.01E−09 | ENSRNOG00000011139 |
| 10780396 | 3.14E−09 | ENSRNOG00000019041 |
| 10816834 | 3.14E−09 | ENSRNOG00000020744 |
| 10845407 | 3.15E−09 | ENSRNOG00000004772 |
| 10939837 | 3.16E−09 | ENSRNOG00000000881 |
| 10920056 | 3.19E−09 | ENSRNOG00000014832 |
| 10739992 | 3.26E−09 | ENSRNOG00000029658 |
| 10728924 | 3.27E−09 | ENSRNOG00000030689 |
| 10784117 | 3.27E−09 | ENSRNOG00000008855 |
| 10858370 | 3.28E−09 | ENSRNOG00000037198 |
| 10876479 | 3.34E−09 | ENSRNOG00000013956 |
| 10739998 | 3.35E−09 | ENSRNOG00000029658 |
| 10839197 | 3.36E−09 | ENSRNOG00000016357 |
| 10783253 | 3.36E−09 | ENSRNOG00000011953 |
| 10797189 | 3.38E−09 | ENSRNOG00000021637 |
| 10838170 | 3.46E−09 | ENSRNOG00000008510 |
| 10936482 | 3.50E−09 | ENSRNOG00000010208 |
| 10913664 | 3.51E−09 | ENSRNOG00000024330 |
| 10890967 | 3.52E−09 | ENSRNOG00000006399 |
| 10813984 | 3.54E−09 | ENSRNOG00000011997 |
| 10703706 | 3.61E−09 | NA |
| 10871182 | 3.61E−09 | ENSRNOG00000011019 |
| 10931692 | 3.64E−09 | ENSRNOG00000040025 |
| 10916432 | 3.65E−09 | ENSRNOG00000007166 |
| 10852034 | 3.66E−09 | ENSRNOG00000012762 |
| 10901842 | 3.66E−09 | ENSRNOG00000004011 |
| 10732652 | 3.68E−09 | ENSRNOG00000003977 |
| 10715475 | 3.72E−09 | ENSRNOG00000012684 |
| 10879817 | 3.76E−09 | ENSRNOG00000027320 |
| 10710868 | 3.79E−09 | ENSRNOG00000017403 |
| 10899913 | 3.81E−09 | ENSRNOG00000022943 |
| 10784140 | 3.87E−09 | ENSRNOG00000010137 |
| 10781441 | 3.89E−09 | ENSRNOG00000015577 |
| 10902630 | 3.97E−09 | ENSRNOG00000007834 |
| 10813778 | 4.01E−09 | ENSRNOG00000013451 |
| 10887638 | 4.01E−09 | ENSRNOG00000027874 |
| 10901621 | 4.12E−09 | ENSRNOG00000005823 |
| 10749307 | 4.16E−09 | ENSRNOG00000011459 |
| 10780505 | 4.18E−09 | ENSRNOG00000020399 |
| 10903466 | 4.21E−09 | ENSRNOG00000005333 |
| 10852437 | 4.28E−09 | ENSRNOG00000011624 |
| 10702880 | 4.30E−09 | ENSRNOG00000018158 |
| 10841774 | 4.30E−09 | ENSRNOG00000015393 |
| 10940550 | 4.38E−09 | NA |
| 10801386 | 4.38E−09 | ENSRNOG00000018849 |
| 10729715 | 4.38E−09 | ENSRNOG00000012536 |
| 10719692 | 4.39E−09 | ENSRNOG00000022669 |
| 10929556 | 4.46E−09 | NA |
| 10754337 | 4.47E−09 | ENSRNOG00000038868 |
| 10754700 | 4.49E−09 | ENSRNOG00000001774 |
| 10816470 | 4.51E−09 | ENSRNOG00000031778 |
| 10879726 | 4.54E−09 | ENSRNOG00000009131 |
| 10742617 | 4.59E−09 | ENSRNOG00000033065 |
| 10880074 | 4.61E−09 | ENSRNOG00000013179 |
| 10892007 | 4.71E−09 | ENSRNOG00000010711 |
| 10731493 | 4.73E−09 | ENSRNOG00000002520 |
| 10927875 | 4.76E−09 | ENSRNOG00000014079 |
| 10937867 | 4.83E−09 | ENSRNOG00000038624 |
| 10915071 | 4.86E−09 | ENSRNOG00000010999 |
| 10866835 | 4.90E−09 | ENSRNOG00000027630 |
| 10806829 | 4.92E−09 | ENSRNOG00000004131 |
| 10804511 | 4.93E−09 | ENSRNOG00000023197 |
| 10923127 | 4.98E−09 | ENSRNOG00000003940 |
| 10730312 | 5.00E−09 | ENSRNOG00000014675 |
| 10715483 | 5.01E−09 | ENSRNOG00000012763 |
| 10844520 | 5.20E−09 | ENSRNOG00000017172 |
| 10759248 | 5.26E−09 | ENSRNOG00000037505 |
| 10887120 | 5.26E−09 | ENSRNOG00000004973 |
| 10824115 | 5.39E−09 | ENSRNOG00000016451 |
| 10840519 | 5.40E−09 | ENSRNOG00000011785 |
| 10780716 | 5.42E−09 | ENSRNOG00000032888 |
| 10861986 | 5.42E−09 | ENSRNOG00000006859 |
| 10808431 | 5.46E−09 | ENSRNOG00000017869 |
| 10866041 | 5.55E−09 | ENSRNOG00000009638 |
| 10712299 | 5.59E−09 | ENSRNOG00000018367 |
| 10940663 | 5.63E−09 | NA |
| 10888153 | 5.64E−09 | ENSRNOG00000025899 |
| 10794353 | 5.69E−09 | ENSRNOG00000016560 |
| 10705364 | 5.73E−09 | ENSRNOG00000024363 |
| 10892493 | 5.75E−09 | ENSRNOG00000030194 |
| 10940549 | 5.76E−09 | NA |
| 10841348 | 5.80E−09 | ENSRNOG00000018755 |
| 10712935 | 5.83E−09 | ENSRNOG00000019708 |
| 10882752 | 5.97E−09 | ENSRNOG00000015415 |
| 10742254 | 6.08E−09 | ENSRNOG00000006557 |
| 10815176 | 6.09E−09 | ENSRNOG00000014066 |
| 10709844 | 6.15E−09 | ENSRNOG00000010017 |
| 10788569 | 6.26E−09 | ENSRNOG00000013328 |
| 10701393 | 6.34E−09 | NA |
| 10902003 | 6.36E−09 | ENSRNOG00000009094 |
| 10701535 | 6.48E−09 | NA |
| 10923951 | 6.50E−09 | ENSRNOG00000015158 |
| 10771883 | 6.51E−09 | ENSRNOG00000003666 |
| 10930717 | 6.52E−09 | NA |
| 10831620 | 6.54E−09 | ENSRNOG00000027144 |
| 10922843 | 6.55E−09 | NA |
| 10862361 | 6.56E−09 | ENSRNOG00000006228 |
| 10901904 | 6.58E−09 | ENSRNOG00000004367 |
| 10902080 | 6.62E−09 | NA |
| 10748969 | 6.67E−09 | ENSRNOG00000003670 |
| 10757898 | 6.70E−09 | ENSRNOG00000001480 |
| 10717813 | 6.71E−09 | ENSRNOG00000018052 |
| 10829831 | 6.80E−09 | ENSRNOG00000000387 |
| 10876831 | 6.85E−09 | ENSRNOG00000032048 |
| 10717106 | 6.90E−09 | ENSRNOG00000014979 |
| 10752222 | 6.97E−09 | ENSRNOG00000022792 |
| 10925468 | 7.03E−09 | ENSRNOG00000024030 |
| 10742483 | 7.08E−09 | ENSRNOG00000003645 |
| 10713974 | 7.09E−09 | ENSRNOG00000021644 |
| 10742145 | 7.12E−09 | ENSRNOG00000003256 |
| 10716480 | 7.12E−09 | ENSRNOG00000011439 |
| 10868165 | 7.14E−09 | ENSRNOG00000024247 |
| 10779794 | 7.19E−09 | ENSRNOG00000008892 |
| 10808652 | 7.19E−09 | ENSRNOG00000016631 |
| 10885251 | 7.20E−09 | ENSRNOG00000008292 |
| 10751136 | 7.23E−09 | ENSRNOG00000002013 |
| 10706117 | 7.25E−09 | ENSRNOG00000021142 |
| 10794944 | 7.26E−09 | NA |
| 10742744 | 7.26E−09 | ENSRNOG00000021370 |
| 10726860 | 7.31E−09 | ENSRNOG00000019351 |
| 10777290 | 7.40E−09 | ENSRNOG00000005043 |
| 10860765 | 7.40E−09 | ENSRNOG00000009258 |
| 10834908 | 7.41E−09 | ENSRNOG00000013491 |
| 10865956 | 7.43E−09 | ENSRNOG00000032553 |
| 10801381 | 7.50E−09 | ENSRNOG00000044553 |
| 10862415 | 7.52E−09 | ENSRNOG00000008465 |
| 10800969 | 7.55E−09 | ENSRNOG00000032878 |
| 10742288 | 7.60E−09 | ENSRNOG00000006860 |
| 10861560 | 7.61E−09 | NA |
| 10836212 | 7.62E−09 | ENSRNOG00000004617 |
| 10853710 | 7.64E−09 | ENSRNOG00000008524 |
| 10867479 | 7.65E−09 | ENSRNOG00000040157 |
| 10807722 | 7.66E−09 | ENSRNOG00000021379 |
| 10801975 | 7.72E−09 | ENSRNOG00000019542 |
| 10788204 | 7.73E−09 | ENSRNOG00000011116 |
| 10937932 | 7.75E−09 | ENSRNOG00000025290 |
| 10797127 | 7.76E−09 | ENSRNOG00000022870 |
| 10821117 | 7.79E−09 | ENSRNOG00000011561 |
| 10875988 | 7.91E−09 | ENSRNOG00000006384 |
| 10791394 | 7.96E−09 | ENSRNOG00000014541 |
| 10914614 | 7.98E−09 | ENSRNOG00000006755 |
| 10918392 | 8.20E−09 | ENSRNOG00000018184 |
| 10712517 | 8.25E−09 | ENSRNOG00000020451 |
| 10703710 | 8.36E−09 | ENSRNOG00000038821 |
| 10717003 | 8.44E−09 | ENSRNOG00000040140 |
| 10706381 | 8.44E−09 | ENSRNOG00000018334 |
| 10772915 | 8.48E−09 | ENSRNOG00000004932 |
| 10750958 | 8.48E−09 | ENSRNOG00000029564 |
| 10815815 | 8.52E−09 | ENSRNOG00000012873 |
| 10918535 | 8.55E−09 | ENSRNOG00000014934 |
| 10747426 | 8.78E−09 | ENSRNOG00000018247 |
| 10865369 | 8.81E−09 | ENSRNOG00000009211 |
| 10834098 | 8.85E−09 | ENSRNOG00000005751 |
| 10802867 | 8.89E−09 | ENSRNOG00000016987 |
| 10895499 | 8.99E−09 | ENSRNOG00000004411 |
| 10700756 | 9.03E−09 | NA |
| 10739990 | 9.16E−09 | ENSRNOG00000029658 |
| 10796094 | 9.27E−09 | ENSRNOG00000018011 |
| 10721682 | 9.36E−09 | ENSRNOG00000035072 |
| 10701156 | 9.40E−09 | NA |
| 10775278 | 9.56E−09 | ENSRNOG00000002129 |
| 10786028 | 9.60E−09 | ENSRNOG00000025094 |
| 10809524 | 9.65E−09 | ENSRNOG00000016643 |
| 10940471 | 9.83E−09 | ENSRNOG00000038908 |
| 10753135 | 1.01E−08 | ENSRNOG00000002028 |
| 10902584 | 1.02E−08 | ENSRNOG00000006541 |
| 10930076 | 1.02E−08 | ENSRNOG00000018904 |
| 10828638 | 1.03E−08 | ENSRNOG00000000498 |
| 10801541 | 1.04E−08 | ENSRNOG00000031421 |
| 10890991 | 1.05E−08 | ENSRNOG00000007271 |
| 10902885 | 1.06E−08 | ENSRNOG00000025592 |
| 10700303 | 1.07E−08 | NA |
| 10837688 | 1.07E−08 | ENSRNOG00000007241 |
| 10908393 | 1.08E−08 | ENSRNOG00000022741 |
| 10906643 | 1.10E−08 | ENSRNOG00000007032 |
| 10700369 | 1.11E−08 | NA |
| 10761526 | 1.12E−08 | ENSRNOG00000000967 |
| 10903896 | 1.14E−08 | ENSRNOG00000006738 |
| 10773168 | 1.15E−08 | ENSRNOG00000013167 |
| 10728930 | 1.16E−08 | NA |
| 10722131 | 1.16E−08 | ENSRNOG00000012232 |
| 10917692 | 1.17E−08 | ENSRNOG00000016474 |
| 10703443 | 1.17E−08 | ENSRNOG00000031331 |
| 10709799 | 1.19E−08 | ENSRNOG00000010427 |
| 10880805 | 1.19E−08 | ENSRNOG00000013954 |
| 10913835 | 1.20E−08 | ENSRNOG00000021047 |
| 10874152 | 1.23E−08 | ENSRNOG00000008717 |
| 10836169 | 1.24E−08 | ENSRNOG00000006267 |
| 10922476 | 1.25E−08 | ENSRNOG00000014162 |
| 10770351 | 1.26E−08 | ENSRNOG00000003629 |
| 10729771 | 1.26E−08 | ENSRNOG00000019190 |
| 10740688 | 1.28E−08 | ENSRNOG00000003855 |
| 10797009 | 1.30E−08 | ENSRNOG00000033448 |
| 10769771 | 1.30E−08 | ENSRNOG00000003138 |
| 10940624 | 1.33E−08 | NA |
| 10849488 | 1.34E−08 | ENSRNOG00000031598 |
| 10767189 | 1.36E−08 | ENSRNOG00000025430 |
| 10796220 | 1.36E−08 | ENSRNOG00000019336 |
| 10886509 | 1.37E−08 | ENSRNOG00000008728 |
| 10929842 | 1.38E−08 | ENSRNOG00000020254 |
| 10704536 | 1.38E−08 | ENSRNOG00000016434 |
| 10700872 | 1.39E−08 | NA |
| 10842964 | 1.40E−08 | ENSRNOG00000012722 |
| 10862285 | 1.40E−08 | ENSRNOG00000029329 |
| 10911524 | 1.42E−08 | ENSRNOG00000007533 |
| 10744918 | 1.42E−08 | ENSRNOG00000003123 |
| 10894857 | 1.42E−08 | ENSRNOG00000004148 |
| 10882812 | 1.43E−08 | ENSRNOG00000015796 |
| 10750996 | 1.43E−08 | ENSRNOG00000023030 |
| 10790002 | 1.48E−08 | ENSRNOG00000028461 |
| 10825962 | 1.48E−08 | ENSRNOG00000019465 |
| 10782870 | 1.50E−08 | ENSRNOG00000009481 |
| 10851925 | 1.50E−08 | ENSRNOG00000008194 |
| 10917301 | 1.51E−08 | ENSRNOG00000043498 |
| 10891818 | 1.52E−08 | ENSRNOG00000005470 |
| 10809303 | 1.52E−08 | NA |
| 10746095 | 1.53E−08 | ENSRNOG00000003919 |
| 10774094 | 1.56E−08 | ENSRNOG00000031045 |
| 10725397 | 1.57E−08 | ENSRNOG00000017277 |
| 10746286 | 1.57E−08 | ENSRNOG00000002835 |
| 10936537 | 1.58E−08 | ENSRNOG00000016217 |
| 10862014 | 1.59E−08 | ENSRNOG00000008196 |
| 10825686 | 1.59E−08 | ENSRNOG00000013538 |
| 10913469 | 1.60E−08 | ENSRNOG00000020656 |
| 10912112 | 1.62E−08 | ENSRNOG00000013229 |
| 10700753 | 1.63E−08 | ENSRNOG00000003670 |
| 10913487 | 1.67E−08 | ENSRNOG00000020667 |
| 10779940 | 1.68E−08 | ENSRNOG00000034431 |
| 10863213 | 1.68E−08 | ENSRNOG00000038138 |
| 10823570 | 1.69E−08 | ENSRNOG00000009808 |
| 10918031 | 1.70E−08 | ENSRNOG00000014080 |
| 10769486 | 1.71E−08 | ENSRNOG00000035035 |
| 10920981 | 1.72E−08 | ENSRNOG00000018509 |
| 10739986 | 1.73E−08 | ENSRNOG00000029658 |
| 10882571 | 1.73E−08 | ENSRNOG00000004685 |
| 10745266 | 1.75E−08 | ENSRNOG00000022657 |
| 10862876 | 1.75E−08 | ENSRNOG00000007270 |
| 10728578 | 1.76E−08 | ENSRNOG00000020202 |
| 10731168 | 1.77E−08 | ENSRNOG00000010117 |
| 10814340 | 1.80E−08 | NA |
| 10831135 | 1.81E−08 | ENSRNOG00000000841 |
| 10881178 | 1.82E−08 | ENSRNOG00000012503 |
| 10714890 | 1.82E−08 | ENSRNOG00000019142 |
| 10719728 | 1.83E−08 | ENSRNOG00000020263 |
| 10872994 | 1.84E−08 | ENSRNOG00000011910 |
| 10876466 | 1.84E−08 | ENSRNOG00000014365 |
| 10821900 | 1.84E−08 | ENSRNOG00000017876 |
| 10724047 | 1.84E−08 | ENSRNOG00000033196 |
| 10864910 | 1.87E−08 | ENSRNOG00000014713 |
| 10915437 | 1.87E−08 | ENSRNOG00000039859 |
| 10805066 | 1.88E−08 | ENSRNOG00000016879 |
| 10883865 | 1.88E−08 | ENSRNOG00000004506 |
| 10721126 | 1.90E−08 | ENSRNOG00000021573 |
| 10913995 | 1.92E−08 | ENSRNOG00000010434 |
| 10930506 | 1.92E−08 | ENSRNOG00000002110 |
| 10800216 | 1.93E−08 | ENSRNOG00000012597 |
| 10874708 | 1.94E−08 | ENSRNOG00000018691 |
| 10790734 | 1.94E−08 | ENSRNOG00000016186 |
| 10907062 | 1.94E−08 | ENSRNOG00000033667 |
| 10810727 | 1.96E−08 | ENSRNOG00000019494 |
| 10859438 | 1.97E−08 | ENSRNOG00000008747 |
| 10798558 | 1.98E−08 | ENSRNOG00000027672 |
| 10930588 | 2.01E−08 | ENSRNOG00000031053 |
| 10765820 | 2.01E−08 | ENSRNOG00000003480 |
| 10851280 | 2.02E−08 | ENSRNOG00000006921 |
| 10908082 | 2.02E−08 | ENSRNOG00000009695 |
| 10700573 | 2.02E−08 | NA |
| 10707388 | 2.03E−08 | ENSRNOG00000011480 |
| 10820163 | 2.04E−08 | ENSRNOG00000016384 |
| 10794756 | 2.04E−08 | ENSRNOG00000016226 |
| 10924199 | 2.05E−08 | ENSRNOG00000016503 |
| 10733017 | 2.05E−08 | ENSRNOG00000007243 |
| 10887667 | 2.06E−08 | ENSRNOG00000004262 |
| 10806296 | 2.07E−08 | NA |
| 10701141 | 2.09E−08 | NA |
| 10786465 | 2.10E−08 | ENSRNOG00000016064 |
| 10754939 | 2.11E−08 | ENSRNOG00000001728 |
| 10840347 | 2.12E−08 | ENSRNOG00000005924 |
| 10860801 | 2.14E−08 | ENSRNOG00000039801 |
| 10923670 | 2.15E−08 | ENSRNOG00000016486 |
| 10935605 | 2.16E−08 | ENSRNOG00000000871 |
| 10936278 | 2.18E−08 | ENSRNOG00000030645 |
| 10827299 | 2.26E−08 | ENSRNOG00000016432 |
| 10821072 | 2.27E−08 | ENSRNOG00000010720 |
| 10714103 | 2.27E−08 | ENSRNOG00000021084 |
| 10708903 | 2.29E−08 | ENSRNOG00000015808 |
| 10845072 | 2.32E−08 | ENSRNOG00000004677 |
| 10921290 | 2.36E−08 | ENSRNOG00000010637 |
| 10855387 | 2.36E−08 | ENSRNOG00000008369 |
| 10859581 | 2.37E−08 | ENSRNOG00000013816 |
| 10779683 | 2.38E−08 | ENSRNOG00000012255 |
| 10866544 | 2.39E−08 | ENSRNOG00000007047 |
| 10909783 | 2.40E−08 | ENSRNOG00000007325 |
| 10754010 | 2.41E−08 | ENSRNOG00000013721 |
| 10709020 | 2.42E−08 | ENSRNOG00000017079 |
| 10826547 | 2.42E−08 | ENSRNOG00000015250 |
| 10802579 | 2.43E−08 | ENSRNOG00000015777 |
| 10853193 | 2.43E−08 | ENSRNOG00000013431 |
| 10850613 | 2.43E−08 | ENSRNOG00000005920 |
| 10908391 | 2.44E−08 | NA |
| 10737770 | 2.45E−08 | ENSRNOG00000035539 |
| 10868191 | 2.48E−08 | ENSRNOG00000014713 |
| 10877860 | 2.49E−08 | ENSRNOG00000011498 |
| 10866030 | 2.53E−08 | ENSRNOG00000008375 |
| 10729293 | 2.55E−08 | ENSRNOG00000017936 |
| 10821050 | 2.57E−08 | ENSRNOG00000018903 |
| 10837832 | 2.58E−08 | ENSRNOG00000013594 |
| 10778204 | 2.61E−08 | ENSRNOG00000010904 |
| 10772066 | 2.62E−08 | ENSRNOG00000002011 |
| 10869267 | 2.62E−08 | ENSRNOG00000016692 |
| 10754214 | 2.63E−08 | ENSRNOG00000027151 |
| 10721249 | 2.63E−08 | ENSRNOG00000032990 |
| 10739977 | 2.67E−08 | ENSRNOG00000029658 |
| 10937174 | 2.69E−08 | ENSRNOG00000028611 |
| 10794345 | 2.71E−08 | ENSRNOG00000016525 |
| 10886298 | 2.72E−08 | ENSRNOG00000004083 |
| 10734740 | 2.72E−08 | ENSRNOG00000003992 |
| 10834945 | 2.77E−08 | ENSRNOG00000014316 |
| 10880610 | 2.78E−08 | ENSRNOG00000018576 |
| 10885586 | 2.79E−08 | NA |
| 10749983 | 2.86E−08 | ENSRNOG00000001557 |
| 10812526 | 2.87E−08 | ENSRNOG00000011032 |
| 10723156 | 2.87E−08 | ENSRNOG00000012002 |
| 10701345 | 2.88E−08 | NA |
| 10841789 | 2.89E−08 | ENSRNOG00000015614 |
| 10700828 | 2.90E−08 | NA |
| 10720891 | 2.94E−08 | ENSRNOG00000021097 |
| 10773849 | 2.99E−08 | ENSRNOG00000024390 |
| 10728616 | 3.00E−08 | ENSRNOG00000020346 |
| 10830102 | 3.01E−08 | ENSRNOG00000030934 |
| 10865463 | 3.03E−08 | ENSRNOG00000014294 |
| 10777232 | 3.03E−08 | ENSRNOG00000003069 |
| 10890745 | 3.07E−08 | ENSRNOG00000008049 |
| 10740209 | 3.10E−08 | ENSRNOG00000036677 |
| 10919041 | 3.12E−08 | ENSRNOG00000010240 |
| 10717935 | 3.17E−08 | ENSRNOG00000019048 |
| 10724052 | 3.18E−08 | ENSRNOG00000019413 |
| 10747228 | 3.18E−08 | ENSRNOG00000013754 |
| 10894643 | 3.22E−08 | ENSRNOG00000026807 |
| 10871975 | 3.24E−08 | ENSRNOG00000008279 |
| 10921343 | 3.25E−08 | ENSRNOG00000011321 |
| 10750117 | 3.27E−08 | ENSRNOG00000002104 |
| 10912396 | 3.32E−08 | ENSRNOG00000010942 |
| 10940641 | 3.38E−08 | NA |
| 10840368 | 3.39E−08 | ENSRNOG00000007160 |
| 10819489 | 3.39E−08 | ENSRNOG00000032240 |
| 10854190 | 3.40E−08 | ENSRNOG00000007437 |
| 10712506 | 3.42E−08 | ENSRNOG00000026039 |
| 10890182 | 3.44E−08 | ENSRNOG00000004629 |
| 10809044 | 3.47E−08 | ENSRNOG00000013301 |
| 10919433 | 3.50E−08 | ENSRNOG00000013802 |
| 10758212 | 3.51E−08 | ENSRNOG00000001061 |
| 10707707 | 3.51E−08 | ENSRNOG00000016090 |
| 10759018 | 3.56E−08 | ENSRNOG00000021725 |
| 10837266 | 3.63E−08 | ENSRNOG00000005256 |
| 10830498 | 3.66E−08 | ENSRNOG00000026352 |
| 10815706 | 3.67E−08 | ENSRNOG00000037934 |
| 10725645 | 3.68E−08 | ENSRNOG00000017429 |
| 10763808 | 3.69E−08 | ENSRNOG00000006963 |
| 10750825 | 3.70E−08 | ENSRNOG00000001608 |
| 10853797 | 3.70E−08 | ENSRNOG00000032978 |
| 10770666 | 3.71E−08 | ENSRNOG00000003583 |
| 10916686 | 3.79E−08 | ENSRNOG00000008526 |
| 10786359 | 3.80E−08 | ENSRNOG00000014750 |
| 10922962 | 3.82E−08 | NA |
| 10914201 | 3.86E−08 | ENSRNOG00000030287 |
| 10919214 | 3.90E−08 | ENSRNOG00000000204 |
| 10703715 | 3.93E−08 | ENSRNOG00000038794 |
| 10812229 | 3.95E−08 | ENSRNOG00000040297 |
| 10777328 | 4.00E−08 | ENSRNOG00000005374 |
| 10805335 | 4.04E−08 | ENSRNOG00000016753 |
| 10760789 | 4.06E−08 | ENSRNOG00000001325 |
| 10895037 | 4.07E−08 | NA |
| 10762217 | 4.09E−08 | ENSRNOG00000015457 |
| 10907585 | 4.11E−08 | ENSRNOG00000013445 |
| 10711769 | 4.13E−08 | ENSRNOG00000018066 |
| 10758599 | 4.19E−08 | ENSRNOG00000028523 |
| 10727924 | 4.25E−08 | ENSRNOG00000020726 |
| 10783521 | 4.28E−08 | ENSRNOG00000039428 |
| 10746000 | 4.30E−08 | ENSRNOG00000005596 |
| 10776347 | 4.34E−08 | ENSRNOG00000002101 |
| 10736661 | 4.36E−08 | ENSRNOG00000005538 |
| 10709552 | 4.40E−08 | ENSRNOG00000030818 |
| 10932740 | 4.41E−08 | ENSRNOG00000036839 |
| 10871616 | 4.44E−08 | ENSRNOG00000009341 |
| 10700449 | 4.45E−08 | ENSRNOG00000013741 |
| 10928684 | 4.49E−08 | ENSRNOG00000014960 |
| 10763299 | 4.52E−08 | ENSRNOG00000002821 |
| 10867010 | 4.53E−08 | ENSRNOG00000036917 |
| 10901014 | 4.54E−08 | ENSRNOG00000006167 |
| 10915740 | 4.65E−08 | ENSRNOG00000030758 |
| 10940637 | 4.71E−08 | NA |
| 10899204 | 4.72E−08 | ENSRNOG00000032588 |
| 10804603 | 4.78E−08 | ENSRNOG00000026108 |
| 10760403 | 4.79E−08 | ENSRNOG00000001079 |
| 10776597 | 4.80E−08 | ENSRNOG00000025882 |
| 10868662 | 4.80E−08 | ENSRNOG00000013078 |
| 10830408 | 4.82E−08 | ENSRNOG00000000593 |
| 10894878 | 4.83E−08 | ENSRNOG00000004494 |
| 10854897 | 4.84E−08 | NA |
| 10924918 | 4.84E−08 | ENSRNOG00000025731 |
| 10767985 | 4.85E−08 | NA |
| 10715848 | 4.90E−08 | ENSRNOG00000020011 |
| 10809370 | 5.00E−08 | ENSRNOG00000018564 |
| 10752050 | 5.04E−08 | ENSRNOG00000001783 |
| 10907992 | 5.08E−08 | ENSRNOG00000005713 |
| 10891436 | 5.11E−08 | ENSRNOG00000004300 |
| 10798390 | 5.11E−08 | ENSRNOG00000018804 |
| 10713328 | 5.13E−08 | ENSRNOG00000021061 |
| 10766289 | 5.16E−08 | ENSRNOG00000003717 |
| 10885396 | 5.24E−08 | NA |
| 10928522 | 5.25E−08 | NA |
| 10770070 | 5.34E−08 | ENSRNOG00000003365 |
| 10800726 | 5.35E−08 | ENSRNOG00000014089 |
| 10940444 | 5.42E−08 | NA |
| 10830261 | 5.42E−08 | ENSRNOG00000037757 |
| 10702936 | 5.42E−08 | ENSRNOG00000019091 |
| 10924876 | 5.42E−08 | ENSRNOG00000017730 |
| 10736957 | 5.51E−08 | ENSRNOG00000034013 |
| 10776968 | 5.51E−08 | ENSRNOG00000002201 |
| 10892409 | 5.54E−08 | NA |
| 10839743 | 5.55E−08 | ENSRNOG00000018205 |
| 10807311 | 5.63E−08 | ENSRNOG00000024452 |
| 10938858 | 5.63E−08 | ENSRNOG00000002790 |
| 10869158 | 5.68E−08 | ENSRNOG00000032048 |
| 10783474 | 5.74E−08 | NA |
| 10814817 | 5.77E−08 | ENSRNOG00000011261 |
| 10796234 | 5.81E−08 | ENSRNOG00000023661 |
| 10758497 | 5.81E−08 | ENSRNOG00000001316 |
| 10856415 | 5.82E−08 | ENSRNOG00000013668 |
| 10838538 | 5.82E−08 | ENSRNOG00000031760 |
| 10905589 | 5.83E−08 | ENSRNOG00000019070 |
| 10872101 | 5.88E−08 | ENSRNOG00000013154 |
| 10701418 | 5.89E−08 | NA |
| 10932187 | 5.90E−08 | ENSRNOG00000024098 |
| 10768332 | 5.96E−08 | ENSRNOG00000003687 |
| 10756386 | 5.99E−08 | ENSRNOG00000000921 |
| 10839857 | 6.06E−08 | ENSRNOG00000007128 |
| 10763367 | 6.07E−08 | ENSRNOG00000002460 |
| 10935843 | 6.10E−08 | ENSRNOG00000036553 |
| 10755069 | 6.12E−08 | ENSRNOG00000031669 |
| 10852987 | 6.16E−08 | ENSRNOG00000010601 |
| 10754384 | 6.22E−08 | ENSRNOG00000038835 |
| 10880033 | 6.38E−08 | ENSRNOG00000009705 |
| 10750029 | 6.38E−08 | ENSRNOG00000001549 |
| 10708745 | 6.41E−08 | ENSRNOG00000011882 |
| 10892499 | 6.43E−08 | ENSRNOG00000030194 |
| 10859027 | 6.45E−08 | ENSRNOG00000025751 |
| 10865993 | 6.51E−08 | ENSRNOG00000007909 |
| 10873934 | 6.54E−08 | ENSRNOG00000009615 |
| 10771171 | 6.54E−08 | NA |
| 10816483 | 6.56E−08 | ENSRNOG00000037555 |
| 10755206 | 6.59E−08 | ENSRNOG00000001764 |
| 10899826 | 6.61E−08 | ENSRNOG00000004904 |
| 10928254 | 6.62E−08 | ENSRNOG00000012558 |
| 10854863 | 6.69E−08 | NA |
| 10798624 | 6.71E−08 | ENSRNOG00000027571 |
| 10841850 | 6.71E−08 | ENSRNOG00000016340 |
| 10752257 | 6.79E−08 | NA |
| 10700664 | 6.90E−08 | NA |
| 10705553 | 6.93E−08 | ENSRNOG00000019928 |
| 10809399 | 6.94E−08 | ENSRNOG00000043098 |
| 10772408 | 6.99E−08 | ENSRNOG00000002278 |
| 10719922 | 7.01E−08 | ENSRNOG00000020743 |
| 10761162 | 7.06E−08 | ENSRNOG00000001442 |
| 10751469 | 7.08E−08 | ENSRNOG00000023334 |
| 10836956 | 7.13E−08 | ENSRNOG00000008717 |
| 10833688 | 7.18E−08 | ENSRNOG00000000312 |
| 10827438 | 7.39E−08 | ENSRNOG00000009992 |
| 10764388 | 7.46E−08 | ENSRNOG00000011063 |
| 10857314 | 7.48E−08 | ENSRNOG00000009019 |
| 10772330 | 7.50E−08 | ENSRNOG00000002196 |
| 10862464 | 7.50E−08 | ENSRNOG00000009970 |
| 10739302 | 7.53E−08 | ENSRNOG00000003612 |
| 10923182 | 7.55E−08 | ENSRNOG00000012375 |
| 10925687 | 7.55E−08 | ENSRNOG00000019087 |
| 10703139 | 7.78E−08 | ENSRNOG00000013190 |
| 10822799 | 7.81E−08 | ENSRNOG00000013293 |
| 10721616 | 7.96E−08 | ENSRNOG00000025877 |
| 10717920 | 7.96E−08 | ENSRNOG00000018915 |
| 10713796 | 8.03E−08 | NA |
| 10825925 | 8.06E−08 | ENSRNOG00000029726 |
| 10841037 | 8.08E−08 | ENSRNOG00000023335 |
| 10849943 | 8.33E−08 | ENSRNOG00000021234 |
| 10783507 | 8.36E−08 | ENSRNOG00000039345 |
| 10924669 | 8.37E−08 | ENSRNOG00000025970 |
| 10717141 | 8.38E−08 | ENSRNOG00000011245 |
| 10874245 | 8.38E−08 | ENSRNOG00000010109 |
| 10844808 | 8.40E−08 | ENSRNOG00000010150 |
| 10800522 | 8.48E−08 | ENSRNOG00000015301 |
| 10806927 | 8.49E−08 | ENSRNOG00000017879 |
| 10936346 | 8.50E−08 | ENSRNOG00000013092 |
| 10773340 | 8.53E−08 | ENSRNOG00000006400 |
| 10898335 | 8.54E−08 | ENSRNOG00000014637 |
| 10830654 | 8.93E−08 | ENSRNOG00000000325 |
| 10935831 | 8.98E−08 | ENSRNOG00000016884 |
| 10821450 | 9.10E−08 | ENSRNOG00000012080 |
| 10856367 | 9.21E−08 | ENSRNOG00000010761 |
| 10725519 | 9.27E−08 | ENSRNOG00000013836 |
| 10716646 | 9.28E−08 | ENSRNOG00000015239 |
| 10854558 | 9.31E−08 | ENSRNOG00000013251 |
| 10787889 | 9.36E−08 | ENSRNOG00000014541 |
| 10798135 | 9.38E−08 | ENSRNOG00000033772 |
| 10822377 | 9.53E−08 | ENSRNOG00000022356 |
| 10884831 | 9.66E−08 | ENSRNOG00000004415 |
| 10914609 | 9.71E−08 | ENSRNOG00000006736 |
| 10793708 | 9.72E−08 | ENSRNOG00000016629 |
| 10819413 | 9.80E−08 | ENSRNOG00000033854 |
| 10745504 | 9.81E−08 | ENSRNOG00000032135 |
| 10775113 | 9.84E−08 | ENSRNOG00000000075 |
| 10738671 | 9.87E−08 | ENSRNOG00000003203 |
| 10885235 | 9.95E−08 | ENSRNOG00000004873 |
| 10868315 | 1.01E−07 | ENSRNOG00000009015 |
| 10940522 | 1.01E−07 | NA |
| 10916880 | 1.01E−07 | ENSRNOG00000015133 |
| 10927538 | 1.01E−07 | ENSRNOG00000018623 |
| 10840565 | 1.02E−07 | ENSRNOG00000004641 |
| 10940501 | 1.03E−07 | NA |
| 10772863 | 1.03E−07 | ENSRNOG00000002209 |
| 10837648 | 1.04E−07 | ENSRNOG00000028215 |
| 10705104 | 1.06E−07 | ENSRNOG00000020432 |
| 10919996 | 1.06E−07 | ENSRNOG00000014201 |
| 10733004 | 1.06E−07 | ENSRNOG00000007053 |
| 10713538 | 1.08E−07 | ENSRNOG00000021206 |
| 10940271 | 1.08E−07 | ENSRNOG00000037264 |
| 10826859 | 1.09E−07 | ENSRNOG00000011384 |
| 10757438 | 1.10E−07 | ENSRNOG00000028965 |
| 10938346 | 1.10E−07 | ENSRNOG00000013322 |
| 10700445 | 1.10E−07 | ENSRNOG00000008794 |
| 10865672 | 1.11E−07 | ENSRNOG00000027466 |
| 10831162 | 1.11E−07 | ENSRNOG00000000854 |
| 10758711 | 1.11E−07 | ENSRNOG00000001351 |
| 10749648 | 1.11E−07 | ENSRNOG00000036697 |
| 10940600 | 1.12E−07 | NA |
| 10869476 | 1.12E−07 | ENSRNOG00000007886 |
| 10901045 | 1.13E−07 | ENSRNOG00000032552 |
| 10773725 | 1.14E−07 | ENSRNOG00000019624 |
| 10881685 | 1.14E−07 | ENSRNOG00000030317 |
| 10710513 | 1.14E−07 | ENSRNOG00000016960 |
| 10733139 | 1.14E−07 | ENSRNOG00000002810 |
| 10797321 | 1.15E−07 | NA |
| 10734258 | 1.15E−07 | ENSRNOG00000002599 |
| 10815888 | 1.15E−07 | ENSRNOG00000010274 |
| 10880233 | 1.15E−07 | ENSRNOG00000010892 |
| 10702165 | 1.17E−07 | ENSRNOG00000011720 |
| 10818114 | 1.17E−07 | ENSRNOG00000014002 |
| 10775769 | 1.17E−07 | ENSRNOG00000002182 |
| 10780507 | 1.19E−07 | ENSRNOG00000020482 |
| 10800760 | 1.19E−07 | ENSRNOG00000012852 |
| 10710857 | 1.20E−07 | ENSRNOG00000018604 |
| 10883716 | 1.20E−07 | ENSRNOG00000004449 |
| 10866672 | 1.20E−07 | ENSRNOG00000012602 |
| 10738056 | 1.22E−07 | ENSRNOG00000009066 |
| 10860773 | 1.22E−07 | ENSRNOG00000011175 |
| 10731385 | 1.24E−07 | ENSRNOG00000002912 |
| 10765746 | 1.25E−07 | ENSRNOG00000008045 |
| 10843918 | 1.25E−07 | ENSRNOG00000005900 |
| 10899367 | 1.27E−07 | ENSRNOG00000006934 |
| 10840142 | 1.27E−07 | ENSRNOG00000004810 |
| 10844251 | 1.28E−07 | ENSRNOG00000008258 |
| 10738676 | 1.28E−07 | ENSRNOG00000003207 |
| 10869151 | 1.28E−07 | ENSRNOG00000028063 |
| 10784054 | 1.28E−07 | ENSRNOG00000031618 |
| 10827216 | 1.29E−07 | ENSRNOG00000013771 |
| 10880056 | 1.29E−07 | ENSRNOG00000009918 |
| 10705679 | 1.31E−07 | ENSRNOG00000020505 |
| 10867657 | 1.32E−07 | ENSRNOG00000015160 |
| 10757258 | 1.32E−07 | ENSRNOG00000027028 |
| 10849172 | 1.33E−07 | ENSRNOG00000021954 |
| 10933651 | 1.33E−07 | ENSRNOG00000024792 |
| 10823508 | 1.34E−07 | ENSRNOG00000011586 |
| 10931620 | 1.34E−07 | NA |
| 10779162 | 1.35E−07 | ENSRNOG00000006619 |
| 10905765 | 1.38E−07 | ENSRNOG00000009585 |
| 10896322 | 1.38E−07 | ENSRNOG00000004704 |
| 10789688 | 1.39E−07 | ENSRNOG00000014648 |
| 10783553 | 1.40E−07 | ENSRNOG00000012046 |
| 10860528 | 1.41E−07 | NA |
| 10882905 | 1.42E−07 | ENSRNOG00000005810 |
| 10804396 | 1.42E−07 | ENSRNOG00000000158 |
| 10742555 | 1.43E−07 | ENSRNOG00000005872 |
| 10825712 | 1.44E−07 | ENSRNOG00000015274 |
| 10718344 | 1.44E−07 | ENSRNOG00000010994 |
| 10865516 | 1.45E−07 | ENSRNOG00000026309 |
| 10715268 | 1.46E−07 | NA |
| 10714537 | 1.46E−07 | ENSRNOG00000011931 |
| 10917645 | 1.47E−07 | ENSRNOG00000006864 |
| 10707626 | 1.48E−07 | ENSRNOG00000015734 |
| 10918118 | 1.48E−07 | ENSRNOG00000007926 |
| 10934585 | 1.48E−07 | ENSRNOG00000002515 |
| 10740240 | 1.49E−07 | ENSRNOG00000036664 |
| 10743468 | 1.49E−07 | ENSRNOG00000002724 |
| 10807300 | 1.50E−07 | ENSRNOG00000017674 |
| 10711251 | 1.50E−07 | ENSRNOG00000023360 |
| 10819787 | 1.51E−07 | ENSRNOG00000013011 |
| 10940250 | 1.51E−07 | ENSRNOG00000029373 |
| 10864425 | 1.52E−07 | ENSRNOG00000010301 |
| 10780937 | 1.52E−07 | ENSRNOG00000009192 |
| 10789740 | 1.53E−07 | ENSRNOG00000011276 |
| 10724449 | 1.54E−07 | ENSRNOG00000017408 |
| 10916659 | 1.55E−07 | ENSRNOG00000009243 |
| 10809604 | 1.55E−07 | ENSRNOG00000013307 |
| 10739497 | 1.56E−07 | ENSRNOG00000025669 |
| 10858686 | 1.56E−07 | ENSRNOG00000012999 |
| 10852492 | 1.56E−07 | ENSRNOG00000013267 |
| 10928524 | 1.56E−07 | ENSRNOG00000012961 |
| 10813128 | 1.57E−07 | ENSRNOG00000011105 |
| 10917448 | 1.57E−07 | ENSRNOG00000007862 |
| 10784446 | 1.59E−07 | ENSRNOG00000038762 |
| 10878112 | 1.61E−07 | ENSRNOG00000026293 |
| 10756487 | 1.61E−07 | ENSRNOG00000032759 |
| 10710993 | 1.61E−07 | ENSRNOG00000016978 |
| 10859814 | 1.62E−07 | ENSRNOG00000010702 |
| 10839790 | 1.62E−07 | ENSRNOG00000004763 |
| 10906649 | 1.63E−07 | ENSRNOG00000007065 |
| 10940611 | 1.64E−07 | NA |
| 10922286 | 1.64E−07 | ENSRNOG00000012207 |
| 10786204 | 1.64E−07 | ENSRNOG00000011636 |
| 10879879 | 1.65E−07 | ENSRNOG00000012397 |
| 10700177 | 1.65E−07 | NA |
| 10762600 | 1.66E−07 | ENSRNOG00000021871 |
| 10824434 | 1.66E−07 | ENSRNOG00000020554 |
| 10765639 | 1.66E−07 | ENSRNOG00000004761 |
| 10778661 | 1.67E−07 | ENSRNOG00000008103 |
| 10886040 | 1.68E−07 | NA |
| 10800098 | 1.69E−07 | ENSRNOG00000015332 |
| 10744410 | 1.69E−07 | ENSRNOG00000019074 |
| 10805731 | 1.69E−07 | ENSRNOG00000012853 |
| 10714338 | 1.71E−07 | ENSRNOG00000018107 |
| 10887538 | 1.73E−07 | ENSRNOG00000028566 |
| 10812270 | 1.74E−07 | ENSRNOG00000013282 |
| 10722770 | 1.74E−07 | ENSRNOG00000012716 |
| 10885618 | 1.75E−07 | ENSRNOG00000027707 |
| 10919377 | 1.76E−07 | ENSRNOG00000011501 |
| 10932211 | 1.77E−07 | ENSRNOG00000029778 |
| 10794464 | 1.77E−07 | ENSRNOG00000016468 |
| 10724895 | 1.78E−07 | ENSRNOG00000026902 |
| 10791987 | 1.78E−07 | NA |
| 10875387 | 1.79E−07 | ENSRNOG00000007445 |
| 10881468 | 1.79E−07 | ENSRNOG00000037014 |
| 10708538 | 1.81E−07 | ENSRNOG00000017289 |
| 10876594 | 1.82E−07 | ENSRNOG00000008922 |
| 10908133 | 1.84E−07 | ENSRNOG00000026643 |
| 10767987 | 1.85E−07 | ENSRNOG00000008425 |
| 10770764 | 1.85E−07 | ENSRNOG00000004789 |
| 10928902 | 1.85E−07 | ENSRNOG00000014797 |
| 10732676 | 1.86E−07 | ENSRNOG00000004217 |
| 10910015 | 1.87E−07 | ENSRNOG00000024934 |
| 10923835 | 1.88E−07 | ENSRNOG00000031232 |
| 10930435 | 1.88E−07 | ENSRNOG00000014319 |
| 10784135 | 1.89E−07 | ENSRNOG00000009849 |
| 10772200 | 1.89E−07 | ENSRNOG00000002128 |
| 10892490 | 1.90E−07 | ENSRNOG00000042613 |
| 10840005 | 1.90E−07 | NA |
| 10761025 | 1.92E−07 | ENSRNOG00000033017 |
| 10784065 | 1.92E−07 | ENSRNOG00000032868 |
| 10912908 | 1.93E−07 | ENSRNOG00000029543 |
| 10922650 | 1.95E−07 | ENSRNOG00000017660 |
| 10918368 | 1.95E−07 | ENSRNOG00000027295 |
| 10830841 | 2.01E−07 | ENSRNOG00000000763 |
| 10909733 | 2.01E−07 | ENSRNOG00000018481 |
| 10716995 | 2.02E−07 | ENSRNOG00000012885 |
| 10799397 | 2.04E−07 | ENSRNOG00000028168 |
| 10882079 | 2.05E−07 | ENSRNOG00000013820 |
| 10940623 | 2.05E−07 | NA |
| 10828863 | 2.06E−07 | ENSRNOG00000000528 |
| 10741112 | 2.07E−07 | ENSRNOG00000042086 |
| 10847754 | 2.07E−07 | ENSRNOG00000005191 |
| 10832228 | 2.08E−07 | ENSRNOG00000001201 |
| 10900651 | 2.09E−07 | ENSRNOG00000012939 |
| 10926181 | 2.09E−07 | ENSRNOG00000013368 |
| 10782919 | 2.10E−07 | NA |
| 10892648 | 2.12E−07 | ENSRNOG00000037495 |
| 10918345 | 2.14E−07 | ENSRNOG00000017714 |
| 10759538 | 2.14E−07 | ENSRNOG00000001047 |
| 10828364 | 2.15E−07 | ENSRNOG00000000461 |
| 10892859 | 2.16E−07 | NA |
| 10796970 | 2.16E−07 | ENSRNOG00000019175 |
| 10891594 | 2.18E−07 | ENSRNOG00000004707 |
| 10816712 | 2.19E−07 | ENSRNOG00000020518 |
| 10749977 | 2.19E−07 | ENSRNOG00000035524 |
| 10791677 | 2.21E−07 | ENSRNOG00000010633 |
| 10859090 | 2.24E−07 | ENSRNOG00000007866 |
| 10739313 | 2.24E−07 | ENSRNOG00000003827 |
| 10724377 | 2.24E−07 | ENSRNOG00000017191 |
| 10921527 | 2.25E−07 | NA |
| 10729791 | 2.26E−07 | ENSRNOG00000036603 |
| 10725908 | 2.26E−07 | ENSRNOG00000020217 |
| 10861399 | 2.27E−07 | ENSRNOG00000007662 |
| 10873606 | 2.27E−07 | ENSRNOG00000008052 |
| 10842409 | 2.27E−07 | ENSRNOG00000008081 |
| 10898851 | 2.27E−07 | ENSRNOG00000022778 |
| 10875959 | 2.28E−07 | ENSRNOG00000009478 |
| 10892506 | 2.32E−07 | ENSRNOG00000042654 |
| 10905248 | 2.32E−07 | ENSRNOG00000005804 |
| 10821276 | 2.33E−07 | ENSRNOG00000013177 |
| 10855416 | 2.34E−07 | ENSRNOG00000023708 |
| 10812357 | 2.35E−07 | MA |
| 10929541 | 2.37E−07 | ENSRNOG00000033747 |
| 10783510 | 2.39E−07 | ENSRNOG00000039429 |
| 10865329 | 2.39E−07 | ENSRNOG00000015411 |
| 10813191 | 2.41E−07 | ENSRNOG00000017581 |
| 10712853 | 2.41E−07 | ENSRNOG00000018752 |
| 10776915 | 2.44E−07 | ENSRNOG00000002180 |
| 10716189 | 2.44E−07 | ENSRNOG00000013993 |
| 10931537 | 2.45E−07 | NA |
| 10702768 | 2.46E−07 | ENSRNOG00000016728 |
| 10940539 | 2.46E−07 | NA |
| 10903545 | 2.48E−07 | ENSRNOG00000027690 |
| 10729350 | 2.50E−07 | ENSRNOG00000030572 |
| 10742533 | 2.50E−07 | ENSRNOG00000004956 |
| 10870316 | 2.50E−07 | ENSRNOG00000010833 |
| 10923799 | 2.53E−07 | ENSRNOG00000010283 |
| 10902564 | 2.53E−07 | ENSRNOG00000006304 |
| 10826474 | 2.54E−07 | ENSRNOG00000011929 |
| 10859296 | 2.55E−07 | ENSRNOG00000008676 |
| 10715721 | 2.57E−07 | ENSRNOG00000018704 |
| 10803394 | 2.62E−07 | ENSRNOG00000015895 |
| 10819562 | 2.65E−07 | ENSRNOG00000009205 |
| 10869050 | 2.66E−07 | ENSRNOG00000022325 |
| 10787828 | 2.66E−07 | ENSRNOG00000029441 |
| 10854957 | 2.66E−07 | NA |
| 10748200 | 2.68E−07 | ENSRNOG00000025448 |
| 10899619 | 2.68E−07 | ENSRNOG00000025891 |
| 10783805 | 2.69E−07 | ENSRNOG00000019246 |
| 10771406 | 2.69E−07 | ENSRNOG00000002217 |
| 10804132 | 2.71E−07 | ENSRNOG00000014096 |
| 10826771 | 2.72E−07 | ENSRNOG00000009877 |
| 10783501 | 2.73E−07 | ENSRNOG00000042987 |
| 10825276 | 2.74E−07 | ENSRNOG00000018220 |
| 10866507 | 2.74E−07 | ENSRNOG00000005670 |
| 10855850 | 2.75E−07 | ENSRNOG00000006858 |
| 10755846 | 2.77E−07 | ENSRNOG00000001813 |
| 10726691 | 2.83E−07 | ENSRNOG00000015593 |
| 10855405 | 2.84E−07 | ENSRNOG00000008416 |
| 10876688 | 2.87E−07 | ENSRNOG00000008618 |
| 10877916 | 2.88E−07 | ENSRNOG00000007663 |
| 10703723 | 2.92E−07 | ENSRNOG00000012179 |
| 10805540 | 2.92E−07 | ENSRNOG00000015591 |
| 10824984 | 2.93E−07 | ENSRNOG00000021120 |
| 10821194 | 2.96E−07 | ENSRNOG00000009944 |
| 10782065 | 2.98E−07 | ENSRNOG00000010989 |
| 10884961 | 3.01E−07 | ENSRNOG00000004791 |
| 10939866 | 3.01E−07 | ENSRNOG00000000869 |
| 10845070 | 3.02E−07 | ENSRNOG00000039025 |
| 10826502 | 3.03E−07 | ENSRNOG00000013798 |
| 10856166 | 3.03E−07 | ENSRNOG00000034848 |
| 10799383 | 3.07E−07 | ENSRNOG00000018091 |
| 10850668 | 3.07E−07 | ENSRNOG00000036934 |
| 10923294 | 3.08E−07 | ENSRNOG00000011677 |
| 10750848 | 3.09E−07 | ENSRNOG00000031163 |
| 10802375 | 3.11E−07 | ENSRNOG00000018770 |
| 10846293 | 3.11E−07 | ENSRNOG00000024821 |
| 10769138 | 3.14E−07 | ENSRNOG00000002736 |
| 10923996 | 3.16E−07 | NA |
| 10786454 | 3.17E−07 | ENSRNOG00000016015 |
| 10700151 | 3.19E−07 | NA |
| 10810341 | 3.20E−07 | ENSRNOG00000003496 |
| 10877907 | 3.20E−07 | ENSRNOG00000007060 |
| 10872929 | 3.24E−07 | ENSRNOG00000007992 |
| 10790811 | 3.25E−07 | ENSRNOG00000023370 |
| 10740734 | 3.28E−07 | ENSRNOG00000007117 |
| 10700759 | 3.28E−07 | NA |
| 10899579 | 3.29E−07 | ENSRNOG00000016100 |
| 10883540 | 3.31E−07 | ENSRNOG00000006865 |
| 10753024 | 3.31E−07 | ENSRNOG00000002080 |
| 10867461 | 3.33E−07 | ENSRNOG00000008180 |
| 10902868 | 3.34E−07 | ENSRNOG00000007839 |
| 10765040 | 3.36E−07 | ENSRNOG00000035336 |
| 10820362 | 3.41E−07 | ENSRNOG00000013673 |
| 10871813 | 3.41E−07 | ENSRNOG00000007629 |
| 10797863 | 3.42E−07 | ENSRNOG00000014460 |
| 10700964 | 3.46E−07 | NA |
| 10708589 | 3.49E−07 | NA |
| 10823184 | 3.50E−07 | ENSRNOG00000013434 |
| 10854494 | 3.50E−07 | ENSRNOG00000010852 |
| 10850530 | 3.51E−07 | ENSRNOG00000024799 |
| 10867944 | 3.51E−07 | ENSRNOG00000007506 |
| 10717295 | 3.55E−07 | ENSRNOG00000016219 |
| 10784074 | 3.56E−07 | ENSRNOG00000022597 |
| 10800993 | 3.56E−07 | ENSRNOG00000018775 |
| 10754000 | 3.60E−07 | ENSRNOG00000039048 |
| 10701243 | 3.60E−07 | NA |
| 10789534 | 3.66E−07 | ENSRNOG00000017085 |
| 10857385 | 3.70E−07 | ENSRNOG00000010536 |
| 10848279 | 3.75E−07 | ENSRNOG00000029847 |
| 10805375 | 3.75E−07 | ENSRNOG00000016208 |
| 10900511 | 3.75E−07 | ENSRNOG00000033262 |
| 10784679 | 3.81E−07 | ENSRNOG00000009625 |
| 10906592 | 3.81E−07 | ENSRNOG00000005291 |
| 10717014 | 3.81E−07 | ENSRNOG00000011409 |
| 10754410 | 3.81E−07 | ENSRNOG00000000399 |
| 10834406 | 3.85E−07 | ENSRNOG00000004497 |
| 10700626 | 3.85E−07 | ENSRNOG00000018091 |
| 10897952 | 3.87E−07 | ENSRNOG00000024743 |
| 10803799 | 3.88E−07 | ENSRNOG00000024241 |
| 10925060 | 3.99E−07 | ENSRNOG00000017020 |
| 10789806 | 4.01E−07 | ENSRNOG00000028380 |
| 10725819 | 4.02E−07 | ENSRNOG00000023539 |
| 10862929 | 4.02E−07 | ENSRNOG00000038346 |
| 10766674 | 4.02E−07 | ENSRNOG00000003407 |
| 10756767 | 4.06E−07 | ENSRNOG00000001099 |
| 10923857 | 4.09E−07 | ENSRNOG00000024186 |
| 10704499 | 4.10E−07 | ENSRNOG00000015865 |
| 10742674 | 4.13E−07 | ENSRNOG00000042449 |
| 10700541 | 4.14E−07 | NA |
| 10771655 | 4.17E−07 | ENSRNOG00000022256 |
| 10722315 | 4.18E−07 | ENSRNOG00000034992 |
| 10910222 | 4.18E−07 | ENSRNOG00000016905 |
| 10785602 | 4.20E−07 | ENSRNOG00000009431 |
| 10894540 | 4.23E−07 | ENSRNOG00000006978 |
| 10849765 | 4.26E−07 | ENSRNOG00000016965 |
| 10904018 | 4.27E−07 | ENSRNOG00000005145 |
| 10776976 | 4.34E−07 | ENSRNOG00000017907 |
| 10847538 | 4.34E−07 | ENSRNOG00000007732 |
| 10878221 | 4.35E−07 | ENSRNOG00000008298 |
| 10887669 | 4.38E−07 | ENSRNOG00000004268 |
| 10720437 | 4.40E−07 | ENSRNOG00000020576 |
| 10869288 | 4.41E−07 | ENSRNOG00000017132 |
| 10821340 | 4.42E−07 | ENSRNOG00000010125 |
| 10817183 | 4.46E−07 | ENSRNOG00000010105 |
| 10855135 | 4.47E−07 | ENSRNOG00000025301 |
| 10729094 | 4.47E−07 | ENSRNOG00000035384 |
| 10739984 | 4.48E−07 | ENSRNOG00000029658 |
| 10750795 | 4.55E−07 | ENSRNOG00000039567 |
| 10846631 | 4.57E−07 | ENSRNOG00000012603 |
| 10730867 | 4.58E−07 | NA |
| 10809581 | 4.64E−07 | ENSRNOG00000012153 |
| 10855576 | 4.64E−07 | ENSRNOG00000008393 |
| 10792354 | 4.66E−07 | ENSRNOG00000018241 |
| 10744134 | 4.71E−07 | ENSRNOG00000030628 |
| 10875880 | 4.71E−07 | ENSRNOG00000008314 |
| 10783763 | 4.72E−07 | ENSRNOG00000025619 |
| 10929572 | 4.74E−07 | ENSRNOG00000008664 |
| 10747439 | 4.74E−07 | ENSRNOG00000018364 |
| 10774310 | 4.74E−07 | ENSRNOG00000043210 |
| 10917727 | 4.77E−07 | ENSRNOG00000018577 |
| 10765542 | 4.77E−07 | ENSRNOG00000003551 |
| 10700290 | 4.78E−07 | NA |
| 10933298 | 4.83E−07 | ENSRNOG00000004016 |
| 10707961 | 4.89E−07 | ENSRNOG00000010964 |
| 10700897 | 4.90E−07 | NA |
| 10815795 | 4.95E−07 | NA |
| 10813236 | 4.96E−07 | ENSRNOG00000015849 |

TABLE 2
Particular Biomarkers

| affymetrix gene ID | p-value for F-statistic | ENSEMBL annotations |
|---|---|---|
| 10722208 | 1.21E−26 | ENSRNOG00000014227 |
| 10940615 | 5.06E−24 | NA |
| 10763768 | 5.25E−23 | ENSRNOG00000004441 |
| 10748273 | 5.43E−23 | ENSRNOG00000011917 |
| 10922816 | 1.22E−22 | ENSRNOG00000014378 |
| 10707142 | 1.51E−22 | ENSRNOG00000013103 |
| 10828832 | 4.58E−22 | ENSRNOG00000001869 |
| 10721261 | 7.52E−22 | ENSRNOG00000037331 |
| 10769765 | 4.07E−21 | ENSRNOG00000003136 |
| 10849279 | 1.89E−20 | ENSRNOG00000018204 |
| 10775519 | 2.50E−20 | ENSRNOG00000002159 |
| 10926252 | 2.70E−20 | NA |
| 10831940 | 1.22E−19 | ENSRNOG00000022523 |
| 10751434 | 1.44E−19 | ENSRNOG00000028918 |
| 10750524 | 4.50E−19 | ENSRNOG00000001963 |
| 10718351 | 5.16E−18 | ENSRNOG00000011174 |
| 10911315 | 5.54E−18 | ENSRNOG00000011886 |
| 10924245 | 7.72E−18 | ENSRNOG00000014269 |
| 10774383 | 9.87E−18 | ENSRNOG00000007125 |
| 10754363 | 1.32E−17 | ENSRNOG00000032728 |
| 10703666 | 1.21E−16 | ENSRNOG00000011236 |
| 10782511 | 8.04E−16 | ENSRNOG00000005883 |
| 10854847 | 2.59E−15 | ENSRNOG00000026177 |
| 10782493 | 3.48E−14 | ENSRNOG00000005883 |
| 10706308 | 7.84E−14 | ENSRNOG00000037339 |

Where a gene is identified as a “rat gene”, the present disclosure extends to a mammalian homolog thereof and in particular a human homolog. The human homolog may be referred to by a different name.

A list of proteins encoded by genes defined by Affymetrix ID number is provided in Table 3.

TABLE 3
Proteins encoded by genes defined by Affymetrix ID number

| Affymetrix ID | |
|---|---|
| Number | Protein |
| 10703666 | Pirb, paired-Ig-like receptor B |
| 10706308 | Siglec10, sialic acid binding Ig-like lectin 10 |
| 10707142 | Ldhc, lactate dehydrogenase C |
| 10718351 | Fpr1, formyl peptide receptor 1 |
| 10721261 | LOC687856, similar to myeloid cell surface antigen |
| | CD33 precursor |
| 10722208 | Mrga10, nuclear receptor Mrga10 RF-amide G protein |
| | coupled receptor |
| 10748273 | Cd79b, CD79B antigen |
| 10750524 | Mx2, myxovirus (influenza virus) resistance 2 |
| 10763768 | Faim3, Fas apoptotic inhibitory molecule |
| 10774383 | Vps54, vacuolar protein sorting 54 |
| 10775519 | LOC305166, similar to hypothelial protein 4933408F15 |
| 10782493 | RGD1564342, similar to hypothelial protein FLJ32685 |
| 10831940 | Fkbp5, FK506 binding protein 5 |
| 10849279 | Sle28a2, solute carrier family 28 [sodium-coupled |
| | nucleotide transporter] |
| 10911315 | Myole, myosin 1E |
| 10922816 | Il1r2, Interleukin 1 receptor type II |
| 10924245 | Il8rb, Interleukin 8 receptor beta |

### BRIEF DESCRIPTION OF THE FIGURES

Some figures contain color representations or entities. Color photographs are available from the Patentee upon request or from an appropriate Patent Office. A fee may be imposed if obtained from a Patent Office.

FIGS. 1a through j are graphical representations showing relationship between biomarker expression and sample number.

FIG. 2 is a graphical representation showing direct relationship between expression and time interval for the biomarker, myxovirus (influenza virus) resistance.

FIGS. 3a and b are graphical representations showing the direct relationships between expression and time for myosn1E (Myole) and sialic acid binding immunoglobulin-like lectin (Siglec10).

FIGS. 4a and b are graphical representations of RGD1564342 and Affymetrix gene is No. 10782511 at 6 hours after stroke.

FIG. 5 is a graphical representation of alpha-1-acid glycoprotein precursor expression ratios versus time. The expression ratio is the ratio between the level at a time point compared to the level at time 0 hours.

FIG. 6 is a graphical representation of alpha-2-macroglobulin precursor expression ratios versus time. The expression ratio is the ratio between the level at a time point compared to the level at time 0 hours.

### DETAILED DESCRIPTION

The present disclosure teaches biomarkers, the levels or ratios of levels of which, are useful in determining a physiological time point in the evolution of a condition or event of a vascular system such as the cerebrovascular system. The determination of a physiological time line as opposed to a “clock” time line enables better stratification of patients with respect to potential therapeutic windows. The biomarkers are selected on the basis that their levels change dynamically over time following onset of a condition or event. The expression profile of the biomarkers may also change slowly over time and reach a particular profile which equates to the level of severity of symptoms. By “change” in this context includes increasing or decreasing or both over time in a statistically consistent manner. Notwithstanding, a biomarker may also be selected on the basis that its level remains relatively constant over a time period. Such a biomarker may be used as a control or in the determination of a ratio with a dynamically changing biomarker. By “dynamically changing” means an increase or decrease in levels over time. This time period may be from minutes or hours to months or years. As taught herein, the expression profile of the biomarkers correlates to a time point or period from onset of the event or condition.

Hence, the present disclosure enables a rapid, efficient and sensitive assay for the stratification of an individual with respect to the evolution of a condition or event of a vascular system such as the cerebrovascular system. It is proposed that the physiological time period will in many cases extend beyond a clock time period. Hence, patients are more accurately stratified with respect to a therapeutic window. Within any particular pre-determined time period following onset of a condition or event, the levels of the biomarkers are determined. These levels are then used to define a physiological time period which is proposed herein to be a wider period than the corresponding “clock” time period. The levels may also be represented as a ratio with another biomarker or between two different time points. Alternatively, the pre-determined time period is based on severity of symptoms. This is more applicable to chronic conditions.

Accordingly, taught herein is a method for monitoring progression of a condition or event of a vascular system such as the cerebrovascular system in a subject, the method comprising determining the levels or ratios of levels of 2 or more biomarkers, wherein the levels of the biomarkers alter during evolution of the condition or event, wherein the levels or ratios of levels of the biomarkers relative to a control determine the extent of progression of the condition or event. In an embodiment, the biomarkers listed in Table 1 have an expression profile which correlates to a time point or period since onset of the event or condition.

The term “biomarker” is used to define a gene or gene product (protein or mRNA) which is produced or released by cells following onset of a condition or event in a vascular system such as the cerebrovascular system. Terms such as “marker”, “indicator” and “instructor” may also be used to describe a biomarker. The expression profile or presence of a profile of 2 or more biomarkers correlates to a time course following onset of the condition or event. Levels of the biomarkers may be determined by levels of mRNA transcripts or levels of proteins. Hence, the present disclosure teaches the use of transcriptomics and proteomics to define the biomarker profile. By “ratio” is meant the ratio of levels of 2 different biomarkers or the ratio of levels between one biomarker and a control marker or the ratio of levels of a biomarker at 2 different time points. A control marker may be another biomarker or a non-biomarker. An example of a control is a marker which remains relatively constant over a period of time. The expression profile of biomarkers may also be based on severity of symptoms. This may be useful in determining when therapy should be initiated.

The present disclosure teaches acute and chronic conditions or events of a vascular system such as the cerebrovascular system. By “acute” is meant that the condition or event rapidly develops over from minutes to hours to days as opposed to a “chronic” condition which may take years to develop. The biomarkers for acute conditions or events are selected on the basis that their levels change dynamically during the course of development of the acute condition or event. Chronic biomarkers may be age-based or determined based on severity of symptoms. The course of development is referred to herein as the “evolution” of the condition event.

In an embodiment, the acute condition or event is stroke. This occurs in the cerebrovascular system. Hence, the present disclosure enables a method for monitoring progression of stroke in a subject, the method comprising determining the levels or ratios of levels of 2 or more biomarkers, wherein the levels of ratios of levels of the biomarkers alter during evolution of stroke, wherein the levels or ratios of levels of the biomarkers relative to a control correlate to the extent of progression of the stroke.

In another embodiment, the chronic condition or event is vascular dementia. Accordingly, the present disclosure further teaches a method for monitoring progression of vascular dementia in a subject, the method comprising determining the levels or ratios of levels of 2 or more biomarkers, wherein the levels of ratios of levels of the biomarkers alter during evolution of vascular dementia wherein the levels or ratios of levels of the biomarkers relative to a control correlate to the extent of progression of the vascular dementia.

The term “correlate” also includes “determined”.

The stage of progression of the condition or event is determined by the levels or ratios of levels of 2 or more of the biomarkers. For thrombolytic treatment of stroke, for example, an arbitrary time of 4.5 hours has been set for safe delivery of a thrombolytic agent such as tPA. It is proposed herein, that depending on the severity of the condition or event of a vascular system such as the cerebrovascular system, and also depending on the overall health of the subject, thrombolytic intervention may be indicated at a clock time point of greater than 4.5 hours. Hence, a physiological time period is determined based on the levels or ratios of levels of 2 or more biomarkers between time 0 hours and 4.5 hours following onset. As indicated above, the “ratios” may between biomarkers, a biomarker and a control marker or a biomarker at different time points. It is proposed that thrombolytic treatment can be safely provided while the physiological time line remains within parameters determined for the equivalent clock time. The corresponding physiological time period for stroke is proposed herein to be approximately from about 0 hours to 10 hours such as 0, 0.5, 1, 1.5, 2, 2.5, 3, 3.5, 4, 4.5, 5, 5.5, 6, 6.5, 7, 7.5, 8, 8.5, 9, 9.5 or 10 hours. Similarly, a subject may present less than 4.5 hours post onset of stroke but with a biomarker expression profile instructive as to greater than 4.5 hours. Such a subject may then not be deemed eligible for tPA treatment.

For treatment of other conditions such as vascular dementia, the expression profile of the biomarkers may be determined in a control subject having a certain level of severity of symptoms. The levels or ratios of levels of biomarkers then equate to that level of severity of symptoms. A subject with such an expression profile of biomarkers may then require therapeutic intervention.

Hence, enabled herein is a method for monitoring progression of a thromboembolic condition or event of a vascular system such as the cerebrovascular system in a subject, the method comprising determining the levels or ratios or levels of 2 or more biomarkers, the biomarkers selected on the basis that in the time period since onset of the thromboembolic condition or event, the levels or ratios of levels of the biomarkers are instructive as to a time point along a time line, wherein the levels or ratios of the levels of the biomarkers relative to a control correlate to the extent of progression of the condition or event.

Another aspect taught herein is a method for monitoring progression of a chronic condition or event of a vascular system such as the cerebrovascular system in a subject, the method comprising determining the levels or ratios or levels of 2 or more biomarkers, the biomarkers selected on the basis that a particular profile is obtained which equates to the severity of symptoms of the condition or event, wherein the levels or ratios of the levels of the biomarkers relative to a control correlate to the extent of progression of the condition or event.

Aspects taught herein are predicated in part on the determination that the ischemia or hemorrhage within a vascular system such as the cerebrovascular system triggers a rapid and stereotyped acute phase response which is used to establish biological time points since onset of the ischemic or hemorrhagic condition or event. Cell death, for example, releases cell contents into the blood stream. It is taught herein to replace the clock time points with a physiological time line based on the profile of levels of selected biomarkers. Although this is particularly applicable to ischemic conditions or events of a vascular system such as the cerebrovascular system, it also applies to hemorrhagic conditions or events.

Another aspect enabled herein is a method for stratifying a subject with respect to progression of a condition or event of a vascular system such as the cerebrovascular system, the method comprising determining the levels or ratios of levels of 2 or more biomarkers, the levels or ratios of levels of which alter over time from onset of the condition or event wherein the combination of the 2 or more levels or ratios of levels establishes a physiological time line which, when compared to a control, enables identification of the extent of progression of the condition or event. The physiological time line may also equate to severity of symptoms.

The extent of progression is important in order to define certain therapeutic windows. For ischemic conditions involving an occlusion, administration of a thrombolytic agent is recommended within a 4.5 hour time period since onset. However, the exact time point of onset may not be known with any accuracy. Hence, the levels or ratios of levels of biomarkers or the biomarker profile is used to determine physiologically the likely time period since onset. For chronic conditions, the biomarker expression profile can be used to determine when a subject should receive treatment or when the treatment regime should change.

By “condition” or event of a vascular system such as the cerebrovascular system” is meant any disease or physiological phenomenon arising from an ischemic incident or hemorrhage in a vascular system. Examples of such conditions and events include stroke, myocardial ischemia and infarction and cardiomyopathies including ischemic cardiomyopathy and ischemic heart disease as well as vascular dementia. In an embodiment, the condition or event occurs in the cerebrovascular system such as ischemic or hemorrhagic stroke. In an embodiment, the condition or event is ischemic stroke since it is critical to know the period of progression of the stroke to establish whether the patient is still within the therapeutic window for thrombolytic intervention. In another embodiment, the condition is vascular dementia which may take many years to develop. However, it is also critical to assist clinicians in determining when therapy needs to be initiated or altered.

In an embodiment, the condition or event of a vascular system such as the cerebrovascular system is of ischemic origin wherein thrombolytic intervention has the potential to alleviate the occlusion which led to the ischemic event. The present disclosure further teaches using the profile of expression of the biomarkers to distinguish between conditions and events such as between ischemic and hemorrhagic stroke. Hence, the profile of biomarkers may change depending on the condition or event.

“Stratification” includes identification, diagnosis, prognosis, clarification, monitoring and/or determination of the presence, level, severity, state and/or classification of an acute condition or event of a vascular system such as the cerebrovascular system. In an embodiment, the subject is stratified with respect to progression of the acute condition or event from onset. Generally, this is based on comparing a knowledge base of levels or ratios of levels of biomarkers in body fluid including plasma, whole blood and serum to another knowledge base of pre-determined levels, statistically correlated to the evolution of the condition or event. The correlation may be based on a pre-determined time period or on a pre-determined level of severity of symptoms.

Hence, the present disclosure teaches the identification of a correlation between the levels or ratios of levels of particular biomarkers and evolution of the condition or event since onset. In an embodiment, the levels of biomarkers are determined at clock time points such as at but not limited to between 0 hours and 4.5 hours following onset of the condition or event. This information becomes a control knowledge base of data. Determination of the levels of the biomarkers by a patient provides a test knowledge base of data. Comparison of the test knowledge base with the control knowledge base enables an estimate of the likely point in the evolution of the condition or event. In another embodiment, the biomarker expression profile is correlated to severity of symptoms.

As indicated above, a condition or event of a vascular system such as the cerebrovascular system includes stroke (ischemic or hemorrhagic stroke) and cardiovascular disease such as ischemic mitochondrial infarction, and organ damage due to ischemic conditions. Those conditions are regarded as being acute. In a particular embodiment, the condition or event is ischemic stroke. The term “ischemic stroke” as used herein is to be considered as an individual condition as well as a spectrum of conditions including a range of risk indicators of the level of disease progression. This risk ranges from minor to extreme. The ability to monitor and identify markers of stroke enables decisions on which type of medical intervention is required from behavioural modification and medicaments to surgical intervention. This is particularly the case for monitoring the window of thrombolytic intervention. The physiological time line is proposed herein to define a therapeutic window for thrombolytic intervention which is wider than the clock time window of 4.5 hours.

The present disclosure teaches any or all conditions within the clinical spectrum of “ischemic stroke” such as pre-stroke and ischemic attacks.

In another embodiment, the condition or event is vascular dementia, Alzheimer's disease, Parkinson's disease or other conditions resulting in dementia. Such conditions are regarded as chronic. The physiological time line then matches severity of symptoms or may be age-based.

Reference herein to a “subject” includes a human which may also be considered an individual, patient, host, recipient or target. The subject may also be an animal, such as used in an animal model.

Useful biomarkers contemplated for use herein include 2 or more biomarkers selected from those listed in Table 1. In an embodiment, the biomarkers include 2 or more of the biomarkers listed in Table 2. Tables 1 and 2 list biomarkers useful in the practice of the present disclosure by Affymetrix gene identifier (ID) number (Affymetrix gene identifier). Information on the genes can be obtained from the Affymetrix website [NetAffx (Trademark)] which can be accessed at http://www.affymetrix.com/analvsis/index.affx. Where available, an ENSEMBL identifier is also provided which can be accessed through the ENSEMBL rat genome website at http://www.ensembl.org/Rattus_norvegicus. Where a gene is identified as a “rat gene”, the present disclosure extends to a mammalian homolog thereof and in particular a human homolog. The human homolog may be referred to by a different name. The term “biomarker” extends to both the gene or gene product such as a protein or mRNA transcript.

By name, particularly useful biomarkers include alpha-1-macroglobulin precursor, alpha-2-macroglobulin precursor, CINC-1, myxovirus (influenza virus) resistance 2, nuclear receptor MrgA10 RF-amide G protein-coupled receptor, lactate dehydrogenase C, FK506 binding protein 5, interleukin 1 receptor type II, interleukin 8 receptor beta formyl peptide receptor 1, paired-immunoglobulin-like receptor 8, myeloid cell surface antigen CD33 precursor (Siglec), lute carrier family 28 (sodium-coupled nucleoside transporter) and vacuolar protein sialic acid binding immunoglobulin-like lectin 10.

In an embodiment, the levels or ratios of levels of between about 2 and about 2054 biomarkers are determined from Table 1. In an embodiment, the biomarkers may be reviewed as sub-groups of from 2 to 500 or 2 to 100 or 2 to 50 biomarkers. In an embodiment, the levels or ratios of levels of from about 2 to 20 biomarkers are determined including about 8 to 15 biomarkers such as about 12 biomarkers. Selection of groups of biomarkers may be made based on patient data and/or the condition or event being diagnosed or treated. In an embodiment, for about 2 to about 25 biomarkers are selected from the list set forth in Table 2, including from about 8 to 15 such as 12 biomarkers.

The biomarker profile determined may also be selected based on the age, weight, sex, overall physical wellbeing or other parameters of the subject such as severity of symptoms. Hence, a profile of biomarkers may be selected in a personal medicine approach to establish the physiological time line of the condition or event.

The present disclosure enables, therefore, a physiological time line to be determined for a subject based on biomarker profile which in turn is instructive as to which therapeutic intervention protocol is the most appropriate. The stratification or profiling enables early diagnosis, confirmation of a clinical diagnosis, treatment monitoring and treatment selection as well as determining whether a subject is within a therapeutic window for thrombolytic or other treatment treatment.

Another aspect taught herein is an assay to identify whether a subject can be given thrombolytic intervention following onset of an ischemic condition or event in a vascular system such as the cerebrovascular system, the method comprising determining levels or ratios of levels of 2 or more biomarkers which correlate with evolution of the condition or event following onset at time 0 hours and 4.5 hours wherein a comparison of the biomarker levels or ratios of levels with a control enable determination of whether the subject is within the thrombolytic intervention therapeutic window.

The disclosure enables a method of treating a subject who has or is suspected of having suffered an acute ischemic condition or event of a vascular system such as the cerebrovascular system, the method comprising determining the levels or ratios of levels of 2 or more biomarkers, wherein the levels or ratios of levels of the biomarkers alter during evolution of the condition or event, wherein the levels or ratios of levels of the biomarkers relative to a control determine the extent of progression of the condition or event and where the levels of the biomarkers approximately equate to the levels present from 0 hours to 4.5 hours after onset of the condition or event and administering to the subject a thrombolytic agent. If the levels or ratios of levels of the biomarkers indicate that physiologically the subject is within the 0 to 4.5 hour time period from onset.

The present disclosure is instructional for a method of treating a subject who has or is suspected of having suffered a stroke the method comprising determining the levels or ratios of levels of 2 or more biomarkers, wherein the levels or ratios of levels of the biomarkers alter during evolution of the stroke, wherein the levels or ratios of levels of the biomarkers relative to a control determine the extent of progression of the stroke and where the levels or ratios of levels of the biomarkers approximately equate to the levels present from 0 hours to 4.5 hours after onset of the stroke, administering to the subject a thrombolytic agent. If the levels or ratios of levels of the biomarkers indicate that physiologically the subject is within the 0 to 4.5 hour time period from onset.

Yet another aspect taught herein is a method of treating a subject the method comprising determining the levels or ratios of levels of 2 or more biomarkers, wherein the levels or ratios of levels of the biomarkers alter during evolution of a condition or event wherein the levels or ratios of levels of the biomarkers equates to a level of severity of symptoms, wherein the levels or ratios of levels of the biomarkers relative to a control determine the extent of progression of the condition or event and then providing treatment to the subject when the levels or ratios of levels equate to a set severity of symptoms.

The physiological time period corresponding to the levels or ratios of levels of the biomarkers pre-determined between 0 and 4.5 hours is wider than the clock time period. Hence, more patients will qualify for thrombolytic treatment. Notwithstanding, the present disclosure teaches the identification of patients having a biomarker expression profile of >4.5 hours post onset who would be contraindicated for thrombolytic treatment.

Yet another aspect enabled herein is the use of 2 or more biomarkers, the levels of which dynamically alter during evolution of an acute condition or event of a vascular system such as the cerebrovascular system in a subject, in the manufacture of a diagnostic assay to determine the extent of progression of the condition or event.

As indicated above, the 2 or more biomarkers are generally selected from the list in Table 1. In an embodiment, the 2 or more biomarkers are selected from the list in Table 2.

Provided herein, therefore, is a panel of biomarkers, expression of which corresponds to the extent of progression of a condition or event of a vascular system such as the cerebrovascular system.

In an embodiment, temporal changes in biomarkers following ischemic or hemorrhagic stroke are as follows:

Steady decline with time of onset
10750524 (Mx2, myxovirus (influenza virus) resistance 2)
Steady increase to 6 hours from onset then falling
10940615
10926252

### CINC-1

Rapid early rise then rapid fall by 6 hours from onset
10722208 (Mrga 10, nuclear receptor Mrga10 RF-amide G protein-coupled receptor)
10707142 (Ldhc, lactate dehydrogenase C)
10831940 (Fkbp5, FK506 binding protein 5)
10828832
Rapid early rise then persistent high expression to 6 hours from onset followed by slow fall by 24 hours
10922816 (Il1r2, Interleukin 1 receptor, type II)
10924245 (Il8rb, Interleukin 8 receptor, beta)
10718351 (Fpr1; formyl peptide receptor 1)
10854847
10703666 (Pirb, paired-Ig-like receptor B)
10721261 (LOC687856, similar to myeloid cell surface antigen CD33 precursor)
Steady rise to 6 hours, normalised by 24 hours from onset
10775519 (LOC305166, similar to hypothetical protein 4933408F15)
10849279 (Slc28a2, Solute carrier family 28 (sodium-coupled nucleoside transporter))
10774383 (Vps54, Vacuolar protein sorting 54 [yeast])
10754363
10751434
Rapid fall by 1 hour, persistent suppression to 6 hours, then normalising by 24 hours from onset
10763768 (Faim3, Fas apoptotic inhibitory molecule)
10748273 (Cd79b, CD79B antigen)
10769765
10911315 (Myo1e, myosin 1E)
10706308 (Siglec10 sialic acid binding Ig-like lectin 10)
No change of expression to 3 hours then increase by 6 hours from onset
10782493 (RGD1564342, similar to hypothetical protein FLJ32685)
10782511
Alpha-1-acid glycoprotein precursor
No change of expression to 6 hours then increase by 24 hours from onset
Alpha-2-macroglobulin precursor

There are many methods which may be used to detect the levels of the biomarkers including determination of mRNA expression levels and/or protein levels such as by immunological means or by mass spectrometry including liquid chromatography and electrospray ionization-tandem mass spectrometry.

Immunological assays for the biomarkers may be in any convenient format as known in the art. These include Western blots, immunohistochemical assays and ELISA assays. Any means for detecting a level of a biomarker of the expression or protein level can be used in accordance with the present disclosure. Any one of a range of genetic assays may be conducted to quantitative mRNA levels and expression level including quantitative PCR. In an embodiment, microarrays of genetic molecules or immunological agents such as antibodies specific for biomarkers are employed. In one aspect, a solid phase support is provided comprising ligands which are capable of capturing the biomarkers. The solid phase support may be in the form of a chip, microarray, dipstick, micro- or nano-titer plate with wells and the like. The solid phase microarray may measure levels or changes in expression levels, velocities, concentrations or activities of the biomarkers to thereby provide a profile of biomarker expression. The ligands include antibodies, nucleic acid probes, receptors or other molecule capable of capturing a biomarker. The solid phase array may also be interfaced with a computer to assist in the analysis and/or generation of data. In an embodiment, the levels or ratios of levels of the biomarkers are subjected to multivariate analysis in a multivariate statistical model.

The biological sample is any fluid or cell or tissue extract in a subject which comprises the biomarker. In another embodiment, the biological sample includes serum, whole blood or blood plasma, lymph, urine, saliva or a cell extract. Generally, samples are analyzed at point of care facilities or analyzed remote from the point of care facility but report to the facility such as by a web-based reporting system.

In an embodiment, the present disclosure teaches identification of the presence of biomarkers associated with the evolution of an ischemic condition or event within a vascular system such as the cerebrovascular system which is instructive as to whether a subject is eligible for thrombolytic intervention. In order to detect the levels of the biomarkers, a biological sample is prepared and analyzed for a difference in levels or ratios of levels between the subject being tested and a control. In this context, a “control” includes the levels in a statistically significant comparable population free of a condition or event or following the condition or event. Conveniently, the control is a knowledge database which comprises statistically validated levels or ratios of levels of the biomarkers at pre-determined the points such as between time 0 hours and the 4.5 hours following onset of the condition or event. The time points may be taken at any time between the 0 hour to 4.5 hour range such as at 0, 30 minutes, 60 minutes, 90 minutes, 120 minutes, 180 minutes, 210 minutes, 240 minutes and 270 minutes, or at any points inbetween. Alternatively, the control is based on severity of symptoms in a control subject. At various stages during progression of a condition or event, a biomarker expression profile is determined.

The identification of the association between the pathophysiology of a condition or event of a vascular system such as the cerebrovascular system and levels of, or ratios of, biomarkers permits the early screening of individuals to identify those who may benefit from thrombolytic intervention or other form of therapy. The subject assay enables practitioners to identify or stratify individuals who are within the therapeutic window for thrombolytic intervention based on physiological means rather than just “clock time” and who may benefit from this treatment. This treatment may also be complemented with certain behavioral or therapeutic or dietary protocols to reduce the risk of further exacerbating vascular disease. Furthermore, for more chronic conditions, the biomarker expression profile can assist a clinician to decide when therapy should be initiated or changed.

Even yet another aspect of the present disclosure teaches a method of treatment of a subject comprising assaying the subject with respect to the levels or ratios of levels of 2 or more biomarkers which levels or ratios of levels define the evolution of a condition or event of a vascular system such as the cerebrovascular system wherein the levels or ratios of levels of the biomarkers relative to a control provides a correlation to the presence, state, classification or progression of the condition or event and then providing therapeutic intervention to the subject based on the progression of the condition or event.

In an embodiment, the present disclosure enables a method of treatment of a subject comprising assaying the subject with respect to the levels or ratios of levels of 2 or more biomarkers which define the evolution of a stroke wherein the levels or ratios of levels of the biomarkers relative to a control provide a correlation to the presence, state, classification or progression of the stroke and then providing therapeutic intervention to the subject based on the progression of the stroke.

Enabled herein is a method for the treatment of a neurological condition such as a stroke or symptoms of stroke by determining the extent of progression of an acute condition or event of the cerebrovascular system leading to the neurological condition and providing therapeutic intervention based on the extent of progression. Examples of therapeutic intervention include administration of a thrombolytic agent, oral aspirin, surgical intervention and/or behavoral and/or care intervention.

A web-based system is provided herein where data on expression levels of biomarkers associated with the evolution of a condition or event of a vascular system such as the cerebrovascular system are provided by a client server to a central processor which analyzes and compares to a control and optionally considers other information such as patient age, sex, weight and other medical conditions and then provides a report, such as, for example, a risk factor for disease severity or progression or status or an index of probability that the condition or event is at a certain stage or time from onset. The report is conveniently provided to a clinician at a point of care facility.

Hence, knowledge-based computer software and hardware are also taught by the present disclosure.

In an embodiment, the assay enabled herein may be used in existing or newly developed knowledge-based architecture or platforms associated with pathology services. For example, results from the assays are transmitted via a communications network (e.g. the internet) to a processing system which generates a physiological time period, based on pre-determined data, which is then forwarded to an end user in the form of a diagnostic or predictive report. In an embodiment, for example, the report specifies whether the patient is within a particular the therapeutic window. One such therapeutic window is the time period for thrombolytic intervention such as in the case of ischemic stroke.

The assay may, therefore, be in the form of a kit or computer-based system which comprises the reagents necessary to detect the concentration of the biomarkers directly or via expression of genes and the computer hardware and/or software to facilitate determination and transmission of reports to a clinician at a point of care facility.

A method is taught herein of allowing a user to determine the status of a subject with respect to an acute condition or event of a vascular system such as the cerebrovascular system, the method including receiving data, in the form of levels or ratios of levels of 2 or more biomarkers associated with the evolution of the condition or event; wherein the levels or ratios of levels of the biomarker relative to a control provide a correlation to the presence, state, classification or progression of the condition or event, from the user via a communications network which processes the data and which determines the status of the subject in accordance with a comparison with pre-determined values, wherein the results indicating the status of the subject are transferred to the user via the communications network. The “status” is generally the predicted time point following onset. The 2 or more biomarkers are selected from those listed in Table 1 or Table 2.

Conveniently, the method generally further includes having the user determine the data using a remote end station, and transferring the data from the end station to the base station via the communications network.

The base station can include first and second processing systems, in which case the method can include transferring the data to the first processing system, transferring the data to the second processing system, and causing the first processing system to perform the processing to generate the an estimate of the time since onset.

The second processing system may also be coupled to a database adapted to store pre-determined data which queries the database to obtain at least selected pre-determined data from the database and compares the selected pre-determined data to the subject data or generating a predicted probability index that the condition or event as progressed to a certain extent.

The present disclosure also teaches a base station for determining the status of a subject with respect to an estimated physiological time period since onset, the base station including:

(a) a data storage facility;

(b) a data processing system, the processing system being adapted to receive subject data from a user via a communications network, the data including levels or ratios of levels of biomarkers associated with the evolution of a condition or event of a vascular system such as the cerebrovascular system, comparing the data to pre-determined data and determining the status of the subject in accordance with the results of the processing including the comparison; and

(c) an output function to provide an indication of the status of the subject to the user via the communications network.

In an embodiment, an algorithm is used to compare test data with pre-determined data. The algorithm may perform univariate or multivariate statistical analysis.

The processing system can be adapted to receive data from a remote end station adapted to determine the data.

In accordance with this embodiment, levels or ratios of levels of the biomarkers may be screened alone or in combination with other biomarkers of a condition or event. An “altered” level means an increase or elevation or a decrease or reduction in the concentrations of the biomarkers or their velocities or level of expression of a gene. It is proposed herein that the levels or ratios of levels of the biomarkers are indicative of extent of progressive or evolution of condition or the event.

The determination of the concentrations or levels or velocities or ratios of levels of the biomarkers enable establishment of a diagnostic rule based on the concentrations, levels, velocities or ratios relative to controls. Alternatively, the diagnostic rule is based on the application of a statistical and machine learning algorithm. Such an algorithm uses relationships between levels or ratios of levels biomarkers and disease status observed in training data (with known disease status) to infer relationships which are then used to predict the status of patients with unknown status. An algorithm is employed which provides an index of probability that a patient has had a condition or event within a certain time period. The algorithm would perform in an embodiment, a univariate or multivariate analysis function. In a particular embodiment, the training data comprise the levels or ratios of levels of the biomarkers within a pre-determined time period. In an embodiment, this time period is from about 0 hours to about 4.5 hours. Thereafter, the time period is estimated based on a physiological time period which is proposed to be longer than the clock time period of, for example, 0 to 4.5 hours. Hence, more patients will likely qualify for a particular treatment associated with this physiological time period.

Hence, a diagnostic rule is enabled herein based on the application of statistical and machine learning algorithms. Such an algorithm uses the relationships between levels or ratios of levels of biomarkers and in training data to infer relationships which are then used to predict the time period in patients with unknown status. Practitioners skilled in the art of data analysis recognize that many different forms of inferring relationships in the training data may be used without materially changing the present disclosure.

Hence, the present disclosure is instructional on the use of a knowledge base of training data comprising levels or ratios of levels of biomarkers associated with the evolution of an acute condition or event of a vascular system such as the cerebrovascular system in a subject over a clock time period to generate an algorithm which, upon input of a second knowledge base of data comprising levels of the biomarkers from a patient with an unknown status of time of onset, provides an index of probability that predicts the likely time period since onset. Alternatively, the training data comprise a biomarker expression profile based on level of severity of symptoms. Hence, the algorithm then provides an index of probability that a patient has reached a certain stage of disease progression. This information is then useful to determine windows for appropriate therapeutic intervention.

The term “training data” includes knowledge of levels or ratios of the biomarkers factors relative to a control. A “control” includes a comparison to levels of the biomarkers in a subject with known onset time of the condition or event.

Hence, the “training data” includes levels or ratios of 2 or more biomarkers.

Data generated from the levels of the biomarkers are input data. The input of data comprising the levels of the biomarkers are compared with a control or is put into the algorithm which provides a risk value of the likelihood that the subject being in a defined therapeutic window or time period. A treatment regime is then selected such as based on whether the subject is eligible for thrombolytic intervention, aspirin, management or surgical intervention.

In an embodiment, a method is enabled for monitoring the progression of a condition or event of a vascular system such as the cerebrovascular system in a patient, comprising:

(a) providing a sample from a patient;

(b) determining the levels or ratios or levels of 2 or more biomarkers wherein the levels or ratios of the biomarkers relative to a control provide a correlation to the presence, state, classification or progression of the condition or event;

(c) repeating steps (a) and (b) at a later point in time and comparing the result of step (b) with the result of step (c) wherein a difference in the index of probability is indicative of the progression of the condition in the patient. In an embodiment, the condition or event is a stroke.

Reference to an “algorithm” or “algorithmic functions” as outlined above includes the performance of a univariate or multivariate analysis function. This includes the determination of an F-statistic or a p-value associated with an F-statistic. A range of different architectures and platforms may be implemented in addition to those described above. It will be appreciated that any form of architecture suitable for implementing the present disclosure may be used. However, one beneficial technique is the use of distributed architectures. In an embodiment, a number of end stations may be provided at respective geographical locations. This can increase the efficiency of the system by reducing data bandwidth costs and requirements, as well as ensuring that if one base station becomes congested or a fault occurs, other end stations could take over. This also allows load sharing or the like, to ensure access to the system is available at all times.

In this case, it would be necessary to ensure that the base station contains the same information and signature such that different end stations can be used.

It will also be appreciated that in one example, the end stations can be hand-held devices, such as PDAs, mobile phones, or the like, which are capable of transferring the subject data to the base station via a communications network such as the Internet, and receiving the reports. Hence, point of care facilities can be remote from where the levels of the biomarkers are determined.

In the above aspects, “data” mean the levels, ratios of levels, concentrations, velocities or level of transcription of the biomarkers. The “communications network” includes the internet. When a server is used, it is generally a client server or more particularly a simple object application protocol (SOAP).

A report outlining the likelihood of time of onset of the condition or event is then issued.

The present disclosure further teaches the use of the levels of ratios or levels of 2 or more biomarkers listed in Table 1 or Table 2 to determine the extent of progression of a condition or event of a vascular system such as the cerebrovascular system.

Aspects of the present disclosure are described by the following non-limiting Examples.

### Example 1

### Identification of Biomarkers

Biomarkers are identified by expression analysis versus selected time points after onset of a stroke in a rat stroke model. mRNA was extracted from whole blood of rats which had an induced stroke. mRNA was measured using Affymetrix (Trademark) rat gene expression arrays of type RaGene-1-O-st-v1. The data were statistically analyzed using linear models such as those implemented in the LIMMA package (Smyth et al. (2005) LIMMA:Linear Models for microarray data In Bioinformatics and Computational Biology Solutions using R and Bioconductor R. Gentleman V Carey, S. Dudoit, R Irizarry W Huber (Eds), Springer, New York: 397-420) The list of biomarkers is provided in Table 1.

In an embodiment, the biomarkers are listed in Table 2. The present disclosure extends to multiple subsets of biomarkers from Table 1 or Table 2 based on patient data (disease history, sex, weight, age, etc) or on the condition or event being diagnosed or treated.

The biomarkers in Tables 1 and 2 are listed by Affymetrix gene identifier (ID) number (Affymetrix gene identifier). The biomarkers were selected with a p-value cutoff over all time points of 5×10⁻⁶. Generally, 6 time points were used at which to measure the levels of the biomarkers. The order of the genes is by p-value of the F-statistic from decreasing order of significance. Information on the genes can be obtained from the Affymetrix website [NetAffx (Trademark)] which can be accessed at http://www.affymetrix.com/analysis/index.affx. Where available, an ENSEMBL identifier is also provided which can be accessed through the ENSEMBL rat genome website at http://www.ensembl.org/Rattus_norvegicus. The term “NA” is used when the ENSEMBL identifier is not available. Where a gene is identified as a “rat gene”, the present disclosure extends to a mammalian homolog thereof and in particular a human homolog. The human homolog may be referred to by a different name.

### Example 2

### Expression of Biomarkers

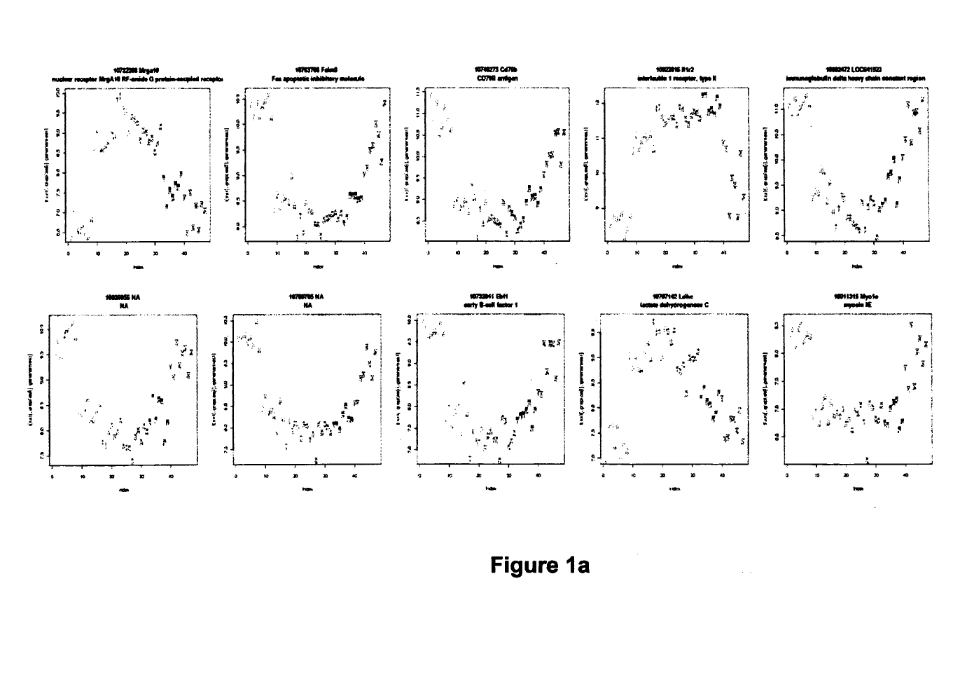

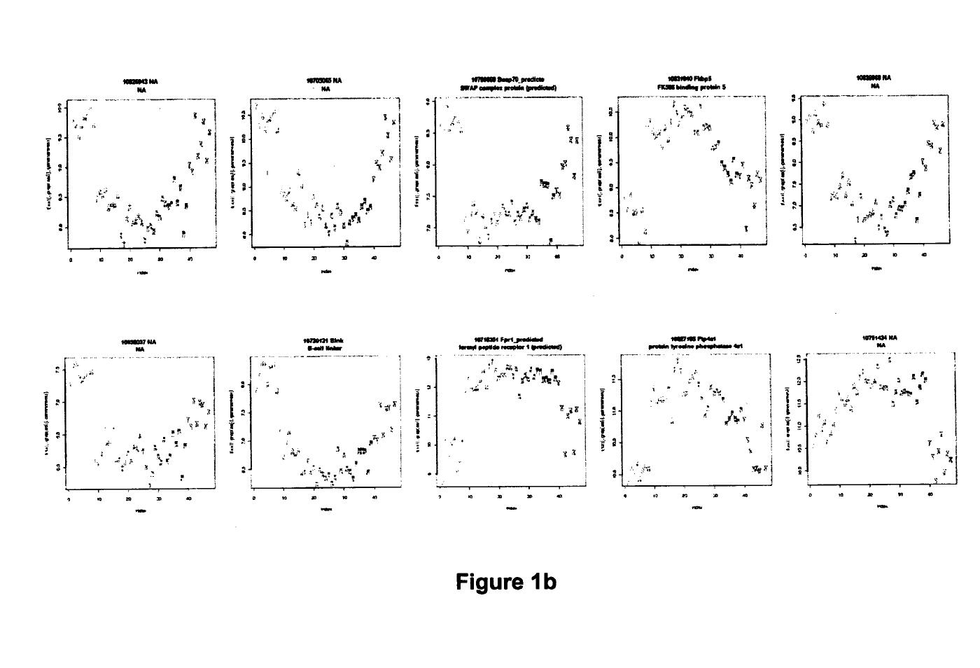

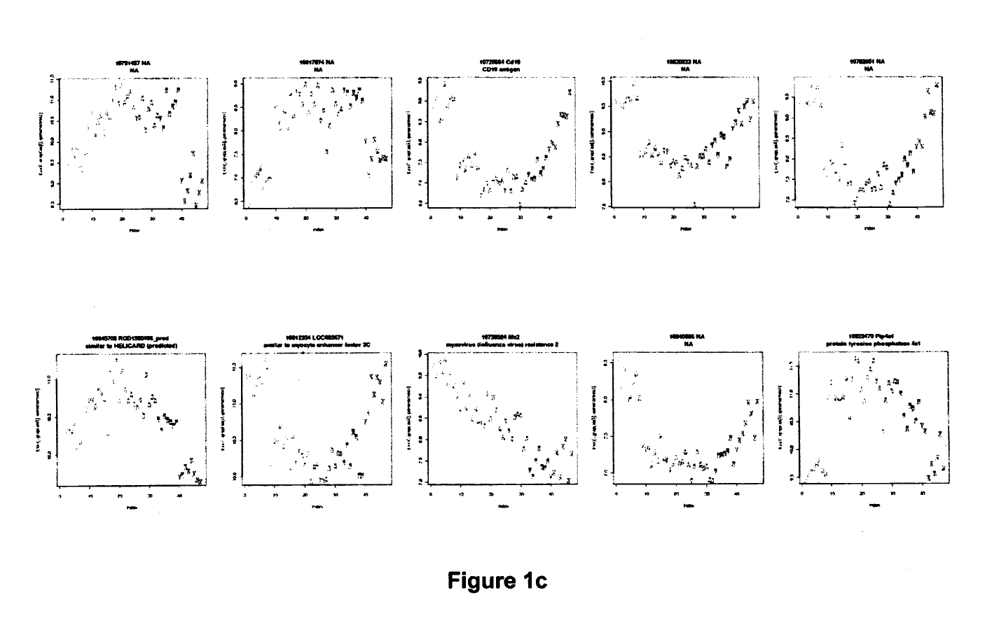

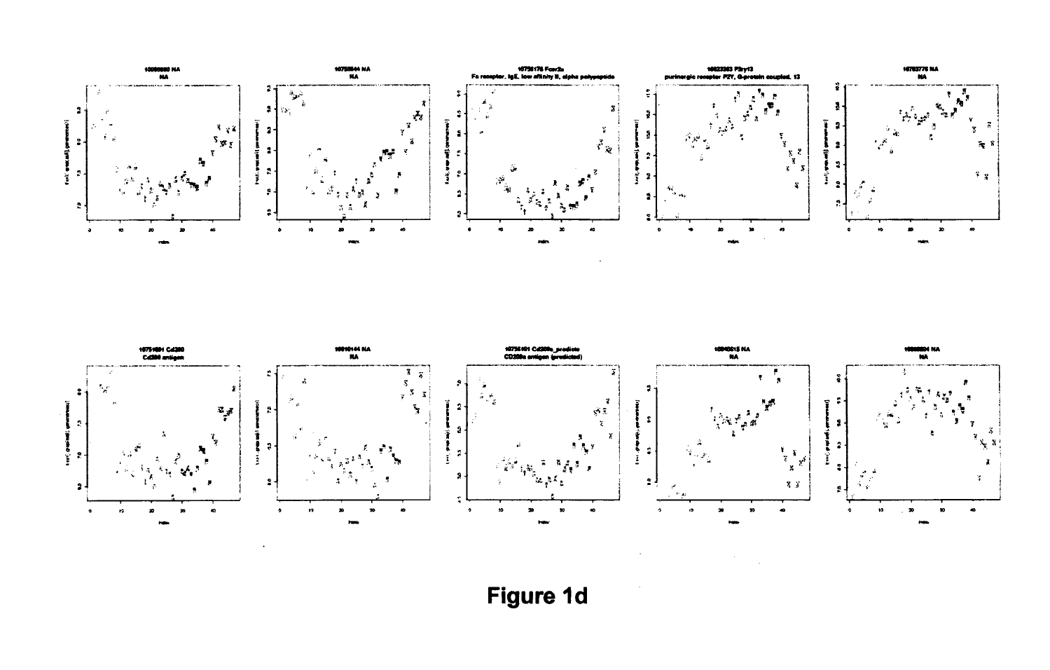

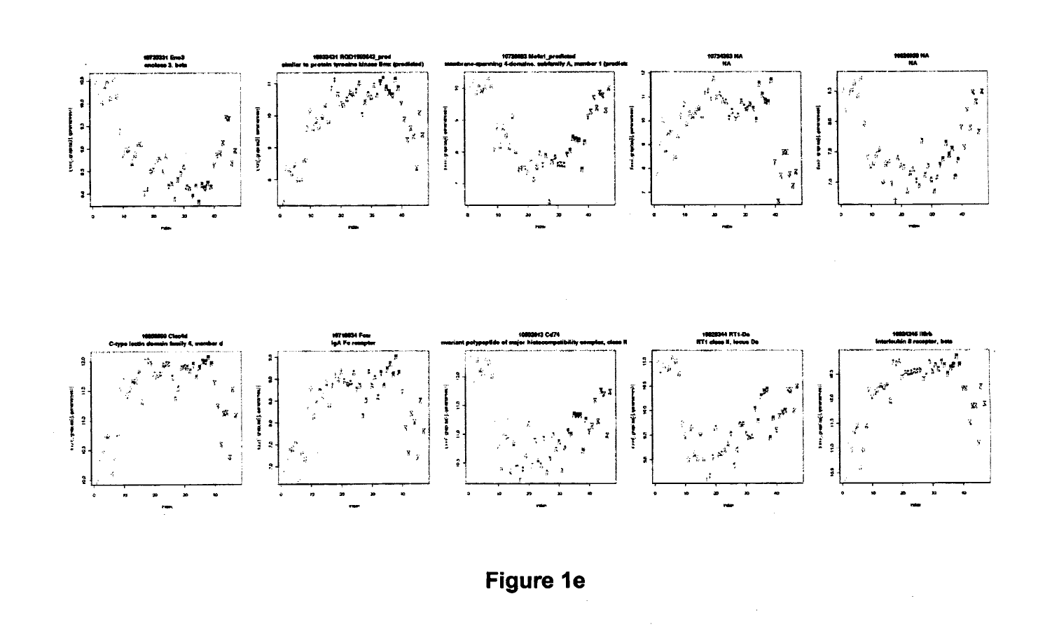

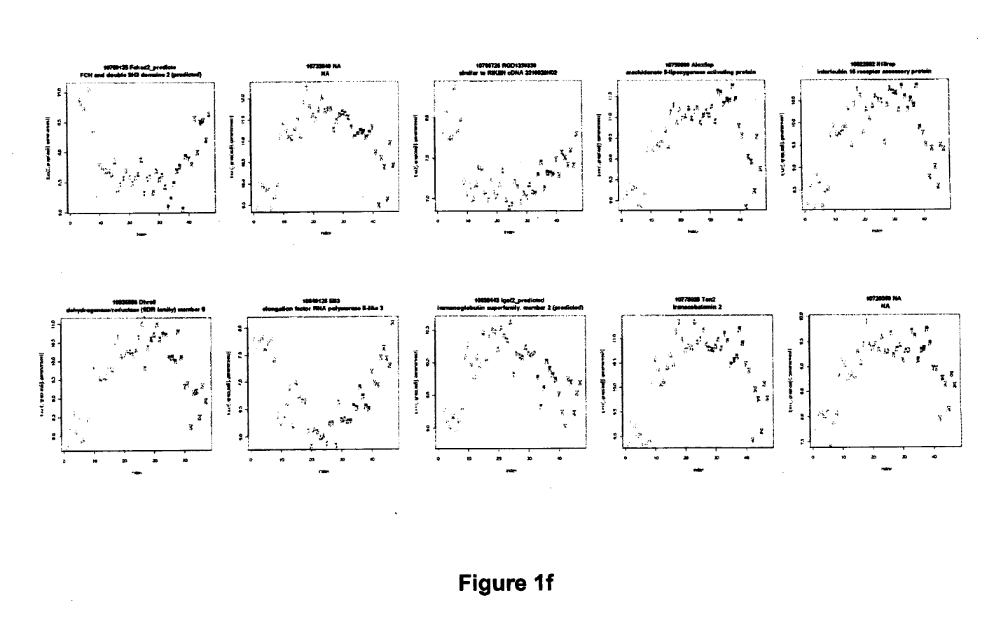

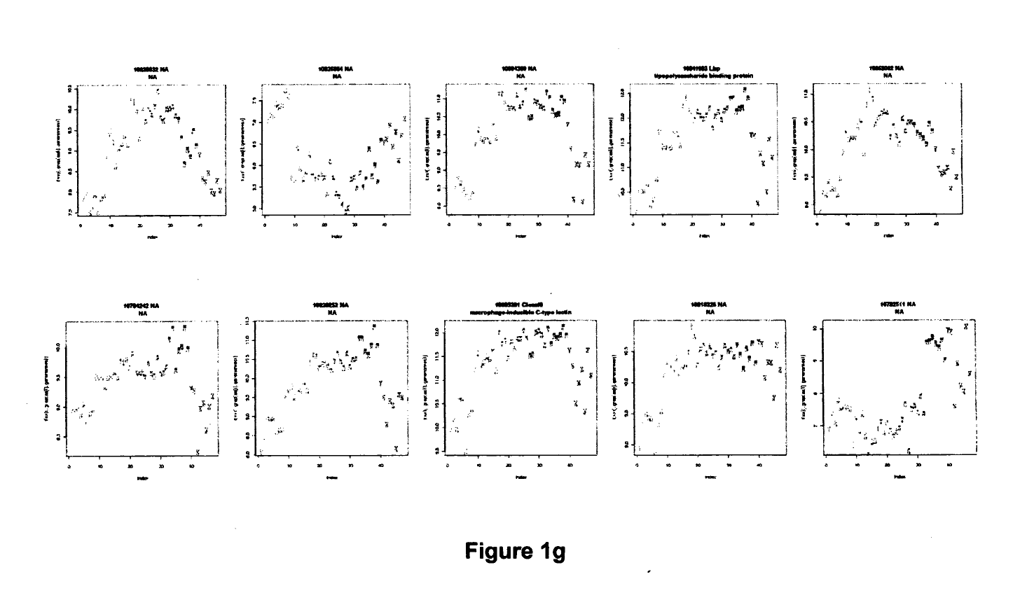

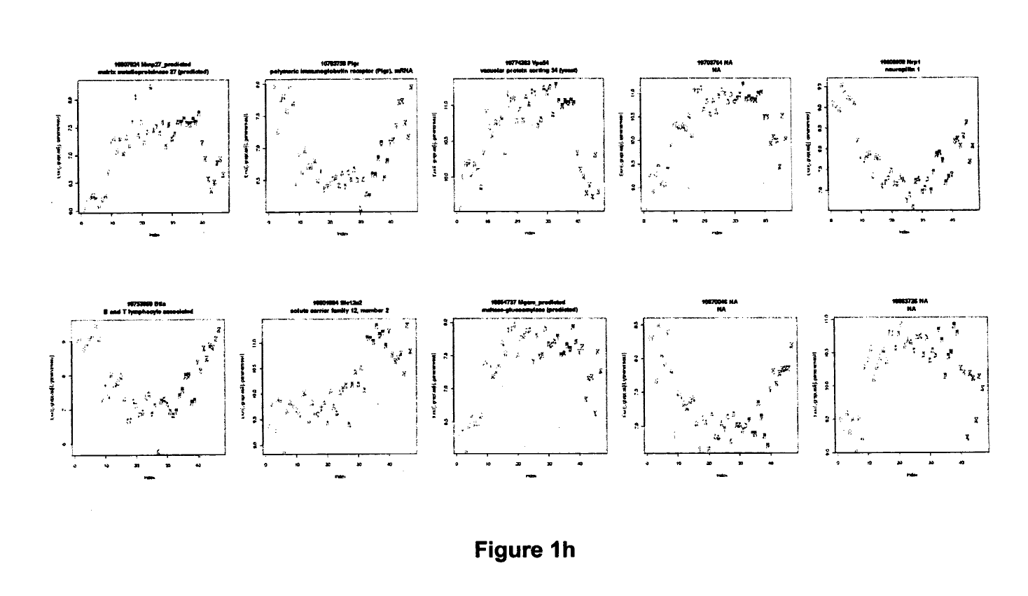

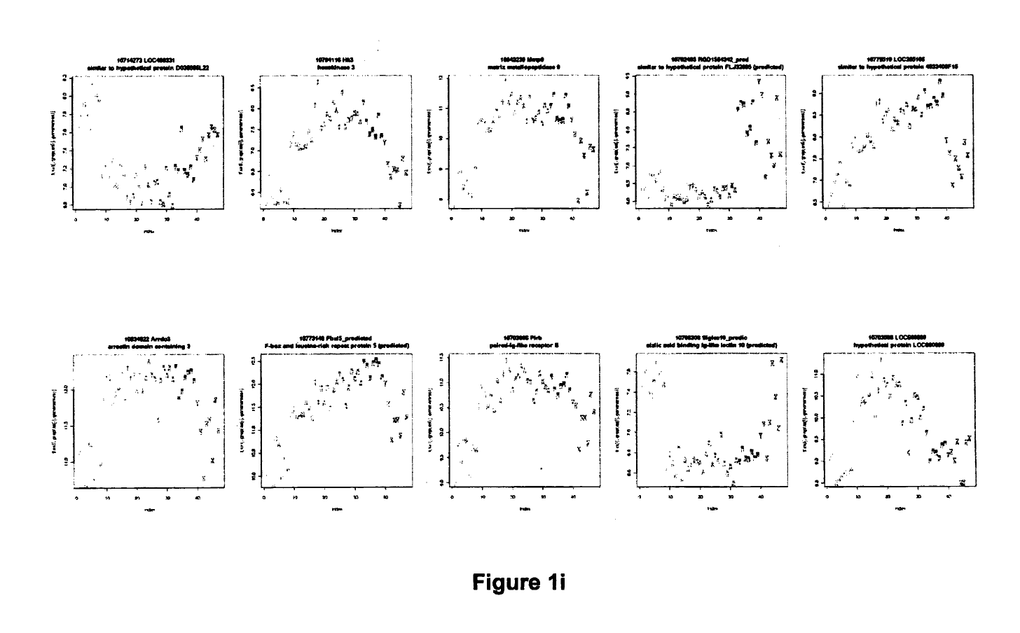

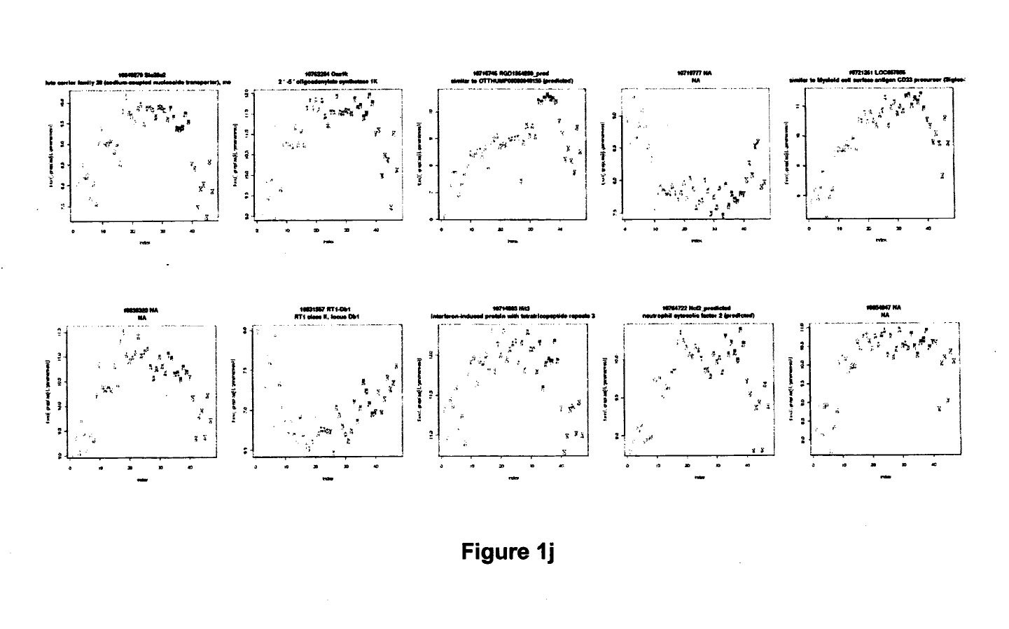

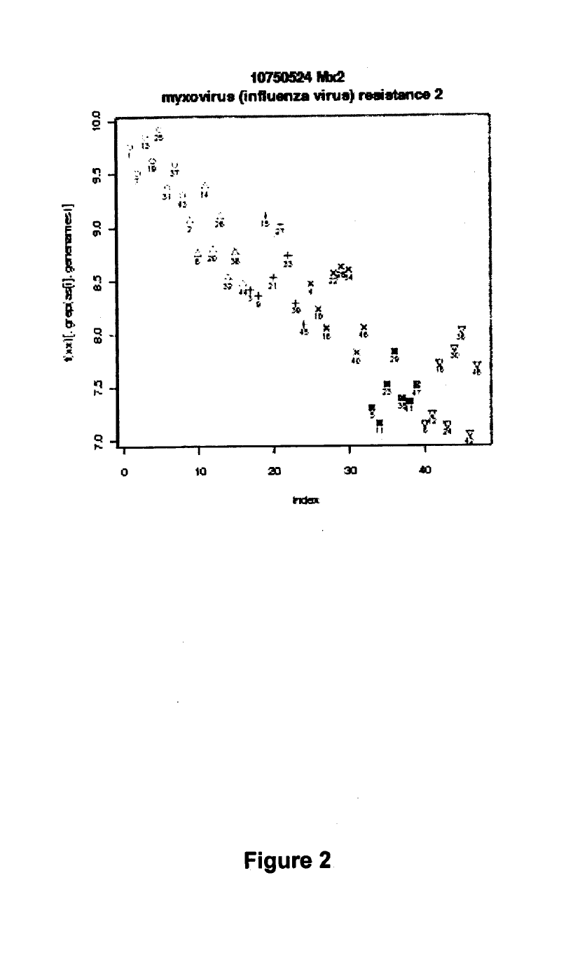

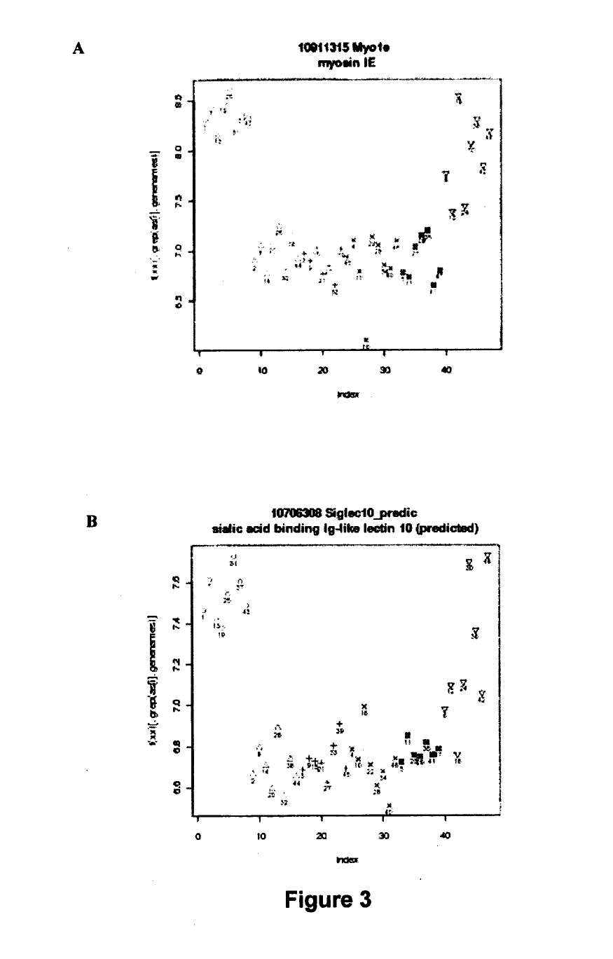

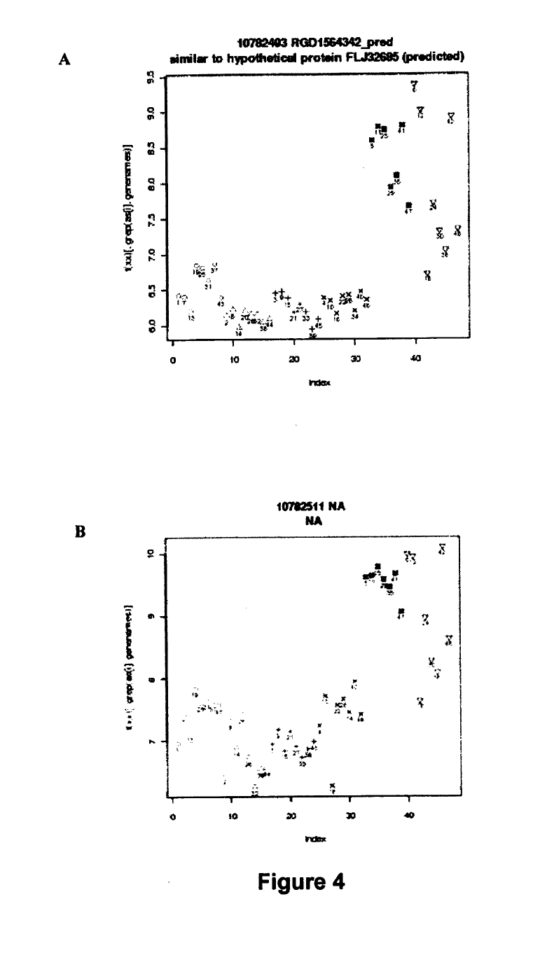

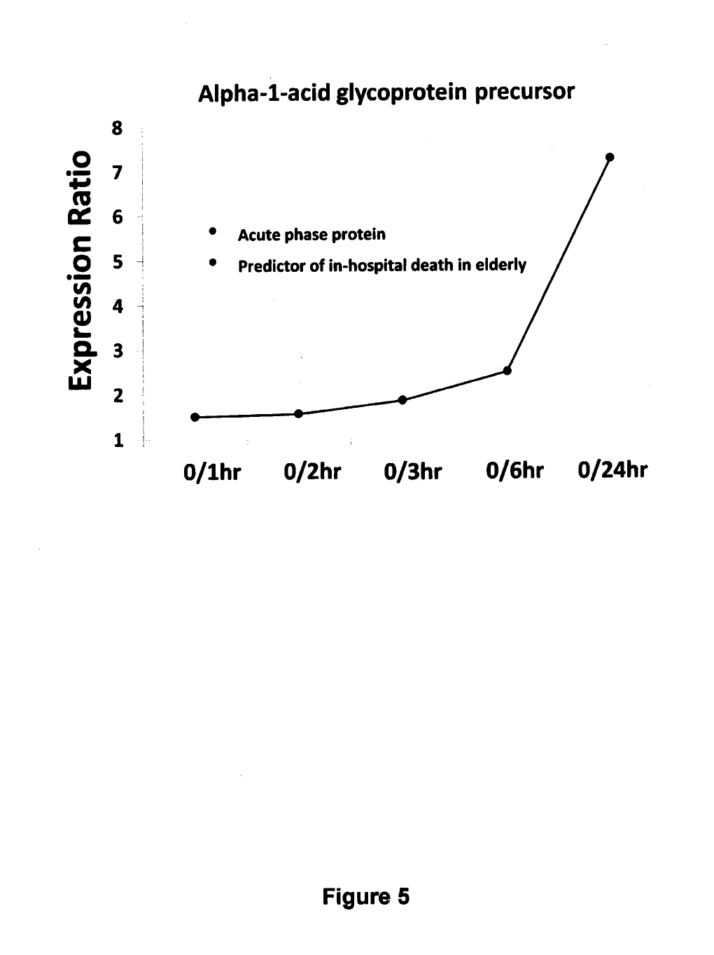

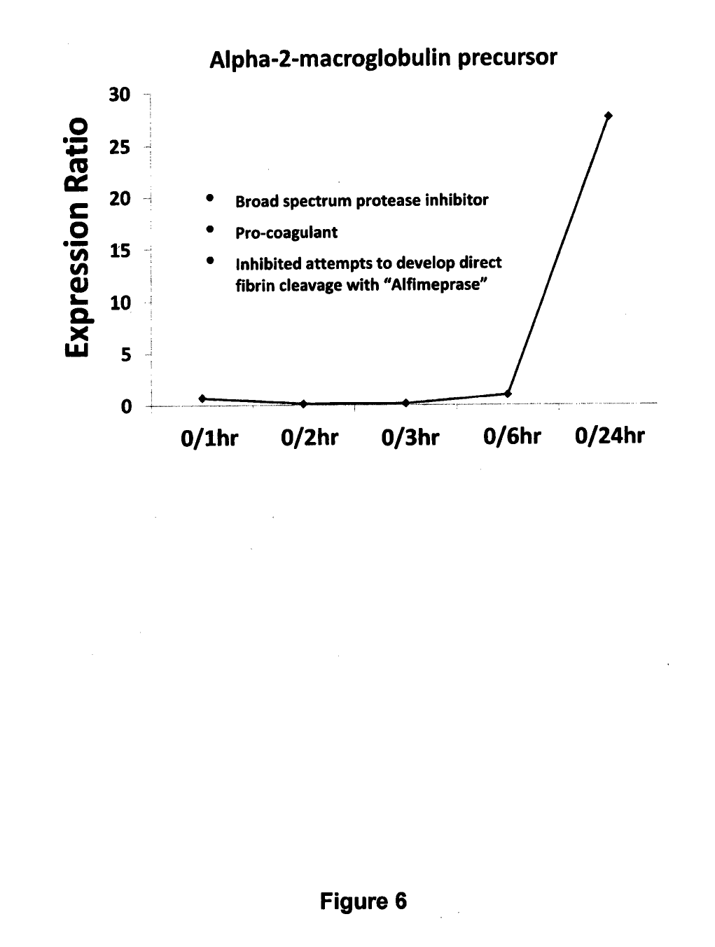

Expression studies of the biomarkers are provided graphically in FIGS. 1 through 6. The levels of biomarkers are conveniently subjected to multivariate statistical analysis. The biomarkers are referred to by Affymetrix gene identifier number. In summary, a list of biomarkers based on expression profile is provided below:

Steady decline with time of onset
10750524 (Mx2, myxovirus (influenza virus) resistance 2)
Steady increase to 6 hours from onset then falling
10940615
10926252

### CINC-1

Rapid early rise then rapid fall by 6 hours from onset
10722208 (Mrga10, nuclear receptor Mrga10 RF-amide G protein-coupled receptor)
10707142 (Ldhc, lactate dehydrogenase C)
10831940 (Fkbp5, FK506 binding protein 5)
10828832
Rapid early rise then persistent high expression to 6 hours from onset followed by slow fall by 24 hours
10922816 (Il1r2, Interleukin 1 receptor, type II)
10924245 (Il8rb, Interleukin 8 receptor, beta)
10718351 (Fpr1; formyl peptide receptor 1)
10854847
10703666 (Pirb, paired-Ig-like receptor B)
10721261 (LOC687856, similar to myeloid cell surface antigen CD33 precursor)
Steady rise to 6 hours, normalised by 24 hours from onset
10775519 (LOC305166, similar to hypothetical protein 4933408F15)
10849279 (Slc28a2, Solute carrier family 28 (sodium-coupled nucleoside transporter))
10774383 (Vps54, Vacuolar protein sorting 54 [yeast])
10754363
10751434
Rapid fall by 1 hour, persistent suppression to 6 hours, then normalising by 24 hours from onset
10763768 (Faim3, Fas apoptotic inhibitory molecule)
10748273 (Cd79b, CD79B antigen)
10769765
10911315 (Myo1e, myosin 1E)
10706308 (Siglec10 sialic acid binding Ig-like lectin 10)
No change of expression to 3 hours then increase by 6 hours from onset
10782493 (RGD1564342, similar to hypothetical protein FLJ32685)
10782511
Alpha-1-acid glycoprotein precursor
No change of expression to 6 hours then increase by 24 hours from onset
Alpha-2-macroglobulin precursor

Those skilled in the art will appreciate that aspects of aspects described herein are susceptible to variations and modifications other than those specifically described. It is to be understood that these aspects include all such variations and modifications. These aspects also include all of the steps, features, compositions and compounds referred to or indicated in this specification, individually or collectively, and any and all combinations of any two or more of the steps or features.

### BIBLIOGRAPHY

Donnan et al. (2008) Lancet 371:1612-1623
Hacke et al. (2004) Lancet 363:768-774
Hacke et al. (2008) N Engl J Med 359:1317-1329
Lees et al. (2010) Lancet 375:1695-1703
Smyth et al. (2005) LIMMA:Linear Models for microarray data In Bioinformatics and Computational Biology Solutions using R and Bioconductor R. Gentleman V Carey, S. Dudoit, R Irizarry W Huber (Eds), Springer, New York: 397-420
Strong et al. (2007) Lancet Neurol 6:182-187

1-29. (canceled)

30. A method for monitoring progression of a condition or event of a vascular system in a subject, said method comprising determining the levels or ratios of levels of 2 or more biomarkers selected from the list set forth in Table 1, wherein the levels or ratios of levels of the biomarkers alter during evolution of the condition or event, wherein the levels or ratios of levels of the biomarkers relative to a control correlate to the extent of progression of the condition or event.

31. The method of claim 30, wherein the vascular system is the cerebrovascular system.

32. The method of claim 30, wherein the condition or event is an ischemic condition or event.

33. The method of claim 30, wherein the condition or event is selected from ischemic stroke, ischemic myocardial infarction and vascular dementia.

34. The method of claim 31, wherein the condition or event is hemorrhagic stroke.

35. The method of claim 30, wherein the 2 or more biomarkers are selected from the biomarkers listed in Table 2.

36. The method of claim 33, wherein the correlation between onset of a stroke and progression is determined by measuring changes in expression levels of 2 or more of the following genes listed by Affymetrix ID number:
Steady decline with time of onset
10750524
Steady increase to 6 hours from onset then falling
10940615
10926252
Rapid early rise then rapid fall by 6 hours from onset
10722208
10707142
10831940
10828832
Rapid early rise then persistent high expression to 6 hours from onset followed by slow fall by 24 hours
10922816
10924245
10718351
10854847
10703666
10721261
Steady rise to 6 hours, normalised by 24 hours from onset
10775519
10849279
10774383
10754363
10751434
Rapid fall by 1 hour, persistent suppression to 6 hours, then normalising by 24 hours from onset
10763768
10748273
10769765
10911315
10706308
No change of expression to 3 hours then increase by 6 hours from onset
10782493
10782511.

37. The method of claim 30, wherein the subject is human.

38. The method of claim 37, wherein the method allows stratification of a human subject on the basis of whether or not the subject is within a therapeutic window to receive a particular treatment.

39. The method of claim 38, wherein the treatment is thrombolytic intervention.

40. The method of claim 39, wherein the therapeutic window to receive thrombolytic treatment is defined by levels or ratios of levels of the biomarkers being approximately the same as the levels at from 0 hours to 4.5 hours after onset of the condition or event.

41. A method of treating a subject having or suspected of having suffered an acute ischemic condition or event of a vascular system, said method comprising:
determining the levels or ratios of levels of 2 or more biomarkers selected from the list set forth in Table 1, wherein the levels or ratios of levels of the biomarkers alter during evolution of the condition or event, wherein the levels or ratios of levels of the biomarkers relative to a control correlate to the extent of progression of the condition or event and wherein the levels or ratios of levels of the biomarkers approximately equate to the levels or ratios of levels present from between 0 hours to 4.5 hours after onset of the condition or event; and,
administering to the subject a thrombolytic agent if the levels or ratios of levels of the biomarkers indicate that physiologically the subject is within the 0 to 4.5 hour time period from onset.

42. A method of treating a subject having or suspected of having suffered a condition or event of a vascular system, said method comprising:
determining the levels or ratios of levels of 2 or more biomarkers selected from the list set forth in Table 2, wherein the levels or ratios of levels of the biomarkers alter during evolution of the condition or event, wherein the levels or ratios of levels of the biomarkers relative to a control correlate to the extent of progression of the condition or event and wherein the levels or ratios of levels of the biomarkers approximately equate to the levels or ratios of levels present from between 0 hours to 4.5 hours after onset of the condition or event; and,
administering to the subject a thrombolytic agent if the levels or ratios of levels of the biomarkers indicate that physiologically the subject is within the 0 to 4.5 hour time period from onset.

43. The method of claim 41, wherein the vascular system is the cerebrovascular system.

44. The method of claim 41, wherein the condition or event is selected from ischemic stroke, ischemic myocardial infarction, and vascular dementia.

45. The method of claim 43, wherein the condition or event is a hemorrhagic stroke.

46. The method of claim 44, wherein the correlation between onset of a stroke and progression is determined by measuring changes in expression levels of 2 or more of the following genes listed by Affymetrix ID number:
Steady decline with time of onset
10750524
Steady increase to 6 hours from onset then falling
10940615
10926252
Rapid early rise then rapid fall by 6 hours from onset
10722208
10707142
10831940
10828832
Rapid early rise then persistent high expression to 6 hours from onset followed by slow fall by 24 hours
10922816
10924245
10718351
10854847
10703666
10721261
Steady rise to 6 hours, normalised by 24 hours from onset
10775519
10849279
10774383
10754363
10751434
Rapid fall by 1 hour, persistent suppression to 6 hours, then normalising by 24 hours from onset
10763768
10748273
10769765
10911315
10706308
No change of expression to 3 hours then increase by 6 hours from onset
10782493
10782511.

47. The method of claim 48, wherein from 2 to about 20 biomarkers are selected.

48. A diagnostic assay for determining an extent of progression of a condition or event of a vascular system in a subject comprising two or more biomarkers selected from Table 1.

49. The diagnostic assay of claim 48, wherein the vascular system is the cerebrovascular system.

50. The diagnostic assay of claim 48, wherein the condition or event is an ischemic condition or event.

51. The diagnostic assay of claim 48, wherein the condition or event is selected from an ischemic stroke, ischemic myocardial infarction, hemorrhagic stroke and vascular dementia.

52. The diagnostic assay of claim 48, wherein the two or more biomarkers are selected from the biomarkers listed in Table 2.

53. A method of allowing a user to determine the status of a subject with respect to an acute condition or event of a vascular system such as the cerebrovascular system, said method comprising the user receiving data, in the form of levels or ratios of levels of 2 or more biomarkers selected from Table 1 which correlate with the evolution of the condition or event; wherein the levels or ratios of levels of the biomarker relative to a control provide a correlation to the presence, state, classification or progression of the condition or event, from the user via a communications network which processes the data and which determines the status of the subject in accordance with a comparison with pre-determined values, wherein the results indicating the status of the subject are transferred to the user via the communications network.
